(19) United States
(12) Patent Application Publication
(10) Pub. No.: US 2004/0138272 A1
(43) Pub. Date: Jul. 15, 2004

(54) 1,4-Substituted cyclohexane derivatives

(76) Inventors: Lisa McKerracher, Verdun (CA); Eryk Thouin, Montreal (CA); William D. Lubell, Montreal (CA)

Correspondence Address: BROUILLETTE KOSIE PRINCE, 25th Floor, 1100 Rene-Levesque Boulevard West, Montreal, QC, H3B 5C9, CA

(21) Appl. No.: 10/651,283

(22) Filed: Aug. 29, 2003

(30) Foreign Application Priority Data
Sep. 3, 2002 (CA) 2,400,996

(51) Int. Cl. A61K 31/44; A61K 31/40; A61K 31/165; C07D 207/46

(52) U.S. Cl. 514/357; 514/408; 514/620; 514/534; 546/335; 546/336; 548/571; 560/125; 564/191

(57) ABSTRACT

Allylic compounds represented by the formula (I) are provided,

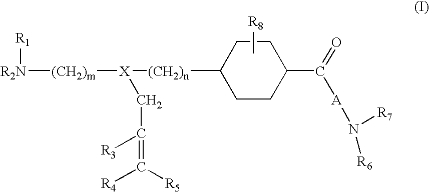

wherein each of R₁ to R₈, m, n, A and X are as defined in the Specification. These compounds can inhibit Rho kinase, and can find utility in repair of damaged nerves in the central and peripheral nervous system by inducing axon growth and regeneration, and in the treatment by inhibition of Rho kinase in disease states in which Rho kinase is implicated. The compounds are relatively cell permeable and pharmaceutical compositions thereof can promote neurite growth and are also useful for the prevention of cell proliferation in malignant deseases.

### FIELD OF THE INVENTION

The present invention relates to molecules or compounds which are inhibitors of Rho kinase, and in particular to compounds that are membrane permeable and that can promote neurite growth, and to pharmaceutical compositions comprising these compounds. The present invention also relates to the use of the compositions and compounds to repair damage to nerve cells and components of nerve structures in the nervous system, to prevent ischemic cell death, and to treat various disease states wherein the treatment comprises inactivation of Rho kinase.

### BACKGROUND

Traumatic injury of the spinal cord results in permanent functional impairment. Most of the deficits associated with spinal cord injury result from cell death and the loss of axons in the spinal neuronal population that are damaged in the central nervous system (CNS) which is comprised of nerves in the spinal cord and brain. Neurodegenerative diseases of the CNS are also associated with cell death and axonal loss. Representative diseases of the CNS include stroke, human immunodeficiency virus (HIV) dementia, prion diseases, Parkinson's disease, Alzheimer's disease, multiple sclerosis, traumatic brain injury, and glaucoma. The ability to stimulate growth of axons from the affected or diseased neuronal population would improve recovery of lost neurological functions, and protection from cell death can limit the extent of damage in the CNS. For example, following a white matter stroke, axons are damaged and lost, even though the neuronal cell bodies are alive, and stroke in grey matter kills many neurons and non-neuronal (glial) cells. Neuroprotective agents can potentially limit damage after stroke. Compounds which promote growth and are neuroprotection agents are especially good candidates for treatment of stroke and neurodegenerative diseases.

Although the following discussion will generally relate to use of Rho kinase inhibitors to treat a traumatically damaged nervous system, the compositions and methods of this invention may also be applied to treatment of diseases and cell damage arising from disease states and causes, such as during stroke, multiple sclerosis, HIV dementia, Parkinson's disease, Alzheimer's disease, ALS, traumatic brain injury, prion diseases or other diseases of the CNS where axons are damaged in the CNS environment, and includes those disease states identified herein.

Rho kinase is a target for treatment of cancer and metastasis (Clark et al (2000) Nature 406:532-535), and hypertension (Uehata et al. (1997) Nature 389:990), and RhoA is reported to have a cardioprotective role (Lee et al. FASEB J. 15:1886-1884). Rho kinase inhibitors have been used in animal models to treat eye diseases such as glaucoma (Honjo et al., 2001; Rao et al., 2001), and cancer cell migration and metastasis (Imamura et al., 2000; Sahai et al., 1999; Takamura et al.,2001). The effect of the Rho signalling pathway on smooth muscle relaxation has led to the identification of Rho signalling antagonists as effective in treatment of hypertension (Chitaley et al., 2001 Curr Hypertens Rep. 3:139-144.; Uehata et al., 1997 Nature 389:990), asthma (Iizuka et al., 2000 Eur J Pharmacol. 406:273-9.; Nakahara et al., 2000 Eur J Pharmacol. 389:103-6.), and vascular disease including thrombosis (Iizuka et al., 2000 Eur J Pharmacol. 406:273-9; Miyata et al., 2000 Arterioscler Thromb Vasc Biol. 20:2351-8.; Nakahara et al. . Eur J Pharmacol. 389:103-6, 2000; Robertson et al., 2000. Br J Pharmacol. 131:5-9).

A membrane permeable, non-toxic inhibitor of Rho kinase of this invention can have many potential medical applications. The compounds of the present invention, which are Rho kinase inhibitors, are expected to be useful in the therapeutic treatment of a variety of diseases where inhibition of Rho kinase activity is required. The compounds of the present invention can affect smooth muscle and endothelial cells and can find useful application in a variety of therapeutic aspects such use on stents, as coated stents to prevent restenosis.

Traumatic injury of the spinal cord results in permanent functional impairment. Axon regeneration does not occur in the adult mammalian CNS because substrate-bound growth inhibitory proteins block axon growth. While compounds such as trophic factors can enhance neuronal differentiation and stimulate axon growth in tissue culture, most factors that enhance growth and differentiation are not able to promote axon regenerative growth on inhibitory substrates. To demonstrate that a compound known to stimulate axon growth in tissue culture most accurately reflects the potential for therapeutic use in axon regeneration in the CNS, it is preferable for the cell culture studies to include the demonstration that a compound can permit axon growth on growth inhibitory substrates. Trophic and differentiation factors that stimulate growth on permissive substrates in tissue culture include neurotrophins such as nerve growth factor (NGF) and brain-derived growth factor. NGF, however, does not promote growth on inhibitory substrates (Lehmann, et al. 1999. J. Neurosci. 19: 7537-7547) and it has not been effective in promoting axon regeneration in vivo. Brain derived neurotrophic factor (BDNF) is not effective to promote regeneration in vivo either (Mansour-Robaey, et al. J. Neurosci. (1994) 91: 1632-1636). BDNF does not promote neurite growth on growth inhibitory substrates (Lehmann et al supra).

Cell death can occur by two major mechanisms, necrosis and apoptosis. While necrotic cell death results in cell lysis, cellular apoptosis is programmed cell death that results in the tidy packaging of cells that die to prevent the release of cellular contents. Apoptosis is characterized morphologically by cell shrinkage, nuclear pyknosis, chromatin condensation, and blebbing of the plasma membrane. Traumatic injury and ischemia can lead to apoptosis of both neurons and non-neuronal cells, and this cell death is responsible for functional deficits after injury or ischemia. A cascade of molecular and biochemical events is associated with apoptosis including activation of an endogenous endonuclease that cleaves DNA into oligonucleosomes detectable as a ladder of DNA fragments in agarose gels. Apoptotic endonucleases not only affect cellular DNA by producing the classical DNA ladder but also generate free 3′-OH groups at the ends of these DNA fragments. A technique called Tunel labeling labels DNA fragments as a means to detect apoptotic cells.

The Rho kinase regulates axon growth and regeneration, cell motility and metastasis, smooth muscle contraction, and apoptosis, and is an important target for therapeutic treatment in many disease applications, including repair in the central nervous system. It is an advantage that the compounds and compositions of the present invention inhibit the activity of Rho kinase. These compounds and compositions can be advantageous over C3 and C3-like fusion proteins because, since they are not peptides or proteins, they will not generate an unwanted immune response. It is a further advantage that the compounds of this invention are cell permeable. It is another advantage of this invention that the novel compounds and compositions disclosed herein can promote repair of nerve cells and of nerve structure when applied to an injured mammalian central nervous system. Compounds and compositions of this invention can promote neurite growth on growth inhibitory substrates.

Although the novel compounds and compositions of the present invention can be useful to facilitate regeneration of axons and in neuroprotection, it is to be understood that the compounds and compositions may be exploited in other contexts as shall be mentioned herein, including with respect to treatment of diseases such as cancers.

Rho kinase inhibitors of this invention can have potential therapeutic use in the treatment of cancer and of malignant transformations and abnormal proliferation of cells. Rho kinase is activated by Rho and Rho kinase inhibitors block Rho signaling. The number of Rho family regulatory proteins in which mutations have been found in clinical oncology samples provides justification for perturbation of Rho signaling as a therapeutic modality. Those with specificity for Rho include the DLC1 gene in hepatocellular carcinoma, p-190-A, which is in a region that is altered in gliomas and astrocytomas, GRAF, which has loss of function mutations in leukemia, and LARG, which found in some a gene fusions found in acute myeloid leukema (Jaffe and Hall, 2002 Adv. Cancer Res. 57-80). Genetically engineered point mutations activate RhoA and induce cellular transformation in vitro (reviewed by Khosravi-Far et al.,1998. Adv. Cancer Res. 65: 57-107). Many experiments in the scientific literature with the Rho kinase inhibitor Y-27632 demonstrate that inhibiting Rho kinase is effective in preventing metastasis.

A Rho kinase inhibitor, trans-4-amino(alkyl)-1-pyridylcarbamoylcyclohexane compound, designated as Y-27632, is available from Calbiochem. This compound is described in U.S. Pat. No. 4,997,834, the entire content of which is incorporated herein by reference. U.S. Pat. No. 6,218,410, the entire content of which is incorporated herein by reference, discloses a method for inhibiting Rho kinase.

Other types of compounds are described in U.S. Pat. No. 5,478,838, the entire content of which is incorporated herein by reference.

Y-27632 can relax smooth muscle and increase vascular blood flow. Y-27632 is a small molecule that can enter cells and is not toxic in rats after oral administration of 30 mg/kg for 10 days. Effective doses for the use of this compound are approximately 30 μM. It reduces blood pressure in hypertensive rats, but does not affect blood pressure in normal rats. This has led to the identification of Rho signalling antagonists in treatment of hypertension (Somlyo, 1997 Nature 389:908; Uehata et al., 1997 Nature 389:990; Chitaley et al., 2001a Curr. Hypertension Rep. 3:139).

A partial list of where the Rho kinase inhibitor Y-27632 has been tested for the disease applications is as follows:

Hypertension (Uehata et al., 1997 IBID; Chitaley et al., 2001a IBID; Chrissobolis and Sobey, 2001 C. Circ. Res 88:774);

Asthma (Iizuka et al., 2000 Eur. J. Pharmacol 406:273; Nakahara et al. Eur. J. Pharmacol 389:103, 2000);

Pulmonary vasoconstriction (Takamura et al., 2001 Hepatology 33:577);

Vascular disease (Miyata et al., 2000 Thromb Vasc Biol 20:2351; Robertson et al., 2000 Br. J. Pharmacol 131:5);

Penile erectile dysfunction (Chitaley et al., 2001b Nature Medicine 7:119; Mills et al., 2001 J. Appl. Physiol. 91:1269; Rees et al., Br. J. Pharmacol 133:455 2001);

Glaucoma (Honjo et al., 2001 Methods Enzymol 42:137; Rao et al., 2001 Invest. Opthalmol. Urs. Sci. 42:1029);

Cell transformation (Sahai et al., 1999 Curr. Biol. 9:136-5);

Prostate cancer metastasis (Somlyo et al., 2000 BBRC 269:652);

Hepatocellular carcinoma and metastasis (Imamura et al., 2000; Takamura et al., 2001);

Liver fibrosis (Tada et al., 2001 J. Hepatol 34:529; Wang et al., 2001 Am. J. Respir. Cell Mol Biol. 25:628);

Kidney fibrosis (Ohki et al., J. Heart Lung Transplant 20:956 2001);

Cardioprotection and allograft survival (Ohki et al., 2001 IBID); and

Cerebral vasospasm (Sato et al., 2000 Circ. Res 87:195).

The compounds or inhibitors in accordance with the present invention provide an alternative with respect to known Rho kinase inhibitors such as Rho kinase inhibitory Y-27632. A compound or inhibitor in accordance with the present invention, when compared with Y-27632, can exhibit different and improved kinase inhibition profiles and/or also promote better axon regeneration when tested in vivo.

The present invention relates to an alternate group of compounds for advantageously inhibiting the activity of Rho kinase. These compounds may be advantageous be exploited over C3 and C3-like fusion proteins because, since they are not peptides or proteins, they will not generate an unwanted immune response; and are relatively readily cell permeable. The present in a further aspect relates to compounds for promoting repair when applied to the injured mammalian central nervous system, i.e. promote neurite growth. The present also relates to compounds for providing an alternative route for the prevention of cell proliferation in malignant deseases.

Other known Rho kinase inhibitor compounds include the following:

a) The compound NHM-1152 has been reported to be a Rho kinase inhibitor and acts as a vascular relaxant (Tanaka, 1998 Naunyn-Schmiedeberg's Archives of Pharmacology 358 (suppl.)). It is under preclinical development for vascular vasospasm in Japan.

b) Hydroxy fasudil has been tested for use in stroke after intravenous application and was found to reduce infarct volume and improve outcomes (Satoh et al. Life-Sci. 69:1441, 2001). It has also been tested for anti-ischemic properties in vasospastic angina (Sato et al., 2001 Jpn. J. Pharmacel 87:34), and inhibits neutrophil migration in ischemic brain.

c) A fasudil compound called HA-1077 (apparently the same as U-46610), being developed in Japan, is an antivasospasm drug that inhibits Rho kinase. In addtion to its vasodilatory action, fasudil improves cerebral hemodynamic activity and inhibits production of superoxide anion by neurotrophils (Hara et al., 2000 J. Neurosurg 93:94; Hitomi et al., 2000 Life Sci. 67:1929; Toshima et al. Stroke 31:2245, 2000). It is effective in spinal cord injury (Hara et al., IBID 2000) and stroke (Toshima et al., 2000 IBID). In Japan, fasudil is used clinically to treat patients who have suffered a subarachnoid hemorrhage, and clinical trials for cerebral infarction have begun (Hara et al., 2000 IBID)

The present invention, in accordance with one aspect, in particular (but not limited thereto) pertains to the field of mammalian nervous system repair (e.g. repair of a central nervous system (CNS) lesion site or a peripheral nervous system (PNS) lesion site), axon regeneration and axon sprouting, neurite growth and protection from neurodegeneration and ischemic damage. The compounds and compositions of the present invention can find use in repair in a mammal of a component of a nervous system such as a central nervous system (CNS) lesion site or a peripheral nervous system (PNS) lesion site, in axon regeneration and/or axon sprouting, in neurite growth and/or protection from neurodegeneration and ischemic damage. Targeting intracellular signalling mechanisms involving Rho and the Rho kinase for promoting axon regeneration has been proposed (see, for example, Canadian Patent application 2,304,981 (McKerracher et al)). The Rho family GTPases regulates axon growth and regeneration (Lehmann, et al. 1999. J. Neurosci. 19: 7537-7547). Inactivation of Rho with Clostridium botulinum C3 exotransferase (hereinafter simply referred to as C3) can stimulate regeneration and sprouting of injured axons ; Activated Rho stimulates its downstream effector Rho kinase, and inactivation of Rho kinase can promote axon growth (Bito,H. et al., 2000. Neuron 26: 431-441). More importantly, Rho kinase inhibitor can promote axon growth on growth inhibitory substrates and can promote repair in the injured CNS.

It has been proposed to use various Rho kinase inhibitors to stimulate or promote regeneration of (cut) axons, i.e. nerve lesions; see, for example, Canadian Patent application nos. 2,304,981 (McKerracher et al) and 2,325,842 (McKerracher); Derghan et al. (2002) J. Neurosci. 22:6570. These patent application documents propose the use of known Rho antagonists such as for example C3, chimeric C3 proteins, etc. (see below) as well as substances selected from among known trans-4-amino(alkyl)-1-pyridyl-carbamoylcyclohexane compounds (see above) or Rho kinase inhibitors for use in the regeneration of axons. C3 inactivates Rho by ADP-ribosylation and is fairly non-toxic to cells (Dillon and Feig (1995) Methods in Enzymology: Small GTPases and their regulators Part. B.256:174-184).

While the compositions and methods of this invention will be generally described in terms of or be directed at repair in the CNS, the inventive compositions and techniques described herein may be extended to use in many other diseases including, but not restricted to, cancer, metastasis, hypertentension, cardiac disease, stroke, diabetic neuropathy, and neurodegenerative disorders such as stroke, Alzheimer's disease, Parkinson's disease, amyotrophic lateral sclerosis (ALS). Treatment with a compound of the present invention including a pharmaceutically acceptable salt thereof, (e.g. Rho kinase inhibitors) may be used to enhance the rate of axon growth of nerves such as peripheral nerves and thereby be effective for repair of damaged peripheral nerves after surgery, for example after reattaching severed limbs or after prostate surgery. Also, treatment with a compound of the present invention including a suitable salt thereof, can be effective for the treatment of various peripheral neuropathies (such as diabetic neuropathy) because of its axon growth promoting effects.

### STATEMENT OF INVENTION

The present invention in an aspect provides a compound of formula (I),

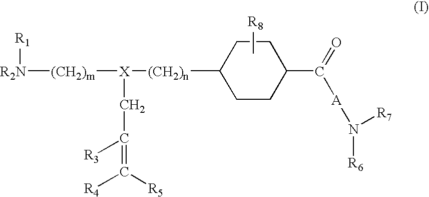

wherein X is CH or N

m is 0, 1, 2 or 3

and

n is 0, 1, 2 or 3

wherein

R₁ is selected from the group consisting of H, alkyl, cycloalkyl, cycloalkylalkyl, aryl (e.g.phenyl), aralkyl (e.g. benzyl), and a heteroaryl, a ring group optionally having a substituent on the ring thereof, and

R₂ is selected from the group consisting of H, alkyl, cycloalkyl, cycloalkylalkyl, aryl (e.g.phenyl), aralkyl (e.g. benzyl), and a heteroaryl, a ring group optionally having a substituent on the ring thereof, or

R₁ and R₂ together with the adjacent nitrogen atom form a heterocyclic group (single or fused ring structure, e.g. an aromatic heterocyclic group) optionally having in the ring an oxygen atom, a sulfur atom or an additional nitrogen atom, the heterocyclic group optionally having a substituent on the ring thereof (e.g. an optionally substituted nitrogen ring atom),

wherein

R₃ is selected from the group consisting of H, halo (e.g. Cl, F, I, Br), alkyl, cycloalkyl, cycloalkylalkyl, aryl (e.g.phenyl), aralkyl (e.g. benzyl), heteroaryl (e.g. a heteroaryl as defined hereinbelow, including or for example a heteroaryl as described with respect to R₁ and R₂ together, R₇, etc.), a ring group optionally having a substituent on the ring thereof,

R₄ is selected from the group consisting of H, halo (e.g. Cl, F, I, Br), alkyl, cycloalkyl, cycloalkylalkyl, aryl (e.g.phenyl), aralkyl (e.g. benzyl), a heteroaryl (e.g. a heteroaryl as defined hereinbelow, including or for example a heteroaryl with respect to R₁ and R₂, R₇, etc.), a ring group optionally having a substituent on the ring thereof,

R₅ is selected from the group consisting of H, halo (e.g. Cl, F, I, Br), alkyl, cycloalkyl, cycloalkylalkyl, aryl (e.g.phenyl), aralkyl (e.g. benzyl), heteroaryl (e.g. a heteroaryl group such as defined with respect to R₁ and R₂, R₇, etc.), a ring group optionally having a substituent on the ring thereof,

R₆ is selected from the group consisting of H, alkyl, aryl (e.g. phenyl), heteroaryl, heteroarylalkyl and aralkyl (e.g. benzyl),

R₇ is selected from the group consisting of aryl groups (e.g.phenyl), aralkyl groups (e.g. benzyl), and heterocyclic groups (single or fused ring structures e.g. aromatic heterocyclic groups—heteroaryl or heteroarylalkyl groups) containing at least one nitrogen atom in the ring structure thereof, a ring group optionally having a substituent on the ring thereof (e.g. a ring group may be an optionally substituted nitrogen ring atom; a substituted ring group may be an amino or diamino aryl group, an amino or diamino aralkyl group (e.g. 3-(diaminomethyl)-benzyl), etc.),

R₈ is selected from the group consisting of H, alkyl, halo (e.g. fluoro, chloro, etc.) cycloalkyl, cycloalkylalkyl, aryl (e.g.phenyl), arylalkyl (e.g. benzyl), a ring group optionally having a substituent on the ring thereof, and

A is a single bond or is an unsubstituted straight chain alkylene group (e.g. methylene, ethylene, trimethylene, tetramethylene, etc.) or a straight chain alkylene group (e.g. methylene, ethylene (i.e. —CH₂CH₂—), trimethylene, tetramethylene, etc.) substituted by alkyl of 1 to 4 carbon atoms (e.g. methyl, ethyl, propyl).

In accordance with an aspect of the present invention when X is N, for the various general compound structures given herein, the general alkylene (e.g. allyl) group associated therewith may be replaced by the group Ra as defined hereinbelow (e.g. as with respect to Formula (II) below), the group Ra including the general alkylene (e.g. allyl) group.

The present invention in a particular aspect relates to compounds of formula (I) wherein X is CH (and A is a single bond). Thus the present invention relates to a compound of formula (Ia)

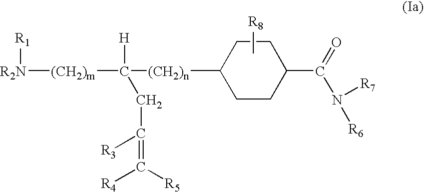

and pharmaceutically acceptable salts thereof

wherein m, n, R₁, R₂, R₃, R₄, R₅, R₆, R₇, and R₈ are as defined herein (i.e. as defined hereinabove as well as hereinbelow).

In accordance with the present invention R₂, R₃, R₄, R₅, R₆, and R₈ may for example each be H.

In accordance with the present invention R₁ may for example be selected from the group consisting of H, C₁ to C₆₋₁₀ alkyl, and benzyl.

The present invention in a further particular aspect relates to compounds of formula (I) wherein X is CH, m and n are each 0 (zero), R₂, R₃, R₄, R₅, R₆ and R₈ are each H and A is a single bond. Thus the present invention in an additional particular aspect relates to a compound of formula (Ib)

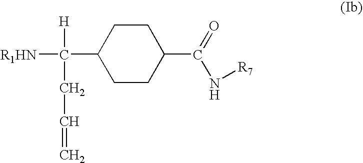

and pharmaceutically acceptable salts thereof

wherein R₁, and R₇ are as defined herein (i.e. as defined hereinabove as well as hereinbelow); e.g. R₁ may for example be selected from the group consisting of H, C₁ to C₆₋₁₀ alkyl, and benzyl.

The present invention in accordance with another aspect relates to a compound of formula (II)

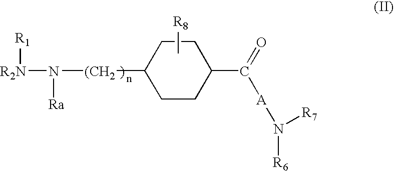

and pharmaceutically acceptable salts thereof

wherein A, R₁, R₂, R₆, R₇, and R₈ are as defined herein (i.e. as defined hereinabove as well as hereinbelow)

and

wherein

Ra is selected from the group consisting of H, alkyl, cycloalkyl, cycloalkylalkyl, aryl (e.g. phenyl), aralkyl (e.g. benzyl), arylalkylene, aryloxyaryl, heteroaryl, a ring group optionally having a substituent on the ring thereof and an alkylene group (e.g. allyl) of formula

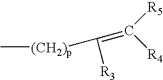

wherein p is 0, 1, 2 or 3, and R₃, R₄, and R₅, are as defined herein (i.e. as defined hereinabove as well as hereinbelow).

The present invention in accordance with a particular aspect relates to a compound of formula (IIa)

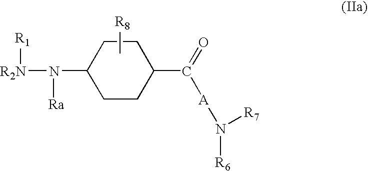

and pharmaceutically acceptable salts thereof

wherein A, Ra, R₁, R₂, R₆, R₇, and R₈ are as defined herein (i.e. as defined hereinabove as well as hereinbelow)

The present invention in a particular aspect relates to compounds of formula (II) wherein A is a single bond. Thus the present invention relates to a compound of formula (IIb)

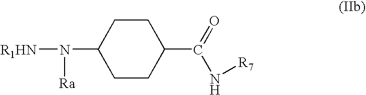

wherein Ra, R₁, and R₇ are as defined herein (i.e. as defined hereinabove as well as hereinbelow); e.g. R₁ may for example be selected from the group consisting of H, C₁ to C₆₋₁₀ alkyl, and benzyl.

In accordance with the present invention Ra may for example be selected from the group consisting of H, C₁ to C₈₋₁₀ alkyl, cyclohexyl(C₁ to C₃)alkyl (e.g. cyclohexylmethyl), phenyl(C₁ to C₃)alkyl (e.g. benzyl, 2′ (phenyl)ethyl, etc.), diphenyl(C₁ to C₃)alkyl (e.g. 2′, 2′ (diphenyl)ethyl, etc.), phenyl(C₂ to C₃)alkylene (e.g. 2′ (phenyl)ethylenyl, 3′ (phenyl)prop-2′-enyl, etc.), benzyloxybenzyl (e.g. 4′-(benzyloxy)benzyl, etc. ) and allyl.

The present invention in another aspect provides a compound of formula (III)

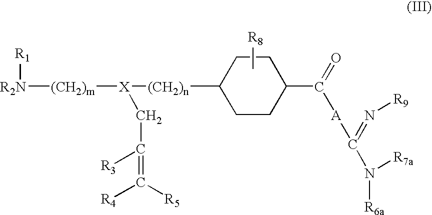

wherein X, A, m, n, R₁, R₂, R₃, R₄, R₅, and R₈ are as defined herein (i.e. as defined hereinabove as well as hereinbelow),

and

wherein

R₆ₐ is selected from the group consisting of H, alkyl, heteroaryl, heteroarylalkyl, aryl (e.g. phenyl) and aralkyl (e.g. benzyl),

R₇ₐ is selected from the group consisting of H, heteroaryl, heteroarylalkyl, aryl groups (e.g.phenyl), aralkyl groups (e.g. benzyl),

and

R₉ is selected from the group consisting of H, alkyl, aryl (e.g. phenyl) and aralkyl (e.g. benzyl).

As may be appreciated from the above formulae, the present invention relates to compounds which, for example, comprise an allylic group or a hydrazine proximal to a cyclohexane ring.

It is to be understood herein that if a formula for a fused ring structure is provided with a floating bond (e.g. single bond) for connecting the structure to another component or element or with a floating substituent group, the floating bond and/or substituent group (unless otherwise dictated by the structure) may be attached to either (or any) of the ring moieties.

In accordance with the present invention a heterocyclic group may optionally have a substituent on the ring thereof, (e.g. alkyl, halo, etc.).

In accordance with the present invention R₇ may for example be selected from the group consisting of

a group of formula (i)

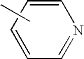

a group of formula (ii)

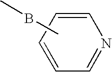

a group of formula (iii)

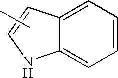

a group of formula (iv)

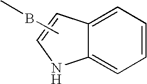

a group of formula (v)

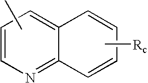

a group of formula (vi)

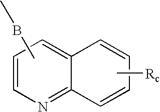

a group of formula (vii)

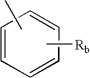

a group of formula (viii)

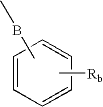

a group of formula (ix)

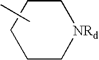

a group of formula (x)

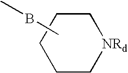

a group of formula (xi)

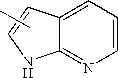

a group of formula (xii)

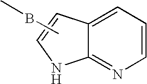

a group of formula (xiii)

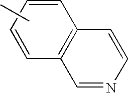

a group of formula (xiv)

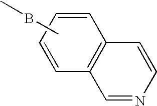

a group of formula (xv)

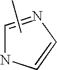

a group of formula (xvi)

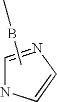

a group of formula (xvii)

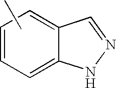

a group of formula (xviii)

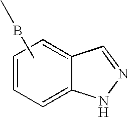

a group of formula (xix)

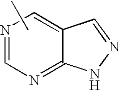

and

a group of formula (xx)

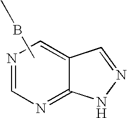

wherein B is alkylene (an unsubstituted straight chain alkylene group (e.g. methylene, ethylene, trimethylene, tetramethylene, etc.) or a straight chain alkylene group (e.g. methylene, ethylene, trimethylene, tetramethylene, etc.) substituted by alkyl of 1 to 4 carbon atoms), and R_{b} is selected from the group consisting of H, alkyl, amino, alkylamino, dialkylamino, R_{c} is selected from the group consisting of H, alkyl and R_{d} is selected from the group consisting of H, alkyl, aralkyl.

More particularly, in accordance with the present invention

the group of formula (i) may be

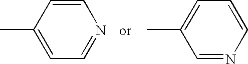

the group of formula (ii) may be

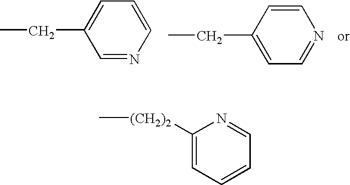

the group of formula (iii) may be

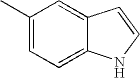

the group of formula (iv) may be

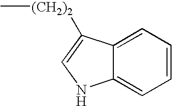

a group of formula (v) may be

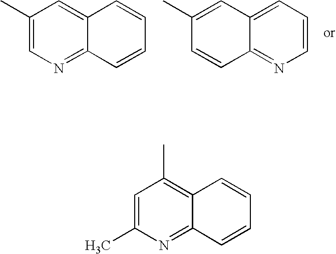

the group of formula (viii) may be

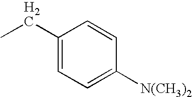

the group of formula (ix) may be

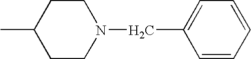

the group of formula (xi) may be

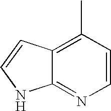

the group of formula (xiii) may be

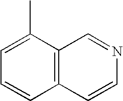

the group of formula (xv) may be

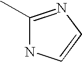

the group of formula (xvii) may be

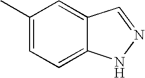

and

the group of formula (xix) may be

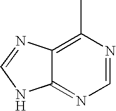

The present invention encompasses any and all of the various isomers of the compounds of formula (I), (Ib), (II), (IIa), (III) etc.; e.g. cis- or trans-geometrical isomers, R- and S-isomers, enantiomers, etc.. of the compounds of formula (I), (II), etc. and mixtures thereof; including, without limitation, as well as optical isomers and their racemates (i.e. compounds having an asymmetric carbon).

In accordance with the present invention an alkyl group or moiety (e.g. the alkyl moiety of an arylalkyl group) may be straight or branched and may comprise from 1 to 10 carbon atoms (e.g. alkyl of 1 to 6 carbon atoms, heptyl, octyl, nonyl or decyl, etc.); a cycloalkyl group or a. cycloalkyl moiety (e.g. the cycloalkyl moiety of a cycloalkylalkyl group) may comprise from 3 to 7 carbon atoms; an aryl group may be a single or fused ring structure—the ring structures may for example comprise up to 14 ring atoms (e.g. 14 ring carbon atoms); an alkylene group may comprise up to 5 carbon atoms. Thus an arylalkyl group such as for example phenylalkyl may include for example benzyl, phenylethyl, phenylpropyl, phenylbutyl and the like.

In accordance with the present invention a heterocyclic group may be a single or fused ring structure, (e.g. an aromatic heterocyclic group, i.e. a heteroaryl group which may be a single or fused ring structure—the ring structures may for example comprise up to 14 ring atoms (e.g. up to 14 ring atoms comprising at least one nitrogen ring atom)) optionally having in the ring an oxygen atom, a sulfur atom or an additional nitrogen atom, the heterocyclic group optionally having a substituent on the ring thereof (e.g. an optionally substituted nitrogen ring atom). A heteroaryl group may, for example have the basic ring forms as discussed herein with respect to R₇.

In accordance with the present invention a substituent may be an alkyl group, a halo group (e.g. Cl, Br, F, I), a carboxyl group, a carboxylalkyl group, etc.. If the substituent is with respect to an aryl or heteroaryl group or moiety, the substituent may be any substituent which alters the aromatic character of the aryl or heteroaryl group or moiety as desired or appropriate, for example by donating electron density to or by withdrawing electron density from the aryl or heteroaryl group or moiety.

The term “Rho antagonists” as used herein includes, but is not restricted to, (known) C3, including C3 chimeric proteins, and like Rho antagonists.

The term Rho kinase inhibitor relates to a compound that inactivates or reduces the ability of Rho kinase to phosphorylate downstream substrates.

The term “nerve injury site” refers to a site of traumatic nerve injury or of traumatic nerve damage, or of nerve injury or nerve damage or nerve abnormality caused by disease, particularly in a mammal. In one aspect a nerve injury can comprise a completely severed nerve, wherein a normally occurring nerve is severed or broken into at least two residual nerve parts comprising segments of the original nerve. In another aspect a nerve injury can comprise a partially severed nerve, wherein a normally occurring nerve is from about 1% to about 99% severed or broken at the site of injury to the original nerve, and wherein the original nerve remains from about 1% to about 99% in tact at the site of damage to the nerve. A nerve injury site may occur in a single nerve (e.g., in a sciatic nerve) or in a nerve tract or in a nerve structure comprised of many nerves (e.g., a nerve injury site can comprise a damaged region of the spinal cord). A nerve injury site may be in the central nervous system (e.g., in the brain and/or spinal cord) or in a peripheral nervous system or in any region of nerve in need of repair. A nerve injury site may form as a result of damage caused by stroke. A nerve injury site may be located in the brain and comprise damage to brain tissue which may occur, for example, as a result of a surgical procedure wherein a portion of normally connected brain tissue is cut or severed completely or partially into at least two parts or domains, or as a result of surgical removal of a brain tumour or as a result of therapy such as radiation therapy or chemotherapy such as can occur in the presence of or following removal of a cancerous lesion. A nerve injury site may result from stroke, Parkinson's disease, Alzheimer's disease, amyotrophic lateral sclerosis (ALS), diabetes or any other type of neurodegenerative disease.

It is further to be understood herein, that if a “group of substances”, “group of substituents”, “range” of a particular characteristic (e.g., temperature, concentration, time and the like) or the like is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,

with respect to the number of carbon atoms, the mention of the range of 1 to 10 carbon atoms is to be understood herein as incorporating each and every individual number of carbon atoms as well as sub-ranges such as, for example, 1 carbon atoms, 3 carbon atoms, 4 to 6 carbon atoms, etc.;

with respect to spatial geometry, compounds of formual (I), (Ib), (II), (IIa), etc. are to be understood as encompassing each and every individual isomer of the compounds of formual (I), (Ia), (II), (IIa), etc. and mixtures thereof e.g. cis- or trans-geometrical isomers of the Compounds of formula (I), (Ia), (II), (IIa), etc. and mixtures thereof including enantiomers, optical isomers and their racemates (i.e. compounds having an asymmetric carbon);

with respect to reaction time, a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;

and similarly with respect to other parameters such as concentrations, elements, etc.

It is thus to be understood herein for example that a reference to an alkyl group comprising from 1 to 10 carbon atoms includes and specifically refers to an octyl, a straight chain alkyl group of 6 to 10 carbon atoms (e.g. C₆₋₁₀), to a “straight alkyl group of 1 to 6 carbon atoms”, namely, for example, methyl, ethyl, propyl, butyl, pentyl, and hexyl; and so on.

It is further to be understood herein for example that a reference to an alkyl group comprising from 1 to 10 carbon atoms includes and specifically refers to a “branched alkyl group of 3 to 6 carbon atoms”; that a reference to a “branched alkyl group of 3 to 6 carbon atoms” includes for example, without limitation, iso-butyl, tert-butyl, 2-pentyl (i.e. 2-methyl-butyl), 3-pentyl (i.e. 3-methyl-butyl; isopentyl), neopentyl, tert-pentyl, etc; and so on.

It is also to be understood herein, for example that a “cycloalkyl group having 3 to 7 carbon” includes for example, without limitation, cyclopropyl, cyclobutyl, cyclopentyl, cyclohexyl (i.e., C₆H₁₁), etc. ; and so on.

It is also to be understood herein, for example that aryl including heteroaryl includes single ring and fused ring structures; the ring structures may for example comprise up to 14 ring atoms, (e.g. phenyl, pyridyl, pyrimidyl, indolyl, napthyl, etc.) ; and so on.

It is also to be understood herein, for example that phenylalkyl includes benzyl, phenylethyl, phenylpropyl or phenylbutyl.

It is in particular to be understood herein for example that the compound formulae (i.e. formula (I), (Ia), (Ib ), (II), (IIa) etc.) referred to herein, each includes, each and every individual compound (including the isomers thereof) described thereby as well as each and every possible class or sub-group or sub-class of compounds; thus it is to be understood that such individual compounds or classes or sub-classes are inherently defined herein in every and any possible manner whatsoever; it is thus for example to be understood that the definitions herein with respect to any such individual compound, class or sub-class include both positive as well as negative or exclusionary definitions i.e. the definitions herein incorporate any and all definitions that may be worded as positively including particular individual compounds, classes or sub-classes and/or as excluding particular individual compounds, classes or sub-classes or combinations thereof; for example an exclusionary definition for the formulae (e.g. (I), etc.) may read as follows: “provided that when one of R₁ and R₂ is methyl and the other is H, R₈ may not occupy the 2 position”.

As already mentioned, in the present specification, cycloalkyl may for example, include cyclopropyl, cyclobutyl, cyclopentyl, cyclohexyl, or cycloheptyl; phenylalkyl may for example, include benzyl, phenylethyl, phenylpropyl or phenylbutyl; a 5 or 6-membered cycle formed together with the adjacent nitrogen atom may for example, include pyrrolidinyl, piperidino, piperazinyl, morpholino or thiomorpholino; straight chain alkylene may for example, include methylene, ethylene, trimethylene, tetramethylene or pentamethylene; alkylene which is substituted by alkyl may for example, include methylmethylene, methylpropylene, methyltrimethylene, dimethylethylene, ethylethylene or dimethyltrimethylene; alkyl may for example, include methyl, ethyl, propyl, isopropyl, butyl, isobutyl, tert-butyl or octyl.

The compounds according to the present invention include where applicable and desired, pharmaceutically acceptable salts (e.g. pharmaceutically acceptable ammonium salts such as for example acid addition salts). Thus, for example, compounds of the formula (I), (II), (Ia), (IIa), etc., where appropriate and/or desired may be obtained as or converted to pharmaceutically acceptable acid addition salts thereof according to any conventional manner. The acid for forming pharmaceutically acceptable acid addition salts can be suitably selected from inorganic acids (e.g. hydrochloric acid, hydrobromic acid, sulfuric acid, phosphoric acid) and organic acids (e.g. acetic acid, methanesulfonic acid, maleic acid, fumaric acid). These salts can be converted to the corresponding free base according to a conventional manner, for example, by reacting with an alkali such as sodium hydroxide or potassium hydroxide. The compound of the formula (I) (II), (Ia), (IIa), etc., may also when appropriate or desired be converted to a quaternary ammonium salt thereof. If a compound of the formula (I), (II), (Ia), (IIa), etc., is a compound having a carboxyl group as a substituent it may be converted to a salt, such as a salt comprising a metal ion (e.g. sodium, potassium, calcium, aluminum) or amino acid ion (e.g. lysine, omithine). In the case where a compound of the formula (I) (II), (Ia), (IIa), etc., comprises an acid function (e.g. carboxyl group) then where appropriate and/or desired such compound may be obtained as or converted to a salt comprising a pharmaceutically acceptable metal ion (e.g. alkali metal ion or alkaline earth metal ion).

Pharmaceutically acceptable salts of the compounds of this invention include those derived from pharmaceutically acceptable inorganic and organic acids and bases. Examples of such acid salts include: acetate, adipate, alginate, aspartate, benzoate, benzenesulfonate, bisulfate, butyrate, citrate, camphorate, camphorsulfonate, cyclopentanepropionate, digluconate, dodecylhydrogensulfate, dodecylsulfate, ethanesulfonate, formate, fumarate, glucoheptanoate, glycerophosphate, glycollate, hemisulfate, heptanoate, hexanoate, hydrochloride, hydrobromide, hydroiodide, 2-hydroxyethanesulfonate, lactate, maleate, malonate, methanesulfonate, 2-naphthylsulfonate, nicotinate, nitrate, oxalate, palmoate, pectinate, perchlorate, persulfate, 3-phenylpropionate, phosphate, picrate, pivalate, propionate, salicylate, succinate, sulfate, tartrate, thiocyanate, tosylate, and undecanoate.

A compound of this invention, for example a compound of the formula (I) (II), (Ia), (IIa), etc., can comprise a quaternary ammonium group. This invention also envisions the quatemization of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chlorides, bromides and iodides; dialkyl sulfates including dimethyl, diethyl, dibutyl and diamyl sulfates; long chain halides such as decyl, lauryl, myristyl and stearyl chlorides, bromides and iodides, and aralkyl halides including benzyl and phenethyl bromides. Water or oil-soluble or dispersible products may be obtained by such quaternization.

The present invention in particular relates to a compound of formula (I) and pharmaceutically acceptable salts thereof as defined herein selected from the group consisting of

4-(But-3′-en-1′-amino)-N-(4″-pyridyl)cyclohexane Carboxamide

4-(But-3′-en-1′-amino)-N-[2″-(3″′-indolyl)ethyl]cyclohexane Carboxamide

4-(But-3′-en-1′-amino)-N-[(3″-pyridyl)methyl]cyclohexane Carboxamide

4-(But-3′-en-1′-amino)-N-[2″-(2″′-pyridyl)ethyl]cyclohexane Carboxamide

4-(But-3′-en-1′-amino)-N-[4″-(N″-benzyl)piperidyl]-cyclohexane Carboxamide

4-(But-3′-en-1′-amino)-N-(3″-pyridyl)cyclohexane Carboxamide

4-(But-3′-en-1′-amino)-N-(3″-quinolyl)cyclohexane Carboxamide

4-(But-3′-en-1′-amino)-N-(5″-isoquinolyl)cyclohexane Carboxamide

4-(But-3′-en-1′-amino)-N-(6″-quinolyl)cyclohexane Carboxamide

4-(But-3′-en-1′-amino)-N-[4″-(dimethylamino)-benzyl]cyclohexane Carboxamide

4-(But-3′-en-1′-amino)-N-(4″-quinaldyl)cyclohexane Carboxamide

4-(But-3′-en-1′-amino)-N-(5″-indolyl)cyclohexane Carboxamide

4-(But-3′-en-1′-amino)-N-[(4″-pyridyl)methyl]cyclohexane Carboxamide

4-[(N′-methyl)-but-3′-en-1′-amino]-N-(4″-pyridyl)cyclohexane Carboxamide

4-[(N′-benzyl)-but-3′-en-1′-amino]-N-(4″-pyridyl)cyclohexane Carboxamide

4-(But-3′-en-1′-amino)-N-(6″-puryl)cyclohexane Carboxamide

and pharmaceutically acceptable salts thereof.

The present invention in more particularly relates to a compound of formula (I) and pharmaceutically acceptable salts thereof as defined herein selected from the group consisting of

(R,S)-trans-4-(But-3′-en-1′-amino)-N-(4″-pyridyl)cyclohexane Carboxamide,

(R,S)-trans-4-(But-3′-en-1′-amino)-N-[2″-(3′″-indolyl)ethyl]cyclohexane Carboxamide,

(R,S)-trans-4-(But-3′-en-1′-amino)-N-[(3″-pyridyl)methyl]cyclohexane Carboxamide,

(R,S)-trans-4-(But-3′-en-1′-amino)-N-[2″-(2′″-pyridyl)ethyl]cyclohexane Carboxamide,

(R,S)-trans-4-(But-3′-en-1′-amino)-N-[4″-(N″-benzyl)piperidyl]-cyclohexane Carboxamide,

(R,S)-trans-4-(But-3′-en-1′-amino)-N-(3″-pyridyl)cyclohexane Carboxamide,

(R,S)-trans-4-(But-3′-en-1′-amino)-N-(3″-quinolyl)cyclohexane Carboxamide,

(R,S)-trans-4-(But-3′-en-1′-amino)-N-(5″-isoquinolyl)cyclohexane Carboxamide,

(R,S)-trans-4-(But-3′-en-1′-amino)-N-(6″-quinolyl)cyclohexane Carboxamide,

(R,S)-trans-4-(But-3′-en-1′-amino)-N-[4″-(dimethylamino)-benzyl]cyclohexane Carboxamide,

(R,S)-trans-4-(But-3′-en-1′-amino)-N-(4″-quinaldyl)cyclohexane Carboxamide,

(R,S)-trans-4-(But-3′-en-1′-amino)-N-(5″-indolyl)cyclohexane Carboxamide,

(R,S)-trans-4-(But-3′-en-1′-amino)-N-[(4″-pyridyl)methyl]cyclohexane Carboxamide,

(R)-trans-4-(But-3′-en-1′-amino)-N-(4″-pyridyl)cyclohexane Carboxamide,

(S)-trans-4-(But-3′-en-1′-amino)-N-(4″-pyridyl)cyclohexane Carboxamide,

(R,S)-trans-4-[(N′-methyl)-but-3′-en-1′-amino]-N-(4″-pyridyl)cyclohexane Carboxamide,

(R,S)-trans-4-[(N′-benzyl)-but-3′-en-1′-amino]-N-(4″-pyridyl)cyclohexane Carboxamide,

(R,S)-trans-4-(But-3′-en-1′-amino)-N-(6″-puryl)cyclohexane Carboxamide,

and pharmaceutically acceptable salts thereof.

The present invention further relates to a compound of formula (II) and pharmaceutically acceptable salts thereof as defined herein selected from the group consisting of

4-[N′-(Methyl)hydrazino]-N-(4″-pyridyl)cyclohexane Carboxamide

4-[N′-(Propyl)hydrazino]-N-(4″-pyridyl)cyclohexane Carboxamide

4-{N′-[3′-(Methyl)butyl]hydrazino}-N-(4″-pyridyl)cyclohexane Carboxamide

4-{N′-[1′-(Methyl)ethyl]hydrazino}-N-(4″-pyridyl)cyclohexane Carboxamide

4-[N′-(Benzyl)hydrazino]-N-(4″-pyridyl)cyclohexane Carboxamide

4-{N′-[2′-(Phenyl)ethyl]hydrazino}-N-(4′″-pyridyl)cyclohexane Carboxamide

4-{N′-[2′,2′-(Diphenyl)ethyl]hydrazino}-N-(4″-pyridyl)cyclohexane Carboxamide

4-{N′-[4′-(Benzyloxy)benzyl]hydrazino}-N-(4′″-pyridyl)cyclohexane Carboxamide

4-{N′-[(Cyclohexyl)methyl]hydrazino}-N-(4′″-pyridyl)cyclohexane Carboxamide

4-[N′-(Octyl)hydrazino]-N-(4′″-pyridyl)cyclohexane Carboxamide

4-{N′-[3′-(Phenyl)prop-2′-enyl]hydrazino}-N-(4′″-pyridyl)cyclohexane Carboxamide

and pharmaceutically acceptable salts thereof.

The present invention further relates to a compound of formula (II) and pharmaceutically acceptable salts thereof as defined herein selected from the group consisting

cis-4-[N′-(Methyl)hydrazino]-N-(4″-pyridyl)cyclohexane Carboxamide,

trans-4-[N′-(Methyl)hydrazino]-N-(4″-pyridyl)cyclohexane Carboxamide,

trans-4-[N′-(Propyl)hydrazino]-N-(4″-pyridyl)cyclohexane Carboxamide,

trans-4-{N′-[3′-(Methyl)butyl]hydrazino}-N-(4″-pyridyl)cyclohexane Carboxamide,

trans-4-{N′-[1′-(Methyl)ethyl]hydrazino}-N-(4″-pyridyl)cyclohexane Carboxamide Dihydrochloride,

trans-4-[N′-(Benzyl)hydrazino]-N-(4″-pyridyl)cyclohexane Carboxamide,

cis-4-[N′-(Propyl)hydrazino]-N-(4″-pyridyl)cyclohexane Carboxamide,

cis-4-{N′-[3′-(Methyl)butyl]hydrazino}-N-(4″-pyridyl)cyclohexane Carboxamide,

cis-4-{N′-[1′-(Methyl)ethyl]hydrazino}-N-(4″-pyridyl)cyclohexane Carboxamide,

cis-4-[N′-(Benzyl)hydrazino]-N-(4″-pyridyl)cyclohexane Carboxamide,

trans-4-{N′-[2′-(Phenyl)ethyl]hydrazino}-N-(4′″-pyridyl)cyclohexane Carboxamide,

trans-4-{N′-[2′,2′-(Diphenyl)ethyl]hydrazino}-N-(4′″-pyridyl)cyclohexane Carboxamide,

trans-4-{N′-[4′-(Benzyloxy)benzyl]hydrazino}-N-(4′″-pyridyl)cyclohexane Carboxamide,

trans-4-{N′-[(Cyclohexyl)methyl]hydrazino}-N-(4′″-pyridyl)cyclohexane Carboxamide,

trans-4-[N′-(Octyl)hydrazino]-N-(4′″-pyridyl)cyclohexane Carboxamide, 1,4-trans-2′,3′-trans-4-{N′-[3′-(Phenyl)prop-2′-enyl]hydrazino}-N-(4′″-pyridyl)cyclohexane Carboxamide,

and pharmaceutically acceptable salts thereof.

The present invention also provides for a “pharmaceutical composition” comprising a compound in accordance with the present invention (namely, a compound of formula (I) (II), (Ia), (IIa), etc. including pharmaceutically acceptable salts thereof) and a pharmaceutically acceptable carrier. A “pharmaceutical composition” may comprise one or more such compounds of the present invention. It is to be understood herein that the expression “pharmaceutical composition” refers to a composition which comprises a therapeutically effective amount(s) of active agent(s) wherein the active agent comprises a compound in accordance with the present invention, namely a compound of formula (I) (II), (Ia), (IIa), etc. including pharmaceutically acceptable salts thereof. A “therapeutically effective amount” as used herein refers to that amount which provides a therapeutic effect for a given condition and administration regimen.

The term “pharmaceutically acceptable carrier” is to be understood herein as referring to any substance that may, medically, be acceptably administered to a patient, together with a compound of this invention, and which does not undesirably affect the pharmacological activity thereof; a “pharmaceutically acceptable carrier” may thus be a pharmaceutically acceptable member(s) selected from the group comprising or consisting of diluents, preservatives, solubilizers, emulsifiers, adjuvant, tonicity modifying agents, buffers as well as any other physiologically acceptable vehicle.

Such pharmaceutically acceptable carriers include carriers known in the art such as for example, phosphate buffer solution such as 0.01-0.1 M phosphate buffer and preferably 0.05 M phosphate buffer or phosphate buffered saline, and 0.8 % saline solution. Additionally, such pharmaceutically acceptable carriers may be aqueous or non-aqueous solutions, suspensions, and emulsions. Preferably such solutions, suspensions, and emulsions are aqueous. Examples of non-aqueous solvents include propylene glycol, polyethylene glycol, vegetable oils such as olive oil or soybean oil, and pharmaceutically acceptable organic esters such as ethyl oleate which are suitable for use in injectable formulations. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. Formulation of compounds of this invention are preferably performed in the absence of oxygen, such as in an inert atmosphere for example nitrogen or argon. Liquids used in the preparation of formulation compositions of this invention are preferably sparged with an inert gas prior to use to substantially remove unwanted dissolved gases such as air and oxygen.

Additionally such compositions may more particularly be liquids or lyophilized or otherwise dried formulations and include diluents of various buffer content (e.g., Tris-HCl., acetate, phosphate), pH and ionic strength; additives such as albumin or gelatin which can prevent absorption of an active compound of this invention to a surface such as glass, pharmaceutically acceptable detergents (e.g., Tween 20, Tween 80, Pluronic F68, bile acid salts); solubilizing agents (e.g., glycerol, polyethylene glycerol); anti-oxidants (e.g., ascorbic acid, sodium metabisulfite); preservatives (e.g., thimerosal, benzyl alcohol, parabens); bulking substances or tonicity modifiers (e.g., lactose, mannitol). The active agent may for example be associated with liposomes, emulsions, microemulsions, micelles, unilamellar or multilamellar vesicles, erythrocyte ghosts, or spheroplasts. The carrier element of such compositions may be chosen with an eye to influence the physical state, solubility, stability, rate of in vivo release, and rate of in vivo clearance. The compositions of this invention can comprise controlled or sustained release compositions and can comprise a compound of this invention formulated in lipophilic depots (e.g., fatty acids, waxes, oils). The pharmaceutical composition may be formulated so as to able to be administered or for administration to a patient in need of treatment parenterally, paracancerally, transmucosally, transdermally, intramuscularly, intravenously, intradermally, subcutaneously, intraperitonealy, intraventricularly, intracranially intratumorally or more preferably, directly at a central nervous system (CNS) lesion site or a peripheral nervous system (PNS) lesion site. Compositions of this invention that are intended for injectable or implantable use into a mammal are preferably sterilizable, for example by filtration through a membrane or filter intended for such use, by irradiation for example by irradiation derived from a radioisotope or by ultraviolet irradiation, or by thermal sterilization such as by steam sterilization (e.g., at 121° C. for an effective time such as about 15 minutes or more) or by thermal sterilization in the absence of steam. An injectable composition of this invention, preferably comprising an a unit dose amount of a compound of this invention, can be filled into a container such as a vial or a syringe or a pharmaceutically acceptable plastic bag, and preferably under an inert atmosphere such as nitrogen or argon and the like or under a substantially inert atmosphere such as an atmosphere consisting essentially of nitrogen or argon and the like, sealed for example with a stopper and crimp cap for a vial, and sterilized.

In one aspect of this invention, a method of treatment of a mammal can comprise administration by a route selected from parenteral, paracanceral, transmucosal, transdermal, intramuscular, intravenous, intradermal, subcutaneous, intraperitoneal, intraventricular, intracranial, intratumoral, or more preferably, directly at a central nervous system (CNS) lesion site or at a peripheral nervous system (PNS) lesion site, of a compound of this invention in a pharmaceutically acceptable carrier.

Compositions of this invention that are intended for injectable or implantable use into a mammal can comprise a kit of parts. A kit of parts of this invention can comprise two parts, wherein for example, one part of such kit can comprise a dried composition of this invention, for example such as a lyophilized formulation of a compound of this invention, sealed in a first vessel, for example such as vial or a compartment of a syringe, and another part of such kit can consist of a sterile aqueous solution, for example such as sterile water or buffered water, sealed in a second vessel, wherein the aqueous solution in the second vessel can be in an amount suitable for addition to the lyophilized formulation in the first vessel suitable to form an injectable unit dosage form of the compound of this invention, preferably uniformly dissolved or dispersed in the aqueous medium. Transfer of aqueous medium between vessels can be via syringe or cannula or the like and done in a fashion to minimize contamination by ambient microbials. The unit dosage form prepared according to this invention can be administered by injection. Optionally, the kit of parts can comprise a third part which can be a container or a packaging material shaped in a manner suitable to hold the other parts of the kit in proximity prior to and optionally during rehydration or even during administration of the formulation of this invention. The third part of the kit for example can comprise a first socket or cradle of a size suitable to hold, optionally firmly or permanently, the first vessel of the kit, and a second socket or cradle of a size suitable to hold, optionally firmly or permanently, the second vessel of the kit, and can optionally comprise a cannula for use in transfer of the aqueous medium from the second vessel to the first vessel.

The present invention in an additional aspect includes the use of one or more compounds (namely a base as well as a salt thereof) of formula (I), (II), (Ia), (Ia), etc. for the manufacture of a pharmaceutical composition useful for the treatment of a herein mentioned medical condition. The present invention in a further aspect relates to the use of one or more compounds (namely a base as well as a salt thereof) of formula (I), (II), (Ia), (IIa), etc. for the treatment of a herein mentioned medical condition.

It is also to be understood herein that “g” or “gm” is a reference to the gram weight unit; that “C”, or “°C.” is a reference to the Celsius temperature unit; and “psig” is a reference to pounds per square inch guage” “M” is a reference to Molarity.

TABLE 1
Abbreviations

| Abbreviation | Full name |
|---|---|
| ATCC | American Type Cell Culture |
| ECACC | European Collection of Cell Cultures |
| C3 | ADP-ribosyl transferase C3 |
| NGF | Nerve growth factor |
| BDNF | Brain-derived neurotrophic factor |
| C or ° C. | Degree Celcius |
| mL or ml | milliliter |
| μL or μl | microliter |
| μM | micromolar |
| mM | millimolar |
| M | molar |
| N | normal |
| CNS | Central nervous system |
| PNS | Peripheral nervous system |
| HIV | Human immunodeficiency virus |
| kDa | kilodalton |
| GST | Glutathione S-transferase |
| SDS-PAGE | Sodium dodecyl sulfte polyacrylamide gel electrophoresis |
| PBS | Phosphate buffered saline |
| U | unit |
| BBB | Basso, Beattie Breshnahan behavior recovery scale |
| IPTG | Isopropyl D-thiogalactopyranoside |
| rpm | Rotation per minutes |
| DTT | dithiothreitol |
| PMSF | Phenylmethylsulfonyl fluoride |
| NaCl | Sodium chloride |
| MgCl2 | Magnesium chloride |
| HBSS | Hank's balanced salt solution |
| NaOH | Sodium hydroxide |
| CSPG | chondroitin sulfate proteoglycan |
| PKN | Protein kinase N |
| RSV | Rous sarcoma virus |
| HL | Hind limb |
| FL | Fore limb |
| IN-1 | monoclonal antibody called IN-1 |
| ADP | Adenosine di-phosphate |
| ATP | Adenosine tri-phosphate |
| 32P | Isotope 32 of phosphorus |
| DHFR | Dihydrofolate reductase |
| DMSO | Dimethylsulfoxide |
| L | liter |
| BOC | tert-butyloxycarbonyl |
| Et | ethyl |
| Me | methyl |
| R | various functional group |
| P | protecting group |
| Ph | phenyl |
| TEA | triethylamine |
| DIEA | diisopropylethylamine |
| THF | tetrahydrofuran |
| DMC | dichloromethane |
| δ | ppm unit |
| J | coupling constant |
| Hz | Hertz |
| FAB | fast atom bombardment |
| MAB | metastable atom bombardment |
| EtOAc | ethyl acetate |
| NMR | nuclear magnetic resonance |
| s | singlet |
| d | doublet |
| t | triplet |
| m | multiplet |
| HRMS | high resolution mass spectrometry |

Specific compounds are sometimes referred to herein by alphanumeric designations such as, for example, BA-1001, BA-1002, BA-1017 and the like; and/or by a reference (alpha)numeral such as for example, 6, 12a, 38, and the like. The reference (alpha)numerals refer to a compound structure per se (e.g. Compound 26) or are associated with specific compounds by being mentioned with respect to a given compound; alternatively the reference (alpha)numerals refer to a compound structure by being associated with a generic graphic structure, and by being associated with specific definitions of various substituents or groups; for example, the compound 12a is associated a generic structure wherein R¹=H and R²=methyl (Me); 9b is associated a generic structure wherein R¹=CO₂t-Bu (t-Bu=t-butyl) and R²=n-propyl (n-Pr); (See graphics below). On the other hand, alphanumeric designations (such as BA-1001) are associated with specific compounds by being mentioned with respect to a given compound and/or an above mentioned reference (alpha)numeral. Thus for example BA-1008 and 15e are given as alternate designations for the same following compound as follows: (R,S)-trans-4-(But-3′-en-1′-amino)-N-(3″-pyridyl)cyclohexane Carboxamide Dihydrochloride [BA-1008, (R,S)-15e].

Synthetic components and chemical intermediates and reagents useful to prepare compounds of the present invention may, for example, be synthesized by the methods and by following relevant aspects of synthesis schemes as set forth in U.S. Pat. Nos. 4,997,834, 5,478,838 and 6,218,410, which synthesis schemes are necessarily modified to accommodate the functional group manipulations required to achieve the preparation of the compounds of this invention.

Previous syntheses of 1,4-substituted cyclohexane derivatives such as Y-27632 have relied on the use of alpha-alkylbenzylamines as chiral educts which were subsequently acylated under Friedel-Crafts conditions at the para-position and reduced at the aromatic ring to provide the 1,4-substituted cyclohexane system (Arita et al., U.S. Pat. No. 5,478,838; Muro et al., U.S. Pat. No. 4,997,834). One drawback to this approach has been the limited number of enantiomerically enriched alpha-alkylbenzylamines that are available commercially. A second significant problem has been the harsh conditions of the aromatic acylation and reduction chemistry, which conditions do not tolerate the presence of many functional groups such as a double bond or allylic olefinic group. The present invention thus provides by way of example an alternative method useful to prepare and manufacture a 1,4-substituted cyclohexane system. The example method is specifically designed as a stereoselective means for generating analogs possessing a wide diversity of alkyl-branched amine moieties. The example synthesis scheme I shown below is given with generic formulae wherein various functional groups have generic designations such as R¹, R², R³, R⁴, and R⁵; these generic designations may take on any suitable or appropriately designated values as parameters to provide structures in accordance with the present invention as described herein (e.g. R³ and R⁵ may take on values to provide structures in accordance with the present invention, i.e. a compound of formula (I)).

### EXAMPLE SYNTHESIS SCHEME I

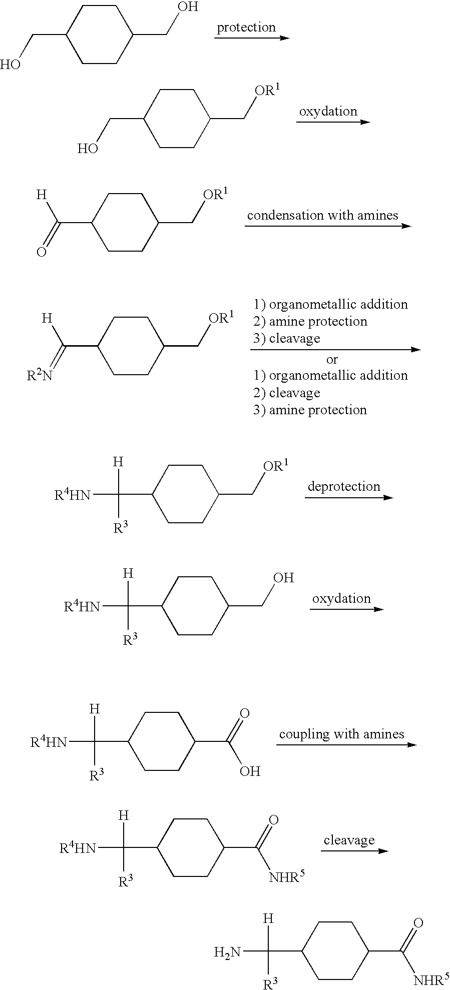

Alternative structures and synthesis schemes are graphically illustrated after the following descriptive passage with respect thereto (i.e. the various compound structures are as mentioned above referred to by reference numbers (or alphanumeric designation) which are set out in the following graphical representation of the compound structures).

Referring to the below illustrated synthesis schemes, bicyclic amino acid analogs 26 and 27 may be synthesized and used to generate compounds of the present invention, which can be used as candidates for studying the influence of the conformation of the alkyl-branched amino moiety with respect to the cyclohexane ring in BA-1003. The 6,5-fused ring amino acid 26 may be generated by Pictet-Spengler reaction of formaldehyde with alpha-methylbenzylamine to provide the isoindoline 28 (Scheme II).³ The carboxylate group may then be installed by Friedel-Crafts acylation with acetyl chloride followed by oxidation with sodium hypochlorite to afford 29.^{1,2} Hydrogenation of the aromatic ring with ruthenium on carbon and amine protection can provide amino acid 30 which may be converted to different amides 26 as previously described for its monocyclic counterpart BA-1003.^{1,2} The Pictet-Spengler and Bischler-Napieralski reactions between 2-aminoethylphenol 30 and aldehydes or acid chlorides respectively may be used to synthesize the partially saturated isoquinoline 32 found in the 6,6-fused ring amino acid 27.³ Conversion of the phenol to an aryl triflate followed by palladium catalyzed carbonylation can be used to synthesize acid 33.4 Reduction of the aromatic ring with ruthenium on carbon can provide amino acid 34 for conversion to different amides 27.

Replacement of the chiral alpha-carbon of BA-1003 with a nitrogen atom provides 39 which can readily isomerize to adopt either configuration, i.e. hydrazine based analogs. They can be assembled from 4-oxo-cyclohexane carboxylate 35 and a suitably protected hydrazine 36 by a reductive amination protocol to give hydrazine 37 (scheme III). Ester hydrolysis can then provide amino acid 38 for conversion to different amides 39. Scheme IIIa is a more particular example of scheme III.

In the previous paragraphs the following references were referred to by respective (superscript) number

1. Arita et al., U.S. Pat. No. 5,478,838.

2. Muro et al., U.S. Pat. No. 4,997,834.

3. Whaley et al., In Organic Reactions; R. Adams, Ed.; Wiley: New York, 1951; Vol. 6, Chapters 2 and 3.

4. (a) Cacchi et al., Tetrahedron Lett. 1985, 1109. b) Cacchi et al., Tetrahedron Lett. 1992, 33, 3939.

The above mentioned compounds 26, 27 and 39 may have the following structures:

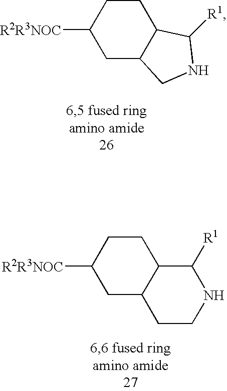

6,5 fused ring 6,6 fused ring

amino amide 26 amino amide 27

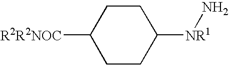

Aza-analogues: 39

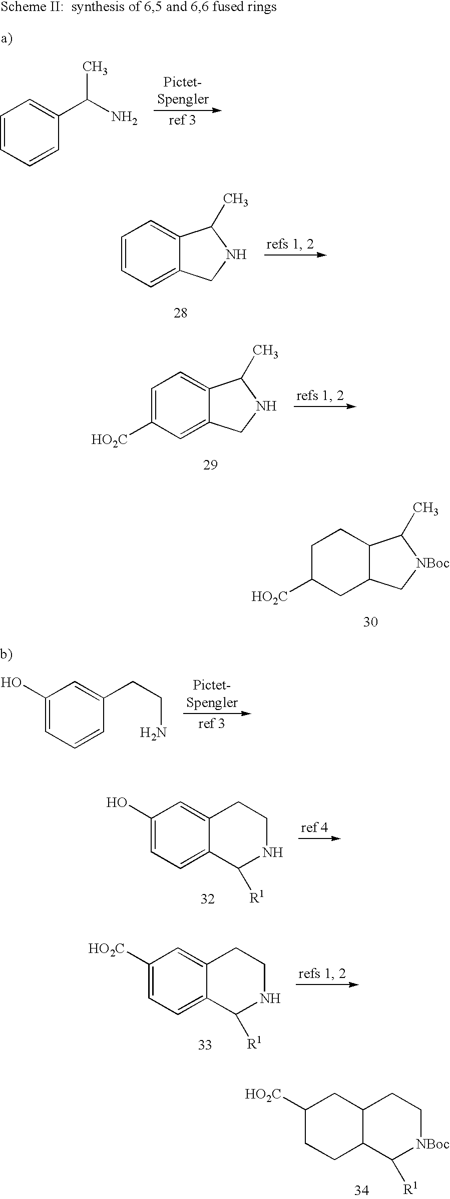

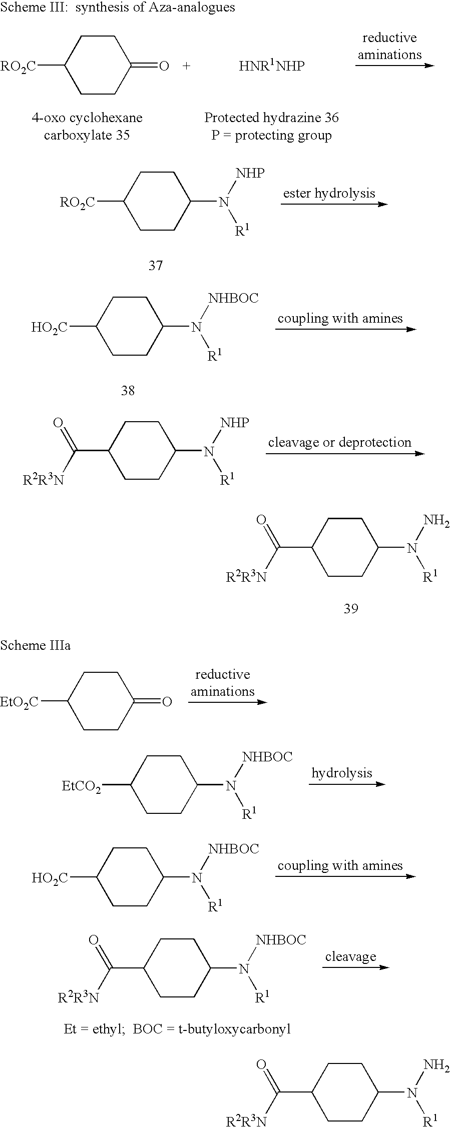

Rho GTPases include members of the Rho, Rac and Cdc42 family of proteins. Our invention concerns kinases that are stimulated by the Rho family members of the Rho class. Rho proteins consist of different variants encoded by different genes. Rho kinase is a well-known target for active Rho, and inactivating Rho kinase has the same effect as inactivating Rho, at least in terms of neurite or axon growth (Kimura and Schubert (1992) Journal of Cell Biology.116:777-783, Keino-Masu, et al. (1996)Cell.87:175-185, Matsui, et al. (1996)EMBO J.15:2208-2216, Matsui, et al. (1998) J. Cell Biol.140:647-657, Ishizaki (1997) FEBS on vehicle include COS cells and CHO cells (ATCC Accession Nos. CRL 1650 and CCL 61, respectively).

As discussed herein, in accordance with the present invention Rho kinase therapeutically active agents may be able to faciliate (for facilitating) axon growth (e.g. regeneration) or prevent apoptosis or cell death, i.e. a compound in accordance with the present invention may be used to inhibit apoptosis, such as following ischemia in the CNS.

In accordance with the present invention a Rho kinase inhibitor compound may be used as a therapeutically active agent for other treatment purposes. A Rho kinase inhibitor can be useful for treatment of a victim of stroke or a victim of a neurodegenerative disease. A Rho signalling pathway is important in repair after stroke (Hitomi, et al. (2000) Life Sci. 67: 1929-39. Trapp et al 2001. Mol.Cell. Neurosci. 17: 883-84). Rho signalling is linked with formation of Alzheimer's disease tangles through its ability to activate PKN which then phosphorylates tau and neurofilaments (Morissette, et al. (2000) Am J Physiol Heart Circ Physiol. 278: H 1769-74., Kawamata, et al. (1998) J. Neurosci. 18: 7402-10., Amano, et al. (1996) Methods Enzymol. 271: 648-50., Watanabe, et al. (1996) Science 271: 645-8.). Rho antagonists can be useful in the treatment of Alzheimer's disease. The new Rho kinase inhibitor drugs can diffuse readily. The new Rho kinase inhibitor drugs may promote repair of nerve cells in diseases that are neurodegenerative. Examples of diseases that are neurodegenerative include, but are not limited to stroke, traumatic brain injury, Parkinson's disease, Alzheimer's disease and ALS. Rho signalling antagonists can be effective in the treatment of other diseases. These include, but are not limited to eye diseases such as glaucoma (Honjo, et al. (2001) Invest. Opthamol. Vis. Sci. 42: 137-44., Rao, et al. (2001) Invest. Opthamol. Vis. Sci.42: 1029-1037.), cancer cell migration and metastasis (Sahai, et al. (1999) Curr. Biol. 9: 136-45., Takamura, et al. (2001) Hepatology 33: 577-81., Imamura, et al. (2000) Jpn J. Cancer Res. 91: 811-6.). The Rho signalling pathway is implicated in smooth muscle relaxation. Rho signalling antagonists can be effective in treatment of hypertension (Chitaley, et al. (2001) Curr Hypertens. Rep. 3: 139-144., Somlyo (1997) Nature 389: 908-911, Uehata, et al. (1997) Nature 389: 990-994), asthma (Nakahara, et al. (2000) Europ. J. Pharmacol. 389: 103-6., Ishizaki, et al.Mol. Pharmacol. (2000) 57: 976-83), and vascular disease (Miyata, et al. (2000) Arterioscler Thromb Vasc Biol. 20: 2351-8., Robertson, et al. (2000) Brit. J. Pharmacol. 131: 5-9.) as well as penile erectile dysfunction (Chitaley, et al. (2001) Nature Med.7: 119-22.). Rho is also important as a cardioprotective protein (Lee et al. 2001. FASEB J. 15:1886-1894).

To test Rho kinase inhibitors for activity, a tissue culture bioassay system was used. This bioassay is used to define activity of Rho kinase inhibitors that will be effective in promoting axon regeneration in spinal cord injury, stroke or neurodegenerative disease. This assay can also detect compounds that are active in stimulating neurite outgrowth by stimulating other parts of signaling pathways important for regulating neurite growth on growth inhibitory substrates.

Neurons do not grow neurites on inhibitory myelin substrates. When neurons are placed on inhibitory substrates in tissue culture, the neurons remain rounded. When an effective Rho kinase inhibitor is added to the neurons, the neurons are able to grow neurites on myelin substrates. The time that it takes for neurons to grow neurites after the addition of a Rho kinase inhibitor is about the same as the time that it takes for neurons to grow neurites if the neurons had been plated on growth permissive substrate such as laminin or polylysine, which time is typically 1 to 2 days in cell culture. An assessment of resulting neurite growth can be scored by visual means. If needed, a quantitative assessment of neurite growth can be performed. This involves measuring after a time such as 1 to 2 days the neurite length a) in control cultures where neurons are plated on myelin substrates and left untreated with Rho kinase inhibitor for such time, b) in positive control cultures, wherein for example neurons are plated on polylysine but left untreated with Rho kinase inhibitor for such time, and c) in cultures analogous to a) and b) that are treated with different concentrations of a Rho kinase inhibitor for such time. A rapid assay can also be used to assess the ability of a Rho kinase inhibitor to promote neurite outgrowth. In this assay, NG108 cells are plated on plastic in the presence or absence of the test substance (a Rho kinase inhibitor). An effective Rho kinase inhibitor will promote more rapid neurite outgrowth than a less effective Rho kinase inhibitor. An ineffective Rho kinase inhibitor will not promote neurite outgrowth. The relative efficacy can be assessed by fixing the cultures 5 hours after plating, and counting the number of cells that have grown neurites.

Rho kinase inhibitors differ from growth factors in their ability to promote neurite outgrowth. Growth factors, such as nerve growth factor (NGF) are not able to overcome growth inhibition by myelin (Lehmann et al, 1999. J.Neurosci. 19: 7537-7547; Jin & Strittmatter, 1997. J. Neurosci. 17: 6256-6263). Our tissue culture experiments are all performed in the presence of the growth factor BDNF for retinal ganglion cells, or NGF for PC-12 cells, or cAMP for NG 108 cells. When growth factors have been tested in vivo, typically they can transiently prevent apoptosis, but they do not promote robust regeneration. This is because they are unable to promote neurite growth on growth inhibitory substates.

A compound can be confirmed as a Rho kinase inhibitor in one of the following ways:

a) Recombinant Rho kinase tagged with myc epitope tag, or a GST tag or any suitable tag is expressed in Hela cells or another suitable cell type by transfection;

b) The kinase is purified from cell homogenates by immunoprecipation using antibodies directed against the specific tag (e.g., myc tag or the GST tag); (purified Rho kinase may alternatively be purchased from Upstate Biotechnology Inc.)

c) The recovered immunoprecipitates of Rho kinase from b) are incubated with [32P] ATP and histone type 2 as a substrate in the presence or absence of the Rho kinase inhibitor. In the absence of Rho kinase inhibitor activity, the Rho kinase phosphorylates histone. In the presence of Rho kinse inhibitor the phosphorylation activity of Rho kinase (i.e. phosphorylation of histone) is blocked, and as such identifies the compound as a Rho kinase antagonist.

Rho kinase inhibution may be determined by use of any other known procedures (i.e. commercial screening methods).

Rho kinase antagonists may be used to treat spinal cord injury to promote functional repair of damaged nerve structures.

Rho kinase antagonists may be used to treat neurodegenerative diseases such as Alzheimer's disease and Parkinson's disease where penetration of the drug to the affected neuronal population can be required for effective treatment. The Rho kinase inhibitors will also be of benefit for the treatment of stroke and traumatic brain injury. Rho kinase antagonists can be useful in the treatment of cancer, for example by mitigating or preventing or reducing cancer cell migration. Rho kinase antagonists are also useful in the treatment of disease involving smooth muscle, such as vascular disease, hypertension, asthma, and penile dysfunction.

For treatment of spinal cord injury, the Rho kinase inhibitor may be used in conjunction with cell transplantation. Many different cell transplants have been extensively tested for their potential to promote regeneration and repair, including, but not restricted to, Schwann cells, fibroblasts modified to express growth factors, fetal spinal cord transplants, macrophages, embryonic or adult stem cells, and olfactory ensheathing glia. Rho kinase inhibitors may be used in conjunction with neurotrophins, apoptosis inhibitors, or other agents that prevent cell death. They may be used in conjunction with cell adhesion molecules such as L1, laminin, and artifical growth matrices that promote axon growth. Rho kinase inhibitors of the present invention may also be used in conjunction with the use of antibodies that block growth inhibitory protein substrates to promote axon growth. Examples of such antibody methods are the use of IN-1 or related antibodies (Schnell and Schwab (1990) 343: 269-272) or through the use of therapeutic vaccine approaches (Huang (1999) 24: 639-647).

The present invention in an aspect relates to pharmaceutical compositions containing, as an active ingredient, a 4-amino(alkyl)cyclohexane-1-carboxamide compound of (I) (II) etc., an isomer thereof or a pharmaceutically acceptable salt (e.g. acid addition salt) thereof; to antihypertensive agents containing, as an active ingredient, a 4-amino(alkyl)cyclohexane-1-carboxamide compound of (I) (II) etc., an isomer thereof or a pharmaceutically acceptable salt (e.g. acid addition salt) thereof; to therapeutic agents for angina pectoris, containing, as an active ingredient, a 4-amino(alkyl)cyclohexane-1-carboxamide compound of (I) (II) etc., an isomer thereof or a pharmaceutically acceptable salt (e.g. acid addition salt) thereof; to therapeutic agents for asthma, containing, as an active ingredient, a 4-amino(alkyl)cyclohexane-1-carboxamide compound of (I) (II) etc., an isomer thereof or a pharmaceutically acceptable salt (e.g. acid addition salt) thereof; to agents for improving peripheral circulation, containing, as an active ingredient, a 4-amino(alkyl)cyclohexane-1-carboxamide compound of (I) (II) etc., an isomer thereof or a pharmaceutically acceptable salt (e.g. acid addition salt) thereof; and the like.

The Compound (I), (II), etc., isomers thereof and pharmaceutically acceptable salts thereof of the present invention may have coronary and cerebral blood flow increasing action as well as renal and peripheral artery blood flow increasing action. The blood flow increasing action can last over a long period of time and antihypertensive action is very strong.

Accordingly, a compound of the present invention may be useful as an antihypertensive agent and as an agent for the prevention and treatment of diseases in circulatory organs such as in treatment of diseases in coronary, cerebral, renal and peripheral arteries.

When the compounds (I), (II), etc., of the present invention are used as medicines, an effective amount thereof is usually admixed with pharmacologically acceptable additives such as excipients, carriers and diluents etc.; thus the active compound may, for example, be orally or parenterally administered in the form of tablet, granule, powder, capsule, injection, ointment, aerosol (e.g. nasal) or suppository.

The dosage will of course vary depending on age, body weight, symptom and disease state of a patient, and a daily dose for a human adult may be formulated for oral administration at single dose or several times divided doses. The concentrations of the compounds described herein in a therapeutic composition will vary depending upon a number of factors, including the dosage of the drug to be administered, the chemical characteristics (e.g., hydrophobicity) of the compounds employed, and the route of administration. In general terms, the compounds of this invention may be provided in an aqueous physiological buffer solution containing about 0.1 to about 10% w/v of compound of this invention for use in parenteral administration. A preferred dose range is from about 0.01 mg/kg to 100 mg/kg of body weight per day. The preferred dosage of drug to be administered is likely to depend on such variables as the type and extent of progression of the neurological or oncology disease, and the overall health status of the particular patient, the relative biological efficacy of the compound selected, the formulation of the compound excipients, and its route of administration.

It has been proposed that Rho is activated upon receipt of signals from various cell membrane receptors and the activated Rho functions as a molecule switch of a broad range of cell phenomena, such as smooth muscle contraction, cell motility, cell adhesion, morphological changes of cell, cell growth and the like, via actomyosin system.

C3 enzyme and analogues of C3 can inhibit the actions of Rho. These are proteins cannot readily permeate cytoplasm, which can reduce their utility in development for pharmaceutical use. Inhibition of Rho kinase, present downstream of the signal transduction pathway via Rho, is considered to lead to the inhibition of responses of various cell phenomena due to Rho. Thus it would be advantageous to have available other types of specific inhibitors of Rho kinase.

A Rho kinase inhibitor can be an effective agent for the prophylaxis and/or treatment of the above-mentioned diseases and phenomena relating to Rho, such as hypertension, angina pectoris, cerebrovascular contraction, asthma, peripheral circulation disorder, immature birth, arteriosclerosis, cancer, inflammation, immune disease, autoimmune disease, AIDS, fertilization and nidation of fertilzed egg, osteoporosis, retinopathy, brain function disorder, bacterial infection of digestive tract and the like.

The present invention in an aspect relates to the provision of a compound that may act as a Rho kinase inhibitor. A Rho kinase inhibitor can exhibit an antihypertensive action, an anti-angina pectoris action, a cerebrovascular contraction suppressive action, an anti-asthma action, a peripheral circulation improving action, an immature birth preventive action, an anti-arteriosclerosis action, an anti-cancer action, an antiinflammatory action, an immunosuppressive action, an autoimmune disease improving action, an anti-AIDS action, a preventive action on fertilization and nidation of fertilized egg, an osteoporosis treating action, a retinopathy treating action, a brain function improving action, a preventive action on bacterial infection of digestive tract. A Rho kinase inhibitor can be useful as a pharmaceutical agent, particularly as a therapeutic agent of hypertension, a therapeutic agent of angina pectoris, a suppressive agent of cerebrovascular contraction, a therapeutic agent of asthma, a therapeutic agent of peripheral circulation disorder, a prophylactic agent of immature birth, a therapeutic agent of arteriosclerosis, an anti cancer drug, an anti-inflammatory agent, an immunosuppressant, a therapeutic agent of autoimmune disease, an anti-AIDS drug, a therapeutic agent of osteoporosis, a therapeutic agent of retinopathy, a brain function improving drug, a contraceptive and a prophylactic agent of digestive tract infection.

A compound which inhibits Rho kinase can be useful as a reagent for the study of Rho and Rho kinase and as a diagnostic of the diseases relating to those, which resulted in the completion of the present invention.

Accordingly, the present invention proposes the following:

A pharmaceutical agent containing a compound of the present invention;

A pharmaceutical agent comprising a compound of the present invention, which is at least one member selected from the group consisting of a therapeutic agent useful for treatment of a spinal cord injury, a stroke, a neurodegenerative disease, glaucoma, hypertension, angina pectoris, a cerebrovascular abnormality wherein the agent is a suppressive agent of cerebrovascular contraction, asthma, a peripheral circulation disorder, arteriosclerosis, a cancer wherein the agent is an anti-cancer drug, inflammation wherein the agent is an anti-inflammatory agent, a disease or condition relating to a tissue or organ implantation or graft wherein the agent is an immunosuppressant, an autoimmune disease, AIDS wherein the agent is an anti-AIDS or anti-HIV drug, osteoporosis, retinopathy, functional abnormalities of the brain wherein the agent is a brain-function-improving drug, immature birth wherein the agent is a prophylactic agent of immature birth, a contraceptive agent wherein the agent is useful for prevention or reversal of nidation of a fertilized egg, and a prophylactic agent useful in the treatment of an infection of the digestive tract;

A pharmaceutical composition containing a therapeutically effective amount of a compound of the present invention and as desired a pharmaceutically acceptable additive;

A reagent containing a compound of the present invention;

A diagnostic containing a compound of the present invention;

A pharmaceutical agent containing a compound of the formula (I), (II), etc., an isomer thereof, and/or a pharmaceutically acceptable salt thereof, which is a therapeutic agent of at least one disease selected from the group consisting of hypertension, angina pectoris, cerebrovascular contraction, asthma and peripheral circulation disorder, which disease is related to Rho kinase activity;

A pharmaceutical agent containing a compound of the formula (I), (II), etc., an isomer thereof, and/or a pharmaceutically acceptable acid addition salt thereof, which is at least one therapeutic agent selected from the group consisting of a therapeutic agent of arteriosclerosis, an anti-cancer drug, an anti-inflammatory agent, an immunosuppressant, a therapeutic agent of autoimmune disease, an anti-AIDS drug, a therapeutic agent of osteoporosis, a therapeutic agent of retinopathy, a brain function improving drug, a prophylactic agent of immature birth, a contraceptive and a prophylactic agent of digestive tract infection;

A reagent having a Rho kinase inhibitory activity, which contains a compound of the formula (I), (II), etc., an isomer thereof and/or a pharmaceutically acceptable salt thereof;

A diagnostic of a disease caused by Rho kinase, which contains a compound of the formula (I), (II), etc., an isomer thereof and/or a pharmaceutically acceptable acid addition salt thereof;

A pharmaceutical agent comprising a compound of the formula (I), (II), etc., an isomer thereof and/or a pharmaceutically acceptable acid addition salt thereof, which agent is a therapeutic agent useful for the treatment of at least one disease selected from the group consisting of hypertension, angina pectoris, cerebrovascular contraction, asthma, inflammation, and brain function disorder, which are caused by Rho kinase;

A pharmaceutical agent comprising a compound of the formula (I), (II), etc., an isomer thereof and/or a pharmaceutically acceptable acid addition salt thereof, which is at least one therapeutic agent selected from the group consisting of a therapeutic agent of peripheral circulation disorder, a therapeutic agent of arteriosclerosis, an anti-cancer drug, an immunosuppressant, a therapeutic agent of autoimmune disease, an anti-AIDS drug, a therapeutic agent of osteoporosis, a therapeutic agent of retinopathy, a prophylactic agent of immature birth, a contraceptive and a prophylactic agent of digestive tract infection;

A reagent having a Rho kinase inhibitory activity, which comprises a compound of the formula (I), (II), etc., an isomer thereof and/or a pharmaceutically acceptable acid addition salt thereof;

A diagnostic for a disease caused by Rho kinase, which diagnostic comprises a compound of the formula (I), (II), etc., an isomer thereof and/or a pharmaceutically acceptable acid addition salt thereof;

A method for treating a disease based on inhibition of Rho kinase, comprising administering a pharmaceutically effective amount of a Compound of the present invention to a patient;

A treating method wherein a disease treatable by the inhibition of the Rho kinase is at least one disease selected from the group consisting of hypertension, angina pectoris, cerebrovascular contraction, asthma, a peripheral circulation disorder, arteriosclerosis, cancer, an inflammation, an immune disease, an autoimmune disease, AIDS, osteoporosis, retinopathy, a brain function disorder, immature birth, fertilization and nidation of fertilized egg and infection of digestive tract;

A method for treating at least one disease selected from the group consisting of hypertension, angina pectoris, cerebrovascular contraction, asthma and a peripheral circulation disorder, which are caused by Rho kinase, and arteriosclerosis, cancer, inflammation, immune disease, autoimmune disease, AIDS, osteoporosis, retinopathy, brain function disorder, immature birth, fertilization and nidation of fertilized egg and infection of digestive tract, which comprises administering a pharmaceutically effective amount of a compound of the formula (I), (II), etc., an isomer thereof and/or a pharmaceutically acceptable salt thereof;

A method for treating at least one disease selected from the group consisting of hypertension, angina pectoris, cerebrovascular contraction, asthma, inflammation and brain function disorder, which are caused by Rho kinase, and a peripheral circulation disorder, arteriosclerosis, cancer, immune disease, autoimmune disease, AIDS, osteoporosis, retinopathy, immature birth, fertilization and nidation of fertilized egg and infection of digestive tract, which comprises administering a pharmaceutically effective amount of a compound of the formula (I), (II), an isomer thereof and/or a pharmaceutically acceptable salt thereof;

A method for treating at least one disease selected from the group consisting of hypertension, angina pectoris, cerebrovascular contraction, asthma, peripheral circulation disorder, arteriosclerosis, cancer, inflammation, immune disease, autoimmune disease, AIDS, osteoporosis, retinopathy, brain function disorder, immature birth, fertilization and nidation of fertilized egg and infection of digestive tract, which comprises administering a pharmaceutically effective amount of a compound of the formula (I), (II), etc., an isomer thereof and/or a pharmaceutically acceptable salt thereof;

Use of a compound of the present invention, for the production of a therapeutic agent of a disease treatable by inhibiting Rho kinase;

The use of a compound of the present invention, wherein the disease treatable by the inhibition of Rho kinase is at least one member selected from the group consisting of hypertension, angina pectoris, cerebrovascular contraction, asthma, peripheral circulation disorder, arteriosclerosis, cancer, inflammation, immune disease, autoimmune disease, AIDS, osteoporosis, retinopathy, brain function disorder, immature birth, feritilization and nidation of fertilized egg and infection of digestive tract; and

The use of a compound of the formula (I), (II), etc.,an isomer thereof and/or a pharmaceutically acceptable salt thereof for the production of a therapeutic agent of at least one disease selected from the group consisting of hypertension, angina pectoris, cerebrovascular contraction, asthma and peripheral circulation disorder caused by Rho kinase, and arteriosclerosis, cancer, inflammation, immune disease, autoimmune disease, AIDS, osteoporosis, retinopathy, brain function disorder, immature birth, fertilization and nidation of fertilzed egg and infection of digestive tract.

The present invention particularly proposes the exploitation of compounds as described herein for use (i.e. methods/compositions/diagnostics, etc.) in relation to

Spinal cord injury and stroke, and traumatic brain injury

cell survival in the retina (relevant to glaucoma, macular degeneratation, and eye disease)

peripheral nerve regeneration: relevant to speeding up the rate of regeneration after surgery,

diabetic neuropathy

neurodegenerative disease (ALS, Alzheimers, Parkinson)

cancer

hypertension and cardiovascular disease, including coated stents

vascular diseases, thrombosis

improved outcome in transpalnatation and surgery

etc.

While the present invention is explained in more detail by the following examples, these examples are not to be construed as limiting the present invention.

### BRIEF DESCRIPTION OF THE FIGURES

In drawings which illustrate example embodiments of the present invention:

FIG. 1 relates to Bioassays of neurite growth;

FIG. 2 relates to Comparison of BA-1003 with the purified stereoisomers, BA-1016 and BA-1017

FIG. 3 illustrates inhibition of ROCKII activity by BA-1016 and BA-1017.

FIG. 4 is a graphic illustration of experiments testing the ability of BA1003, BA10016 and BA-1017 to overcome growth inhibition by MAG

FIG. 5 illustrates a longitudinal section of an optic nerve treated with BA-1016.; and

FIG. 6 illustrates a longitudinal section of a control optic nerve.

FIG. 7 relates to bioassays of neurite growth

FIG. 8 illustrates the IC 50 curve for BA-1016 tested for ROCKII(h) (human Rho kinase) inhibition.

FIG. 9 illustrates the IC 50 curve for BA-1037 tested for ROCKII(h) (human Rho kinase) inhibition.

FIG. 10 illustrates a comparison of bioassay results, rho kinase inhibition, and GSK B kinase activity for compounds tested at a concentration of 10 uM.

FIG. 11 illustrates the anti-proliferative effect of BA-1037 for SK-MEL-1 human malignant melanoma

and

FIG. 12 illustrates the anti-proliferative effect of BA-1037 for Hec 1B human adenocarcinoma cells.

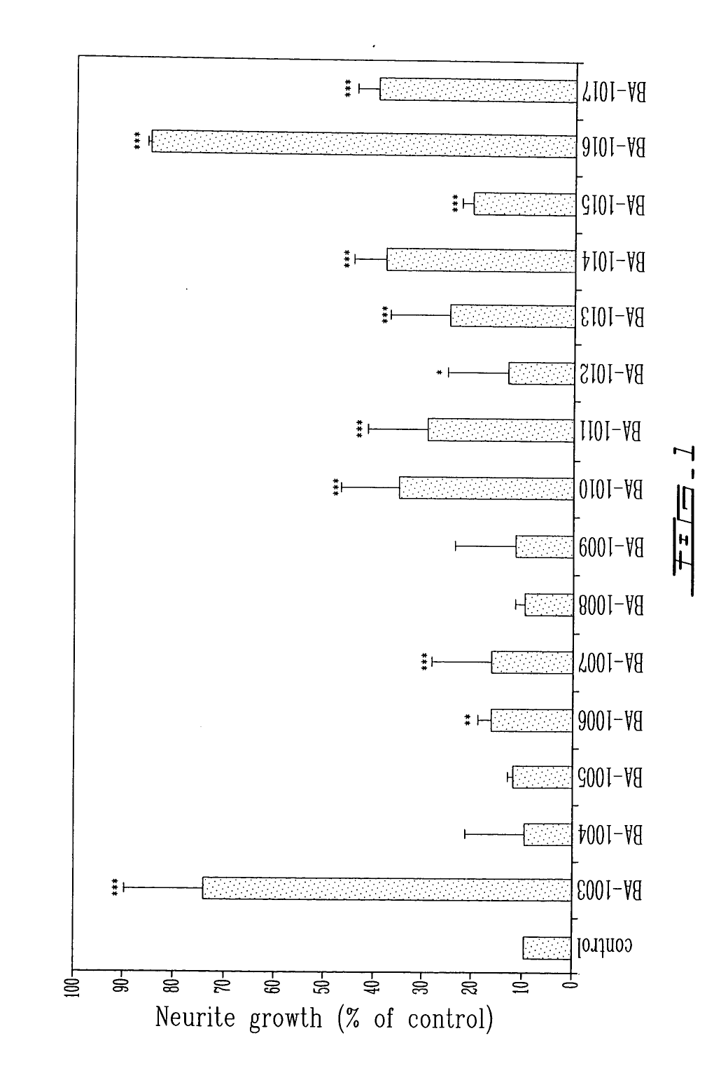

FIG. 1 relates to Bioassays of neurite growth comparing the efficacy of the different test compounds added to the culture medium at a concentration of 35 μM. Two experiments, each in duplicate were performed. The control sample was treated with vehicle alone. Neurite growth was determined as a percent of the control. Values are expressed as mean +/− Standard error of the mean (SEM). Statistical evaluation was with non-normalized data by paired T-test; *P<0.05; **P<0.01; ***P<0.001.

As may be seen from FIG. 1 compounds of the present invention improve neurite growth, for example by an amount of neurite growth that is between about 2 to 8 fold greater than the amount of neurite growth observed in the control in the absence of such compounds.

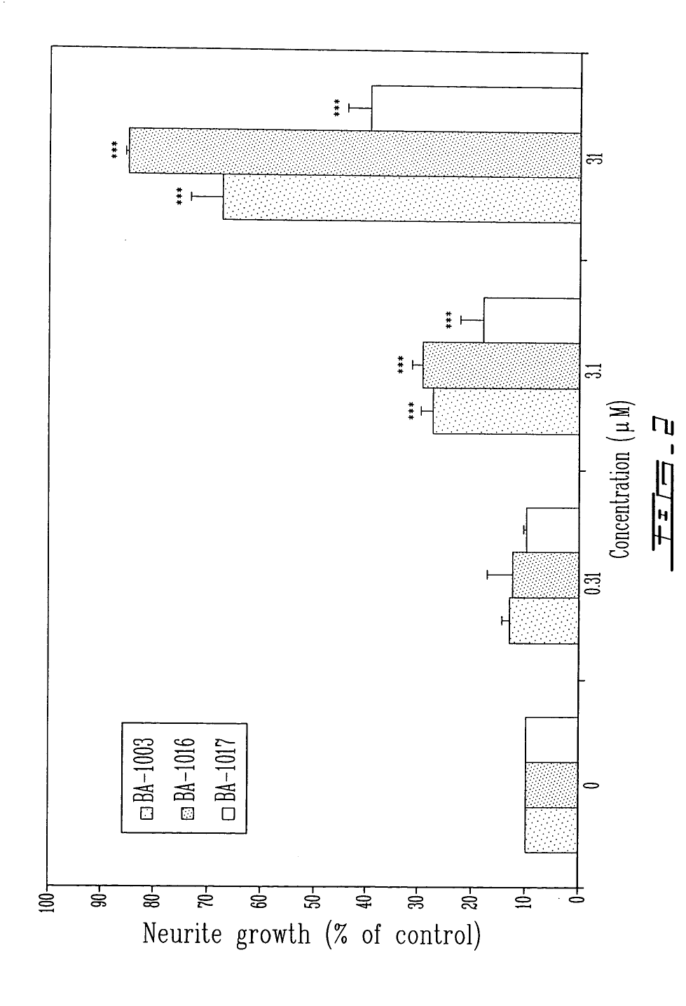

FIG. 2 illustrates a Comparison of BA-1 003 with the enriched sterioisomers, BA-1016 and BA-1017. Referring to FIG. 2, NG108 cells were plated in 96 well plates. Rho kinase inhibitor was added to the tissue culture medium at concentration from 3.1 to 31 μM; as seen the tested compounds provided improved neurite outgrowth, for example by a factor of about 7-fold increase in the amount of neurite growth. This Figure indicates that the compounds tested (i.e. BA-1003, BA-1016 and BA-1017) stimulate axon regeneration, i.e. promote neurite outgrowth. While both stereoisomers promote neurite growth, BA-1016 has better efficacy. Statistics were with non-normalized data (not shown) by paired T-test; ***p<*0.001;

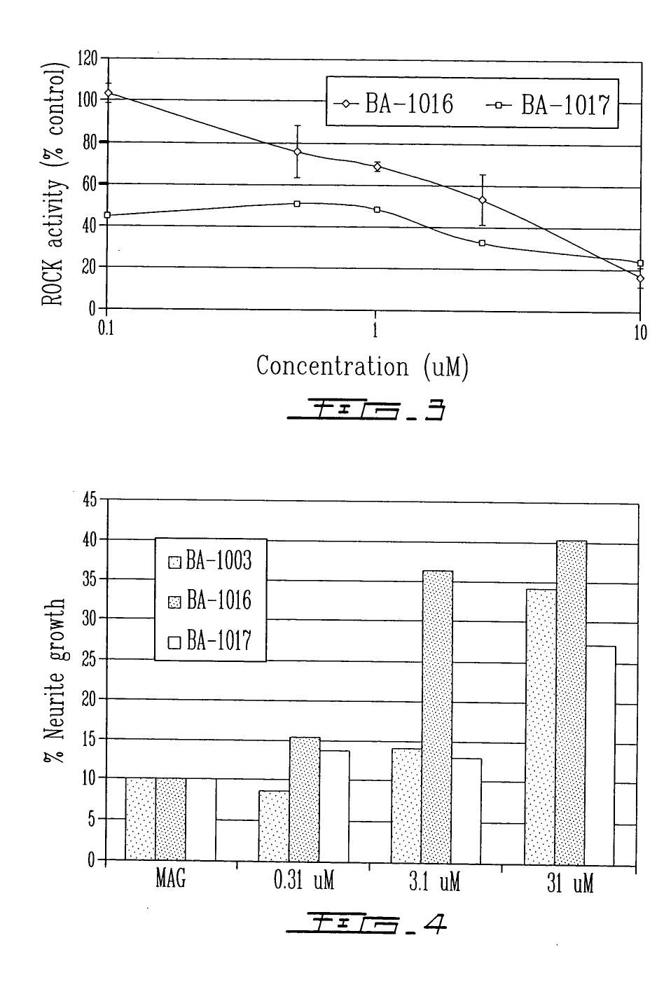

Experiments shown in FIG. 3 are indicative that compounds of the present inhibit Rho kinase. However, an estimated 500 protein kinases are encoded by the human genome. It has been shown, for example, that the Rho kinase Y227632 also inhibits PRK2, MSK1, MAPKAP-K1b AND PHK. (Davies et al. (2000) Biochem. J. 351:95-105). It is thought that each kinase can phosphorylate an average of 30 proteins, therefore, the activity profile is also dependent on the substrate used for the assay. It is possible to test compounds for inhibition of the following human (h) and rat kinases, wherein the compounds can be inhibitors of kinase activity:

CDK5/p35 (h)

JNK1al (h)

MAPK1 (h)

PKA (b)

PKCa (h)

PRK2 (h)

ROCK-II (rat)

GSK3b (h)

PKBa (h)

Fyn (h)

FIG. 4 shows that compounds BA-1003, BA-1016 and BA-1017 can promote neurite outgrowth when neurons are plated on inhibitory MAG substrates. Cells plated on MAG alone do not grow long neurites, wherease addition of 0.31 uM BA-1016 or BA-1017 to such cells allows better neurite outgrowth. Neurons grow long neurites at higher concentrations of 3.1 or 31 μM.

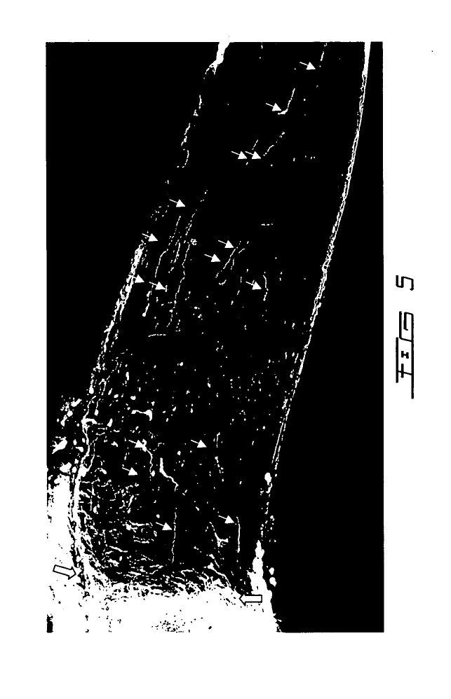

FIGS. 5 shows that BA-1016 can promote axon regeneration after optic nerve injury in adult rats.

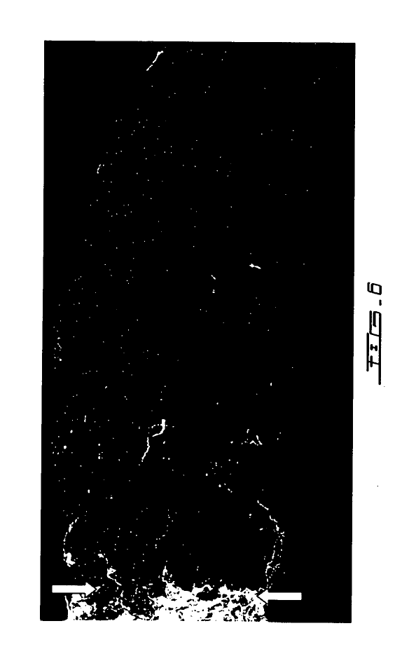

FIG. 6 shows that retinal ganglion cell axons do not regenerate after optic nerve injury without treatment. Treatment of retinal ganglion cells after optic nerve injury with a composition of this invention can induce regeneration of retinal ganglion cell axons.

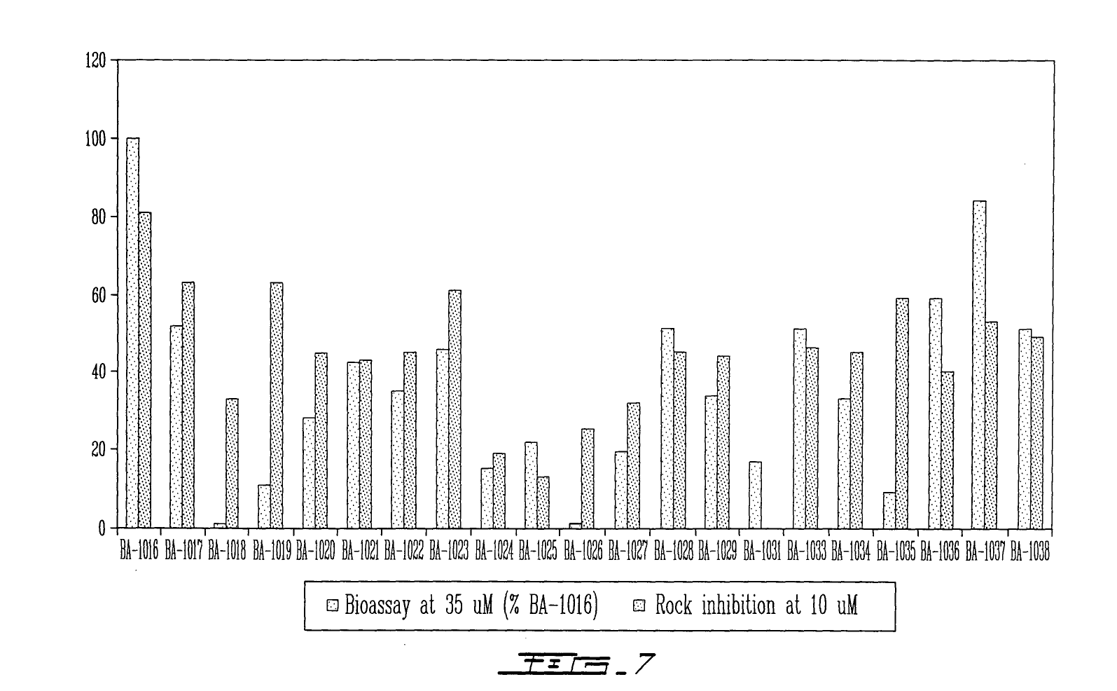

FIG. 7 compares by Bioassay (light grey bars) the ability of compounds BA-1017 to BA-1038 to promote neurite growth when tested at 35 uM. The bioassay values are normalized to BA-1016. BA-1037 promotes neurite outgrowth equally well as BA-1016. The dark bars show the Rho kinase inhibition values when tested at 10 μM. Some of the compounds perform relatively poorly on the neurite outgrowth assay despite good Rho kinase inhibition, perhaps as a function of the relative abilities of the compounds to penetrate living cells.

FIG. 8 shows that BA-1016 has an IC50 of 1.9 μM for human Rho kinase II (ROCK-II (h)).

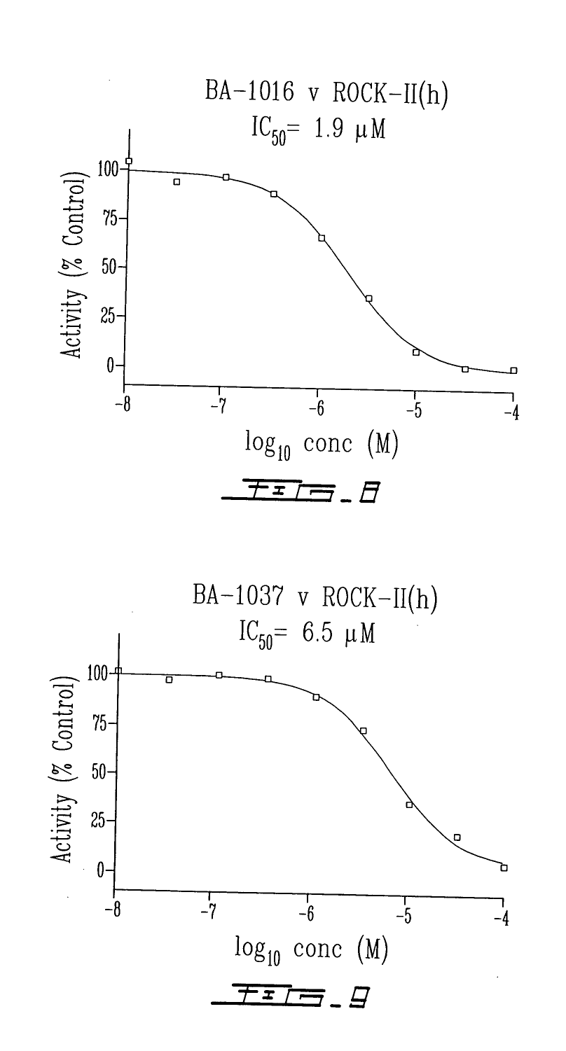

FIG. 9 shows that BA-1037 has an IC50 of 6.5 μM for human Rho kinase II (ROCK-II (h)).

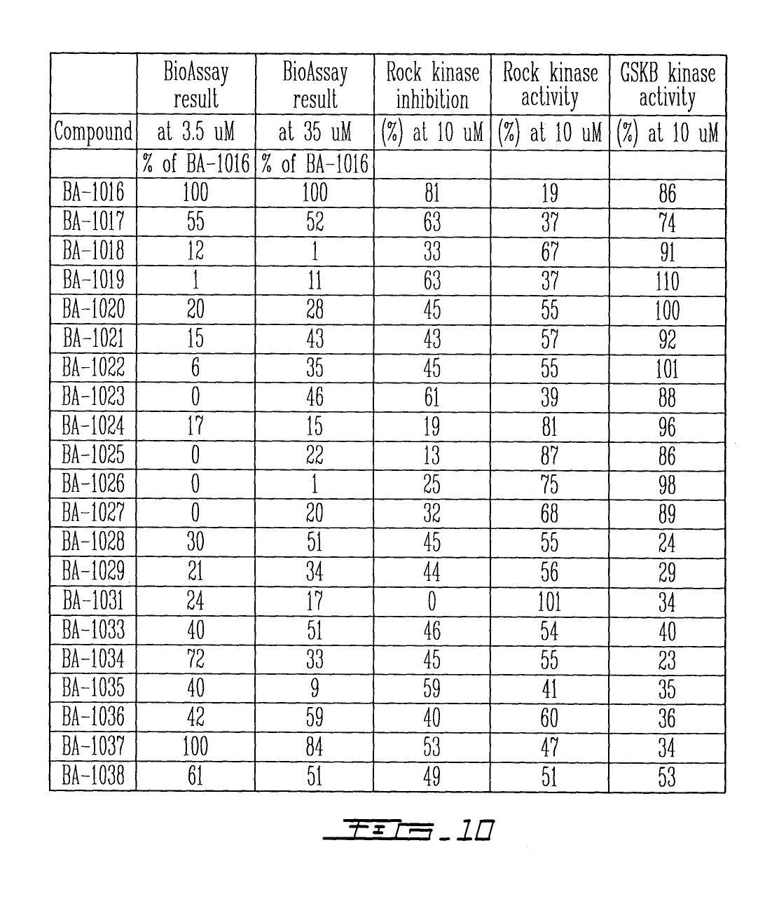

FIG. 10 show a summary of results for compounds BA-1016 to BA-1037. The last column shows that some of the compounds (i.e., BA-1028, BA10,29, BA1-34, BA-1-35, BA-1036, BA-1037, BA-1038) can exert activity towards glycogen synthatase kinase 3 beta (GSKB), a kinase involved in the regulation of apoptosis.

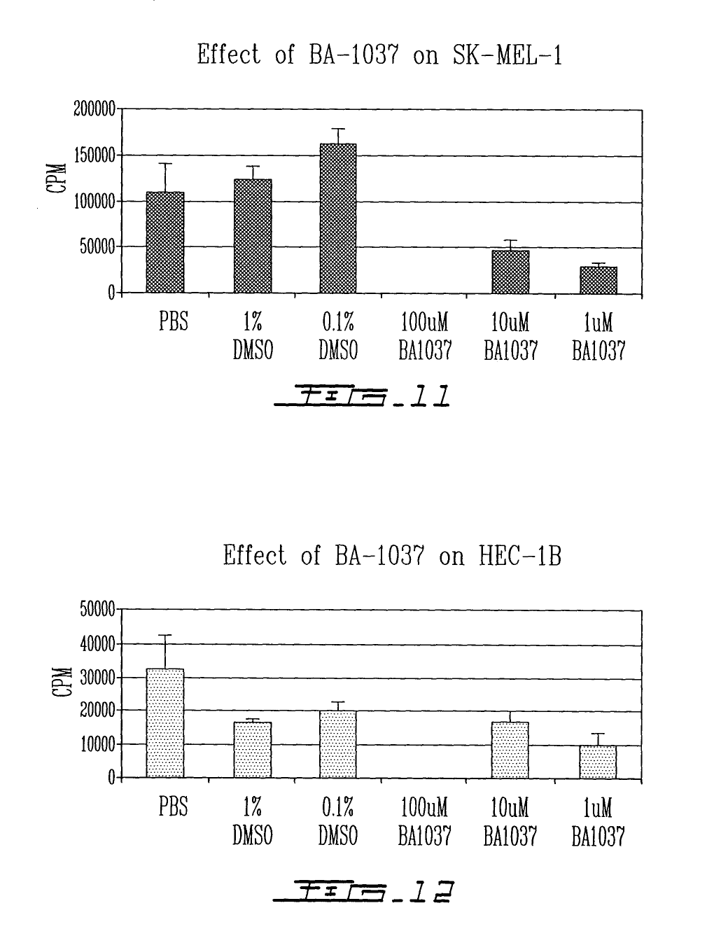

FIG. 11 shows the effect of BA-1037 on human malignant melanocarcinoma cells. All three doses tested significantly blocked cell proliferation compared to vehicle (DMSO) and PBS controls.

FIG. 12 shows the effect of BA-1037 on human malignant HEC 1B cells . The highest dose tested significantly blocked cell proliferation compared to vehicle (DMSO) and PBS controls.

The following graphic figures illustrate compounds of the present invention as well as compounds used and/or made for the synthesis of compounds made in accordance with the more specific compound synthesis description which is set forth below with respect to the examples. The compounds are illustrated in relation to a respective general formula wherein various functional groups have the generic designations such as for example R, R¹, R², and R³; these generic designations may take on the specified values (e.g. R¹ and R₂ may take on values to provide structures in accordance with the present invention, i.e. a compound of formula (I), (II), etc.). The compounds are identified by name or by the use of the above mentioned respective reference (alpha)numeral (e.g. 4a, 13, 16, 17 etc.); each reference name or (alpha)numeral is associated with a definition for the appropriate generic designations such as R, R¹, R₂, and R₃ in order to specify the compound structure associated with name or (alpha)numeral. Thus, for example, for compound 13, R¹, R², and R³ are respectively defined as methoxycarbonyl, H and allyl; for compound 12c, R¹ and R₂ are respectively defined as H and allyl; and so on for the other designated compounds. The illustrated compounds are as may be understood referred to in the following examples, inter alia, by use of the above mentioned respective reference (alpha)numeral (e.g. 4a, 13, 16, 17 etc.).

The following expressions have the following meaning herein

Me=methyl

t-Butyl=tert-butyl

Ph=phenyl

n-Pr=n-propyl

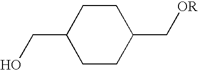

1,4-cyclohexyldimethanol: R═H

2: R═Si(Me)₂t-Bu

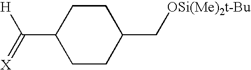

3: X═O

4a: X═NCH(Me)Ph

4b: X═NCH₂Ph

4c: X═

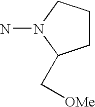

4d: X═NCH₃

5: X═NR¹, where R¹ is a chiral auxilliary

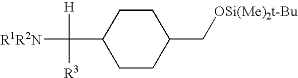

6a: R¹═CH(CH₃)Ph, R²═H, R³═CH₃

6b: R¹═CH(CH₃)Ph, R²═H, R³═CH₂CH═CH₂

6c: R¹═chiral auxilliary, R²═H, R³═CH₂CH═CH₂

6d: R¹═CH₂Ph, R²═H, R³═CH₂CH═CH₂

7a: R¹═R²═H, R³═Me

7b: R¹═R²═H, R³n-Pr

7c: R¹═R²═H, R³═CH₂CH═CH₂

8a: R¹═CO₂t-Bu, R²═H, R³═Me

8b: R¹═CO₂t-Bu, R²═H, R³═CH₂CH₂CH₃

8c: R¹═CO₂t-Bu, R²═H, R³═CH₂CH═CH₂

8d: R¹═CO₂Me, R²═CH₂Ph, R³═CH₂CH═CH₂

8e: R¹═CO₂Me,

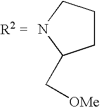

R³═CH₂CH═CH₂

8f: R¹═CO₂t-Bu, R²═CH₃, R³═CH₂CH═CH₂

8g: R¹═CO₂t-Bu, R²═CH₂Ph, R³═CH₂CH═CH₂

13: R¹═CO₂Me, R²═H, R³═CH₂CH═CH₂

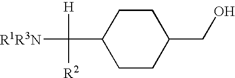

9a: R¹═CO₂t-Bu, R²═Me, R³═H

9b: R¹═CO₂t-Bu, R₂═CH₂CH₂CH₃, R³═H

9c: R¹═CO₂t-Bu, R²═CH₂CH═CH₂, R³═CH₃

9d: R¹═CO₂Me, R²═CH₂CH═CH₂, R₃═H

9e: R¹═CO₂t-Bu, R²═CH₂CH═CH₂, R³═CH₂Ph

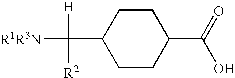

10a: R¹═CO₂t-Bu, R²═Me, R³═H

10b: R¹═CO₂t-Bu, R²═n-Pr, R³═H

10c: R¹═CO₂t-Bu, R²═CH₂CH═CH₂, R³═CH₃

10d: R¹═CO₂Me, R²═CH₂CH═CH₂, R³═H

10e: R¹═CO₂t-Bu, R²═CH₂CH═CH₂, R³═CH₂Ph

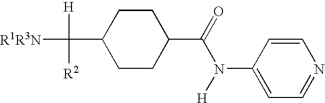

11a: R¹═CO₂t-Bu, R²═Me, R³═H

11b: R₁═CO₂t-Bu, R₂═n-Pr, R₃═H

11c: R¹═CO₂t-Bu, R²═CH₂CH═CH₂, R³═CH₃

11d: R¹═CO₂Me, R²═CH₂CH═CH₂, R³H

11e: R¹═CO₂t-Bu, R²═CH₂CH═CH₂, R³═CH₂Ph

12a: R¹, R³═H, R²═Me

12b: R¹, R³═H, R²═CH₂CH₂CH₃

12c: R¹═H, R²═CH₂CH═CH₂, R³═CH₃

12d: R¹, R³═H, R²═CH₂CH═CH₂

12e: R¹═H, R²═CH₂CH═CH₂, R³═CH₂Ph

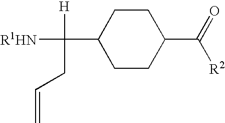

| | | |
|---|---|---|
| 14a: R¹ = CO₂Me, | R² = | 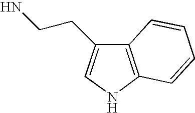 |
| 14b: R¹ = CO₂Me, | R² = | 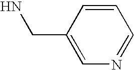 |
| 14c: R¹ = CO₂Me, | R² = | 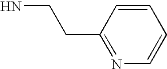 |
| 14d: R¹ = CO₂Me, | R² = | 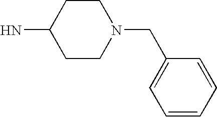 |
| 14e: R¹ = CO₂Me, | R² = | 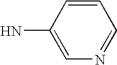 |
| 14f: R¹ = CO₂Me, | R² = | 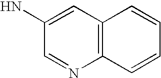 |
| 14g: R¹ = CO₂Me, | R² = | 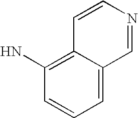 |
| 14h: R¹ = CO₂Me, | R² = | 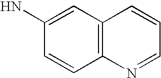 |
| 14i: R¹ = CO₂Me, | R² = | 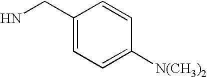 |
| 14j: R¹ = CO₂Me, | R² = | 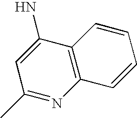 |
| 14k: R¹ = CO₂Me, | R² = | 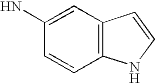 |
| 14l: R¹ = CO₂Me, | R² = | 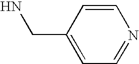 |
| 14m: R¹ = CO₂Me, | R² = | 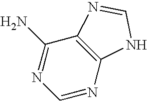 |
| 15a: R¹ = H, | R² = | 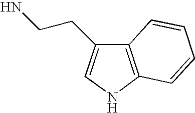 |
| 15b: R¹ = H, | R² = | 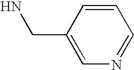 |
| 15c: R¹ = H, | R² = | 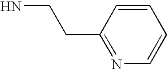 |
| 15d: R¹ = H, | R² = | 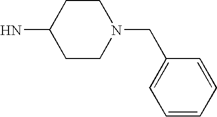 |
| 15e: R¹ = H, | R² = | 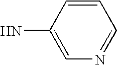 |
| 15f: R¹ = H, | R² = | 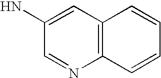 |
| 15g: R¹ = H, | R² = | 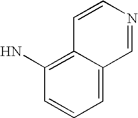 |
| 15h: R¹ = H, | R² = | 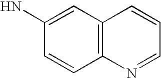 |
| 15i: R¹ = H, | R² = | 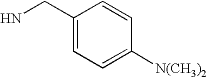 |
| 15j: R¹ = H, | R² = | 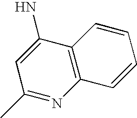 |
| 15k: R¹ = H, | R² = | 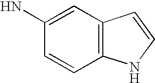 |
| 15l: R¹ = H, | R² = | 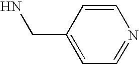 |
| 15m: R¹ = H, | R² = | 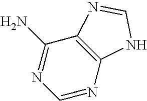 |

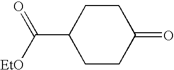

16

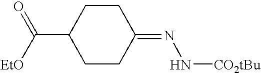

17

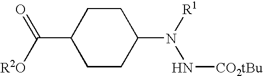

18: R₁═H, R²═CH₂CH₃

19a: R¹═CH₃, R²═CH₂CH₃

19b: R¹═CH₂CH₂CH₃, R²═CH₂CH₃

19c: R₁═CH₂CH₂CH(CH₃)₂, R²═CH₂CH₃

19d: R₁═CH(CH₃)₂, R²═CH₂CH₃

19e: R¹═CH₂Ph, R²═CH₂CH₃

19f: R¹═CH₂CH₂Ph, R²═CH₂CH₃

19g: R¹═CH₂CH(Ph)₂, R²═CH₂CH₃

19h: R¹═CH₂(C₆H₄)OCH₂Ph,

R₂═CH₂CH₃

19i:R¹═CH₂C₆H₁₁, R²═CH₂CH₃

19j:R¹═C₈H₁₇, R²═CH₂CH₃

19k: R¹═CH₂CHCHPh, R²═CH₂CH₃

20a: R₁═CH₃, R²═H

20b: R¹═CH₂CH₂CH₃, R²═H

20c: R¹═CH₂CH₂CH(CH₃)₂, R²═H

20d: R¹═CH(CH₃)₂, R²═H

20e: R¹═CH₂Ph, R²═H

20f: R¹CH₂CH₂Ph, R²═H

20g: R¹═CH₂CH(Ph)₂, R²═H

20h: R¹═CH₂(C₆H₄)OCH₂Ph,

R₂═H

20i: R¹═CH₂C₆H₁₁, R²═H

20j: R¹═C₈H₁₇, R²═H

20k: R¹═CH₂CHCHPh, R²═H

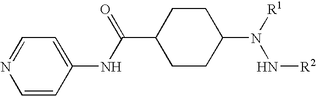

21a: R¹═CH₃, R²═CO₂tBu

21b: R¹═CH₂CH₂CH₃, R²═CO₂tBu

21c: R₁═CH₂CH₂CH(CH₃)2, R²═CO₂tBu

21d: R¹═CHC(CH₃)₂, R²═CO₂tBu

21e: R¹═CH₂Ph, R²═CO₂tBu

21f: R¹CH₂CH₂Ph, R²═CO₂tBu

21g: R¹═CH₂CH₂Ph), R²═CO₂tBu

21g: R¹═CH₂CH(Ph)₂, R²═CO₂tBu

21h: R¹═CH₂(C₆H₄)OCH₂Ph,

R₂═CO₂tBu

21i: R¹═CH₂C₆H₁₁, R²═CO₂tBu

21j: R¹═C₈H₁₇, R²═CO₂tBu

21k: R¹═CH₂CHCHPh, R²═CO₂tBu

22a: R¹═CH₃, R²═H

22b: R¹═CH₂CH₂CH₃, R²═H

22c: R¹═CH₂CH₂CH(CH₃)₂, R²═H

22d: R¹═CH(CH₃)₂, R²═H

22e: R¹═CH₂Ph, R₂═H

22f: R¹═CH₂CH₂Ph, R²═H

22g: R¹═CH₂CH(Ph)₂, R²═H

22h: R¹═CH₂(C₆H₄)OCH₂Ph,

R₂═H

22i: R¹═CH₂C₆H₁₁, R²═H

22j: R¹═C₈H₁₇, R²═H

22k: R¹═CH₂CHCHPh, R²═H

The following is a structural list which includes in more detail compounds of the present invention:

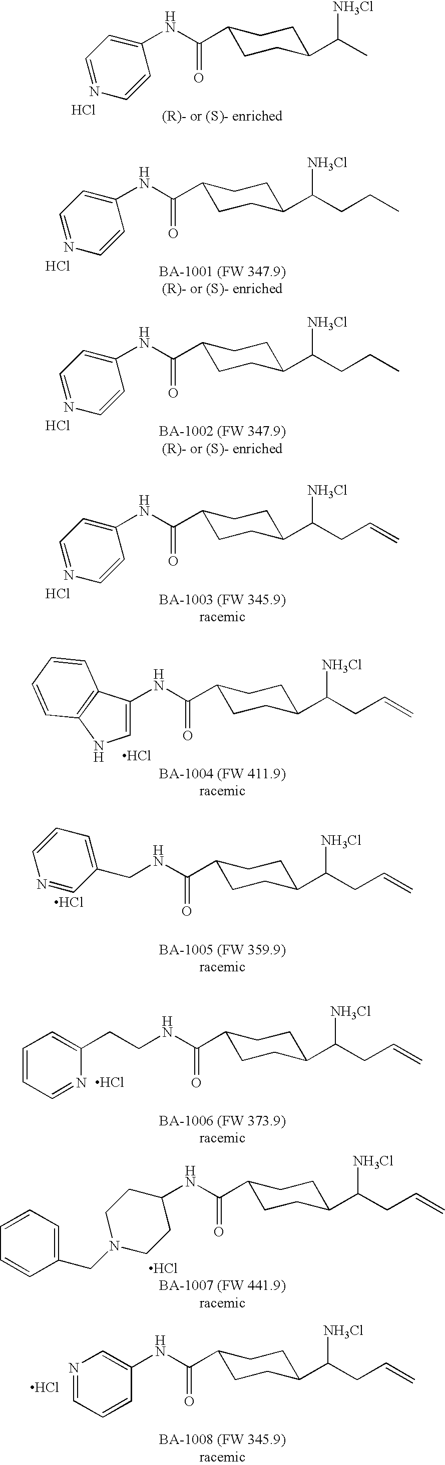

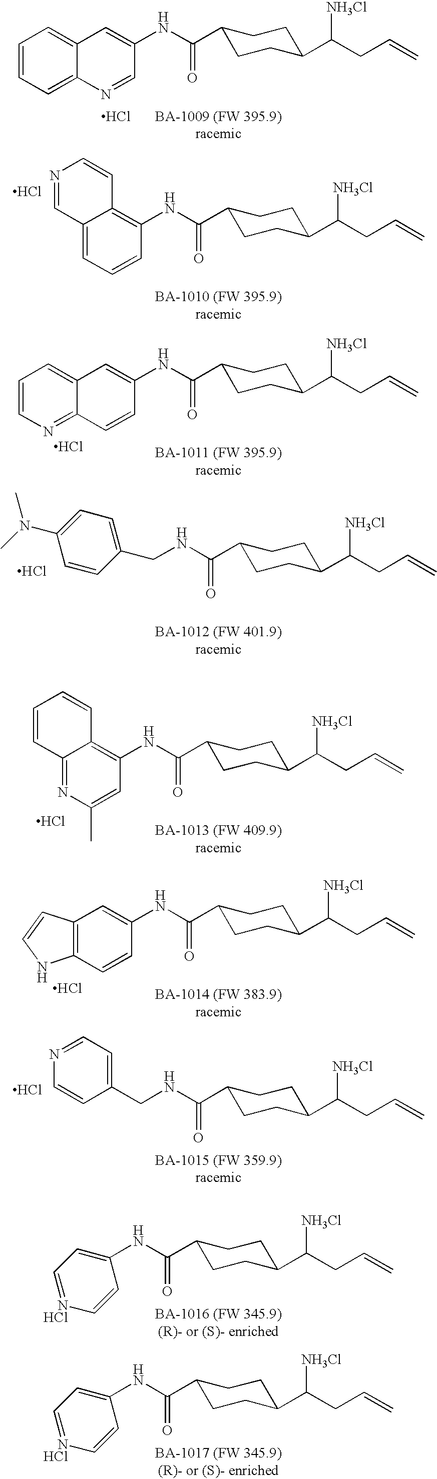

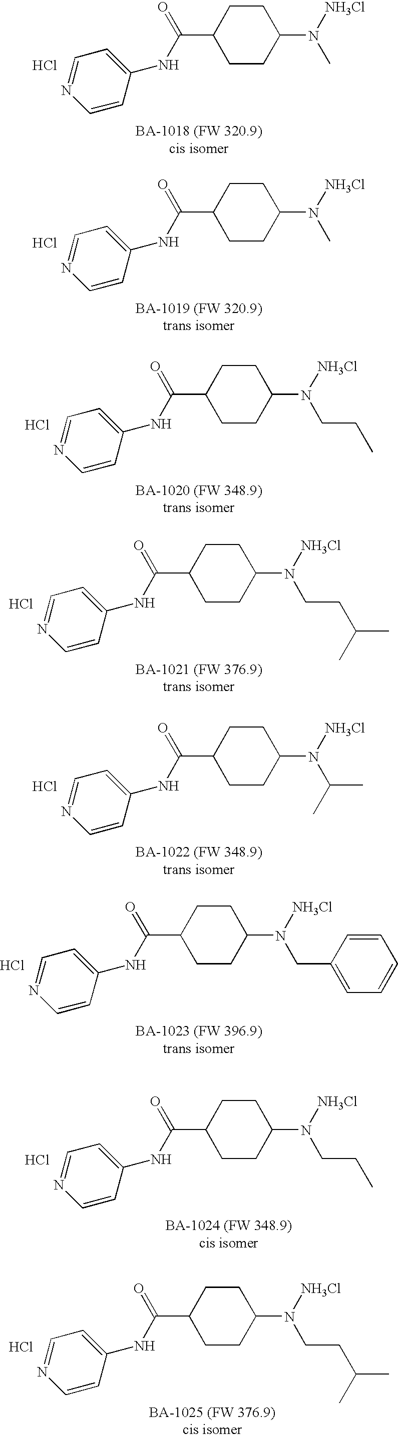

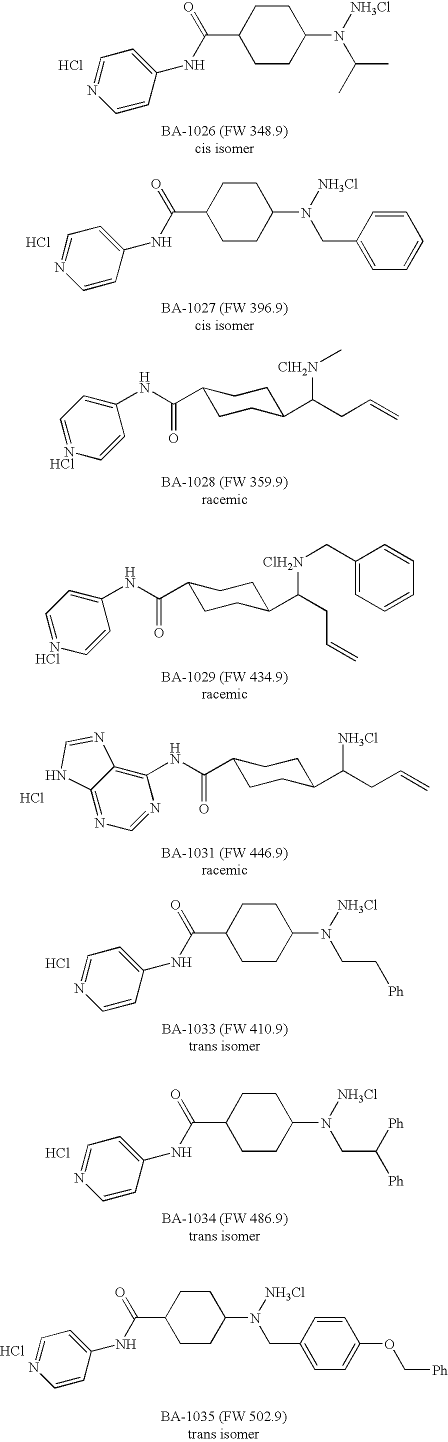

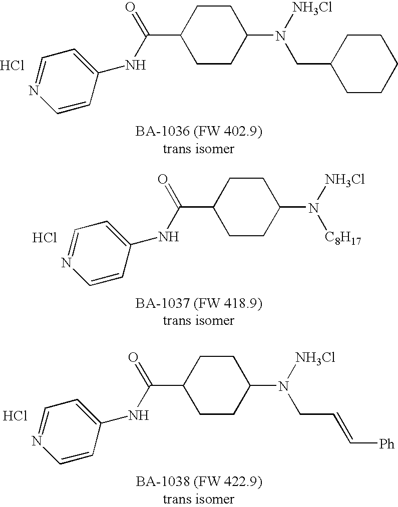

Previous syntheses of Y-27632[(R)-12a, Figure] and and other compounds have relied on the use of alpha-alkylbenzylamines as chiral educts which were subsequently acylated under Friedel-Crafts conditions at the para-position and reduced at the aromatic ring to provide the 1,4-substituted cyclohexane system (Arita et al., U.S. Pat. No. 5,478,838; Muro et al., U.S. Pat,. No. 4,997,834). One drawback to this approach has been the limited number of enantiomerically enriched alpha-alkylbenzylamines that are available commercially. A second significant problem that has restricted prior methods from synthesizing compounds possessing a wider range of molecular diversity has been the harsh conditions of the aromatic acylation and reduction chemistry which do not tolerate many functional groups such as unsaturated bonds (e.g. double bonds such as a double bond in an allyl group).

An alternative approach to the 1,4-substituted cyclohexane system has now been developed. The alternative method is specifically designed as a stereoselective means for generating compounds possessing a wide diversity of alkyl-branched amine moieties.

The alternative route begins with 1,4-cyclohexyldimethanol as an inexpensive starting material. Selective protection of one of the hydroxyl groups as silylether 2 followed by oxidation of the second unprotected alcohol to aldehyde 3 and reaction with an amine bearing a chiral auxiliary furnishes the corresponding imine 5 (4a when the chiral auxiliary is alpha-methylbenzyl, 4b when the imine is formed with benzylamine, 4c when the imine is formed with 1-amino-2-(methoxymethyl)pyrrolidine). Imine 5 is well suited for the synthesis of 1,4-substituted cyclohexane derivatives, because it may be reacted with a variety of nucleophilic organometallic reagents to diastereoselectively fumish different alkyl-branched amines 6 and 8. Stereocontrol is achieved in this synthesis by employing commercially available S- and R-chiral auxiliaries as directing groups for controlling the attack on imine 5 to provide the chiral alkyl-branched amino center, as well as resolving groups for separating diastereomeric secondary amines 6 and 8 produced from the nucleophilic addition to imine 5. In the synthesis of secondary amines by nucleophilic additions to imines, several systems featuring chiral auxiliaries have been employed: N-acylhydrazones (Friestad et al. J. Am. Chem. Soc. 2001, 123, 9922-9923 and refs therein), N-alkylhydrazones (Enders et al. Synlett. 1994, 795-797 and refs therein), aldoximes (Moody et al. Synlett. 1998, 733-734), alkylimines (Tanaka et al., Tetrahedron Lett. 1990, 31, 3023-3026; Yamamoto et al., J. Am. Chem. Soc. 1986, 108, 7778-7786; Alvaro et al., J. Chem. Soc., Perkin Trans. 1, 1996, 875-882). After the nucleophilic addition, the chiral auxiliary on 6 or 8 can be cleaved from the nitrogen using a variety of conditions such as hydrogenation, dissolving metal reductions, and hydride reductions to provide amino ethers 7 and 13. Effective reaction conditions and the diversity of nucleophiles that add with high diastereoselectivity to imines possessing chiral auxiliaries as well as the variety of methods for cleaving the auxiliary in the presence of sensitive functional groups, all make this approach an efficient, versatile means for synthesizing the target compounds.

The resulting amino ethers 6 and 8 are then converted to N-protected amino acid 10 by a route featuring removal of the auxiliary, protection of the amine as carbamate 8, silylether cleavage to alcohol 9 and oxidation of the primary alcohol to carboxylic acid 10. Amino acid 10 provides a second opportunity for generating a diverse series of analogs by modification of the amide moiety.

Several coupling strategies may be used to synthesize amides possessing a variety of different alkyl, cyclic-alkyl, aromatic and heteroaromatic substituents. For example, in the synthesis of BA-1001 and BA-1002[(R) and (S)-12b], symmetrical 1,4-cyclohexyldimethanol was used as a mixture of trans-:cis-diastereomers (˜3:1) and epimerization was achieved after the oxidation of the alcohol groups in order to enhance the desired thermodynamically stable trans-isomer. Selective protection of one of the two hydroxyl functions was achieved by treating 260 mol % of 1,4-cyclohexyldimethanol with 100 mol % of tert-butyldimethylsilylchloride in DMF at room temperature which provides about a 60% yield of the corresponding silylether 2. Oxidation of alcohol 2 was accomplished using PCC in CH₂Cl₂ at room temperature to provide aldehyde 3 in about 80% yield.

Imine 4a was next prepared quantitatively by condensing aldehyde 3 with α-methylbenzylamine in dichloromethane in the presence of MgSO₄ and isolated as a 8.5:1 trans-:cis-isomeric mixture as determined by integration of the methylidene protons at 7.58 and 7.74 ppm in the ¹H NMR spectrum. Diastereoselective additions of organometallic reagents to N-α-methylbenzyl aldimines can be used to prepare a variety of amines with good stereoselectivity. The addition of various organometallic reagents to imine 4a can provide a variety of different alkyl amines which can permit examination of the importance of stereochemistry and alkyl branched substituents. For example, allyl magnesium chloride (200 mol %) was added to a solution of copper iodide in dry THF at −30° C., then added dropwise to imine 4a in THF to afford the desired amine 6b in about 77% yield.

Conversion of amino ether 6b into its corresponding amino acid 10b was performed by removal of the α-methylbenzyl group from the amine by hydrogenation using a catalytic amount of Pd/C and ammonium formate in methanol at reflux for two hours. The primary amine 7b was obtained quantitatively and protected without further purification by treatment with (BOC)₂O in a mixed solvent system of NaHCO₃ and Na₂CO₃ dissolved in DME:H₂O to provide carbamate 8b in about 71% yield. Silylether 8b was cleaved with TBAF in THF at room temperature within 4 hours to provide alcohol 9b as a white solid in about 96% yield after chromatography. The desired acid 10b was synthesized by oxidation of alcohol 9b with TEMPO and a mixture of aqueous solutions of sodium chlorite and hypochlorite in about 85% yield.

The final amino amides BA-1001 and BA-1002[(R) and (S)-12b], were prepared from their respective acids (R) and (S)-10b. Initially, the carboxylic acid was coupled to 4-aminopyridine using TBTU as activating agent which provided the trans-diastereomer 11b. Subsequent removal of the BOC protecting group was effected in dry CH₂Cl₂ by bubbling of HCl gas. Removal of the solvent gave 12 as the HCl salt which was purified by trituration.

The enantiomeric purity of BA-1001 and BA-1002[(R) and (S)-12b] have been evaluated by coupling the chiral N-(p-toluenesulphonyl)-L-prolyl chloride using TEA followed by direct examination of the resulting amides by proton NMR. Two caracteristic triplets of the alkyl chains showed ratios of 9:1 in both cases indicating an enantiomeric excess of about 80% for the corresponding hydrochloride salts.

For another example, BA-1003[(R,S)-12d], was assembled effectively as a racemate by an approach featuring addition of allyl Grignard reagent to imine 4b, which was prepared from aldehyde 3 and benzylamine using similar conditions as described for 4a. Removal of the benzyl group from the resulting secondary amine 6d involved acylation with methylchloroformate to provide N-benzyl carbamate 8d and treatment with sodium in liquid ammonia which afforded carbamate 13 as well as its alcohol counterpart 9d from loss of the silyl ether. Silyl ether 13 was cleaved with TBAF, as described above for 8b, to afford in about 88% yield alcohol 9d. Acid 10d was then obtained in about 67% yield following the same conditions as for 10b. Carboxylic acid 10d was coupled to 4-aminopyridine using TBTU as activating agent to provide the trans-diastereomer 11d in about 86% yield. Finally, amino amide 12d (BA-1003) was isolated as its dihydrochloride salt after removal of the methylcarbamate with trimethylsilyliodide and treatment of the resulting amine with HCl gas in iso-propanol.

In order to explore the importance of the amide moiety on the biological activity, we prepared different analogs of BA-1003 possessing different aromatic rings. Following the same protocol as for the synthesis of BA-1003, the amines have been effectively coupled to the carboxylic acid 10d then deprotected to provide the corresponding dihydrochloric salts BA-1004 to BA-1015 and BA-1031[(R,S)-15a to (R,S)-15m].

Enantiomerically enriched BA-1016 and BA-1017[(R) and (S)-12c] were then prepared to verify the importance of the chirality on the biological activity of BA-1003. Allyl Grignard was added to the SAMP/RAMP-hydrazones 4c and the resulting anion was trapped by methyl chloroformate to afford the homoallylcarbamate 8e in about 79% yield. Removal of the chiral auxilliary was achieved by a reduction with lithium in liquid ammonia to provide the carbamate 13. The final hydrochloric salts BA-1016 and BA-1017[(R) and (S)-12c] were prepared from the corresponding carbamates 13 following the same protocole as described for BA-1003. The enantiomeric purity of the two compounds have been evaluated using the same method as described for BA-1001 and BA-1002. An enantiomeric excess of about 80% was measured in both cases by examination of the caracteristic multiplets of the allyl chain.

Finally, the racemic N-alkylated analogs BA-1028 and BA-1029[(R,S)-12d and (R,S)-12e] were prepared effectively using a similar approach as for (R,S)-12c. In this synthesis, the original substituents on the starting imines were kept and the tert-butyl carbamate was used instead of the methyl carbamate. The N-methyl and the N-benzyl analogs were synthesized in order to verify the effect of substitution of the original amine on the biological activity.

A number of aza-analogues were also prepared possessing different alkylhydrazine moieties as well as both cis- and trans-isomer of the disubstituted cyclohexane.

The exploited approach is a linear approach to generate diversity. Using ethyl 4-hydroxycyclohexanecarboxylate as starting materiel, the cis and trans-isomer of 18 were effectively prepared as the starting scaffolds for the preparation of the library. By reductive amination with the corresponding aldehydes, the alkylhydrazines 19 were prepared in good to moderate yields. The final dihydrochloric salts BA-1018 to BA-1027 and BA-1033 to BA-1038 (22) were easily obtained by a sequence of hydrolysis with sodium hydroxide, amide-bound formation and deprotection using the same conditions as for the preparation of hydrochloric acid salts 12 and 15.

Experimental Section

General. Melting points are uncorrected. Acetone was distilled from potassium carbonate. Methylchloroformate and oxalyl chloride were purified by fractional distillation prior to use. Triethylamine (TEA) and diisopropylethylamine (DIEA) were distilled from ninhydrin and then from CaH₂. Tetrahydrofuran (THF) was distilled from sodium/benzophenone. Toluene was distilled from sodium. Dichloromethane (DCM) and chloroform were distilled from phosphorus pentoxide. Unless otherwise noted, all chemicals were purchased from Aldrich Chemicals Inc. and used without further purification. Column chromatography was performed on 230-400 Mesh silica gel. Proton nuclear magnetic resonance (¹H NMR) spectra were recorded on Bruker ARX400 and AV400 spectrometers in deuterated chloroform (CDCl₃) and methanol (CD₃OD). Chemical shifts are reported in ppm (δ units) relative to residual solvent signals. Coupling constants (j) are reported in Hertz (Hz). Mass spectral data and HRMS (FAB and MAB) were obtained by the Universite de Montreal Mass Spectrometry facility.

### EXAMPLE 1

### Preparation of [(4′-tert-Butyldimethylsilyloxymethyl)-cyclohexyl]methanol (2)

To a stirred solution of 1,4-cyclohexyldimethanol (a mixture of cis/trans isomers, 30.0 g, 208 mmol) in DMF (200 mL) at room temperature, triethylamine (29.0 mL, 208 mmol) was added followed after 5 minutes by dimethylaminopyridine (1.10 g, 10.0 mmol) and tert-butyldimethylsilylchloride (12.0 g, 79.6 mmol). The mixture was stirred overnight at room temperature, quenched with distilled water (100 mL) and evaporated under reduced pressure using a rotary evaporator. The resulting syrup was partitioned between ethyl acetate (EtOAc, 250 mL) and saturated aqueous ammonium chloride (NH₄Cl sat., 75 mL). The two phases were separated and the aqueous phase was extracted with EtOAc (2×50 mL). The combined organic phases were washed with water (2×50 mL) and brine (1×50 mL), dried over sodium sulfate (Na₂SO₄) and evaporated to a residue that was purified by column chromatography using a gradient of 15 to 30% of EtOAc in hexane as eluant to give 12.5 g (61%) of the silyl ether 2 as a clear oil: HRMS calcd for C₁₄H₃₀O₂Si (M+): 258.2015, found: 258.2017; ¹H NMR (CDCl₃) showed a 70:30 ratio of isomers as measured by the isomeric signals at 3.14 and 3.55 ppm. Signals for the major isomer are as follows: δ 0.04 (s, 6H), 0.89 (s, 9H), 0.95 (m, 2H), 1.34-1.60 (m, 5H), 1.82 (m, 3H), 3.41 (d, 2H, J=6.35), 3.45 (d, 2H, J=6.34). Distinct signals for the minor isomer include: δ 3.48 (d, 2H, J=6.90), 3.55 (d, 2H, J=7.04).

### EXAMPLE 2

### Preparation of N-1(4′-tert-Butyldimethylsilyloxymethyl-cyclohexyl)methylidene]-1-phenylethanamine (4a)

A suspension of [4′-(tert-butyldimethylsilyloxy-methyl)cyclohexyl]methanol (2, 5.20 g, 20.2 mmol) and Celite™ (10 g) in dry DCM (200 mL) was treated with pyridinium chlorochromate (8.60 g, 39.9 mmol), stirred at room temperature for 3 hours, and filtered on Celite™. The filtrate was evaporated to a dark residue that was purified by column chromatography using 10% EtOAc in hexane as eluant to give 4.13 g (80%) of aldehyde 3 as a clear oil which was immediately used in the next step.

A solution of aldehyde 3 from above in dry DCM (50 mL/g) was treated with either (R)- or (S)-2-phenylethanamine (100 mol %) followed by magnesium sulfate (10 g/g of aldehyde), stirred overnight at room temperature and filtered. The filtrate was evaporated to dryness to give quantitatively the pure imine 4a as a low melting solid.

(R)-N-[(4′-tert-Butyldimethylsilyloxymethylcyclohexyl)methylidene]-1-phenylethanamine [(R)-4a]: ¹H NMR (CDCl₃) showed a 85:15 ratio of isomers as measured by the isomeric signals at 7.58 and 7.74 ppm. Signals for the major isomer are as follows: δ 0.04 (s, 6H), 0.89 (s, 9H), 0.95 (m, 2H), 1.27 (m, 2H), 1.40-1.67 (m, 5H), 1.88 (m, 3H), 2.20 (m, 1H), 3.41 (d, 2H, J=6.30), 4.25 (m, 1H), 7.20-7.40 (m, 5H), 7.58 (d, 1H, J=5.66). Distinct signals for the minor isomer include: 6 7.74 (d, 1H, J=4.20).

(S)-N-[(4′-tert-Butyldimethylsilyloxymethylcyclohexyl)methylidene]-1-phenylethanamine [(S)-4a]: m/z (FAB) 359.9 [(MH)⁺].

### EXAMPLE 3

### Preparation of N-[4′-(tert-Butyldimethylsilyloxymethyl-cyclohexyl)methylidene] benzylamine (4b)

A solution of aldehyde 3 (1.62 g, 6.30 mmol) prepared as described above in dry DCM (100 mL) was treated with benzylamine (0.71 mL, 6.50 mmol) followed by magnesium sulfate (20 g), stirred overnight at room temperature and filtered. The filtrate was evaporated to dryness to give quantitatively the pure imine 4b as a low melting solid: m/z (FAB) 346.2 [(MH)⁺]; ¹H NMR (CDCl₃) showed a 82:18 ratio of isomers as measured by the isomeric signals at 7.65 and 7.79 ppm. Signals for the major isomer are as follows: δ 0.05 (s, 6H), 0.91 (s, 9H), 0.98 (m, 2H), 1.28 (m, 2H), 1.46 (m, 1H), 1.62 (m, 1H), 1.89 (m, 3H), 2.20 (m, 1H), 3.43 (d, 2H, J=6.25), 4.56 (s, 2H), 7.20-7.38 (m, 5H), 7.65 (d, 1H, J=5.07). Distinct signals for the minor isomer include: δ 4.61 (s, 2H), 7.79 (s, 1H).

### EXAMPLE 4

### Preparation of N-[4′-(tert-Butyldimethylsilyloxymethyl-cyclohexyl)methylidene]-1-amino-2-(methoxymethyl)pyrrolidine (4c)

A solution of aldehyde 3 prepared as described above in dry DCM (50 mL/g) was treated with either (R)- or (S)-1-amino-2-(methoxymethyl)pyrrolidine (100 mol %) followed by magnesium sulfate (10 g/g of aldehyde), stirred overnight at room temperature and filtered. The filtrate was evaporated to dryness to give quantitatively the pure imine 4c as an oil.

(S)-N-[4′-(tert-Butyldimethylsilyloxymethylcyclohexyl)methylidene]-1-amino-2-(methoxymethyl)pyrrolidine [(S)-4c]: HRMS calcd for C₂₀H₄₀N₂O₂Si (M⁺): 368.2859, found: 368.2848; )⁺]; ¹H NMR (CDCl₃) showed a 7:3 ratio of isomers as measured by the isomeric signals at 6.49 and 6.63 ppm. Signals for the major isomer are as follows: δ 0.01 (s, 6H), 0.87 (s, 9H), 0.90-1.95 (m, 13H), 2.08 (m, 1H), 1.68 (m, 1H), 3.27-3.45 (m, 8H), 3.55 (m, 1 H), 6.49 (d, 1 H, J=6.01). Distinct signals for the minor isomer include: δ2.39 (m, 1H), 6.63 (d, 1H, J=5.14).

### EXAMPLE 5

### Preparation of N-[4′-(tert-Butyldimethylsilyloxymethyl-cyclohexyl)methylidene] methylamine (4d)

A solution of aldehyde 3 (1.48 g, 5.78 mmol) prepared as described above, in dry THF (80 mL), was treated with methylamine in THF (2 M, 5.00 mL, 10.0 mmol) followed by magnesium sulfate (15 g), stirred overnight at room temperature and filtered. The filtrate was evaporated to dryness to give quantitatively the pure imine 4d as an oil and was used immediately in the next step below (i.e. example 13) without any.

### EXAMPLE 6

### Preparation of (1R,1′R)-N[1′-(Phenyl)ethyl)]-1-[4″-(tert-Butyldimethylsilyloxymethyl)cyclohexyl]but-3-en-1-amine [(1R,1′R)-6b]

Allyl magnesium chloride in THF (2 M, 45.0 mL, 90.0 mmol) was added dropwise to a stirred suspension of copper iodide (8.57 g, 45.0 mmol) in dry THF (190 mL) at 40° C. The mixture was stirred for 15 min, cooled to −60° C., and treated dropwise with a solution of (R)-N-[(4′-tert-butyldimethylsilyloxymethylcyclohexyl)methylidene]-1-phenylethanamine (4a, 3.23 g, 9.0 mmol) in THF (40 mL). The reaction was stirred at −60° C. for 2 hours then quenched with a solution of NH₄Cl sat. and 28% aqueous ammonia (1:1, 70 mL). The mixture was allowed to reach room temperature then was partitioned between EtOAc (200 mL) and water (60 mL). The two phases were separated and the aqueous phase was extracted with EtOAc (2×50 mL). The combined organic phases were washed with brine (1×50 mL), dried over Na₂SO₄ and evaporated to a residue that was purified by column chromatography using a gradient of 2 to 10% EtOAc in hexane as eluant to give 2.79 g (77%) of the corresponding amine 6b as an oil: m/z (FAB) 402.2 [(MH)⁺]; ¹H NMR (CDCl₃) showed a 75:25 ratio of isomers as measured by the isomeric signals at 3.38 and 3.42 ppm. Signals for the major isomer are as follows: δ 0.04 (s, 6H), 0.74-1.55 (m, 19H), 1.63 (m, 1H), 1.80 (m, 2H), 1.93 (m, 1H), 2.18-2.40 (m, 3H), 3.38 (d, 2H, J=6.39), 3.92 (m, 1H), 5.10 (m, 2H), 5.80 (m, 1H), 7.20-7.40 (m, 5H). Distinct signals for the minor isomer include: δ 0.06 (s, 6H), 3.42 (d, 2H, J=7.02), 5.62 (m, 1H).

### EXAMPLE 7

### Preparation of (1S,1′S)-N-[1′-(Phenyl)ethyl)]-1-[4′-(tert-butyldimethylsilyloxymethyl)cyclohexyl]but-3-en-1-amine [(1S,1′S)-6b]

Following the procedure described above, (s)-N-[(4′-tert-butyldimethylsilyloxymethylcyclohexyl)methylidene]-1-phenylethanamine (4a, 2.43 g, 6.77 mmol) was reacted to give 2.04 g (75%) of the corresponding amine 6b as an oil.

### EXAMPLE 8

### Preparation of (1R,1′R)-N-[1′-(Phenyl)ethyl)]-1-[4′-(tert-butyldimethylsilyloxymethyl)cyclohexyl]ethan-1-amine [(1R,1′R)-6a]

Methyl magnesium bromide in diethyl ether (3M, 8.8 mL, 26.4 mmol) was added dropwise to a stirred suspension of copper iodide (2.51 g, 13.2 mmol) in dry THF (60 mL) at −30° C. The mixture was stirred for 10 minutes at −30° C., cooled to −65° C. and treated with boron trifluoride diethyl etherate complex (1.67 mL, 13.2 mmol). The mixture was stirred for 5 minutes and treated dropwise with a solution of (R)-N-[(4′-tert-butyldimethylsilyloxymethylcyclohexyl)methylidene]-1-phenylethanamine (4a, 950 mg, 2.65 mmol) in dry THF (25 mL). The temperature was allowed to raise to −30° C. over 45 minutes and the mixture was stirred at −30° C. for an additional 3 hours. The reaction was then quenched with a solution of NH₄Cl sat. and 28% aqueous ammonia (1:1, 40 mL). After stirring at room temperature for 20 minutes, the phases were separated and the aqueous phase was extracted with EtOAc (2×60 mL). The combined organic layers were washed with brine (1×50 mL), dried over Na₂SO₄ and evaporated to a residue that was purified by column chromatography using 20% iso-propanol in chloroform as eluant to give 710 mg (71%) of the corresponding amine 6a as an oil: HRMS calcd for C₂₃H₄₁NOSi [(MH)⁺]: 375.2957, found: 375.2961; ¹H NMR (CDCl₃) showed a 85:15 ratio of isomers as measured by the isomeric signals at 3.40 and 3.54 ppm. Signals for the major isomer are as follows: δ 0.04 (s, 6H), 0.85-1.90 (m, 26H), 2.45 (m, 2H), 3.40 (d, 2H, J=6.35), 7.25-7.40 (m, 5H). Distinct signals for the minor isomer include: δ 3.54 (d, 2H. J=6.35 Hz).

### EXAMPLE 9

### Preparation of (1S,1′S)-N-[1′-(Phenyl)ethyl)]-1-[4′-(tert-butyldimethylsilyloxymethyl)cyclohexyl]ethanamine [(1S,1′S)-6a]

Following the procedure described above (example 8), (S)-N-[(4′-tert-butyldimethylsilyloxymethylcyclohexyl)methylidene]-1-phenylethanamine (4a, 2.02 g, 5.63 mmol) was reacted to give 1.70 g (81%) of the corresponding amine 6a as an oil.

### EXAMPLE 10

### Preparation of (R,S)-N-Benzyl-1-[4′-(tert-butyldimethylsilyloxymethyl)cyclohexyl]but-3-en-1-amine [(R,S)-6d].

Allyl magnesium chloride in THF (2 M, 12.7 mL, 25.4 mmol) was added dropwise to a stirred suspension of copper iodide (2.41 g, 12.65 mmol) in dry THF (50 mL) at −40° C. The mixture was stirred for 15 min, cooled to −60° C., and treated dropwise with a solution of N-[(4′-tert-butyldimethylsilyloxymethyl-cyclohexyl)methylidene]benzylamine (4b, 1.10 g, 3.19 mmol) in THF (10 mL). The reaction was stirred at −60° C. for 2 hours then quenched with a solution of NH₄Cl sat. and 28% aqueous ammonia (1:1, 15 mL). The mixture was allowed to reach room temperature then was partitioned between EtOAc (75 mL) and water (15 mL). The two phases were separated and the aqueous phase was extracted with EtOAc (2×30 mL). The combined organic phases were washed with brine (1×30 mL), dried over Na₂SO₄ and evaporated to a residue that was purified by column chromatography using a gradient of 2 to 10% EtOAc in hexane as eluant to give 1.00 g (81%) of the corresponding amine 6d as an oil: m/z (FAB) 388.3 [(MH)⁺]; HRMS calcd for C₂₁H₃₆NOSi [(M-C₃H₅)⁺]: 346.2566, found: 346.2571; ¹H NMR (CDCl₃) showed a 87:13 ratio of isomers as measured by the isomeric signals at 3.43 and 3.56 ppm. Signals for the major isomer are as follows: δ 0.08 (s, 6H), 0.91-1.60 (m, 16H), 1.84 (m, 4H), 2.18 (m, 1H), 2.30 (m, 1H), 2.46 (m, 1H), 3.43 (d, 2H, J=6.34), 3.79 (d, 2H, J=2.14), 5.10 (m, 2H), 5.81 (m, 1H), 7.24-7.36 (m, 5H). Distinct signals for the minor isomer include: δ 3.55 (d, 2H, J=7.28).

### EXAMPLE 11

### Preparation of (1R,2′S)-N-Methyloxycarbonyl-N-[2′-(methoxymethyl)pyrrolidino]-1-[4″-(tert-butyldimethylsilyloxymethyl) cyclohexyl]but-3-en-1-amine [(1R,2′S)-8e]

A stirred solution of (S)-N-[4′-(tert-butyldimethylsilyloxymethylcyclohexyl)-methylidene]-1-amino-2-(methoxymethyl)pyrrolidine (4c, 1.13 g, 3.07 mmol) in dry toluene (100 mL) was cooled at −78° C. then treated over 20 minutes with allyl magnesium chloride in THF (2M, 6.14 mL, 12.28 mmol). The reaction was stirred at −78° C. for 20 minutes then methylchloroformate (958μL, 12.4 mmol) was added. The mixture was stirred overnight at room temperature then was partitioned between diethyl ether (100 mL) and water (30 mL). The two phases were separated and the aqueous phase was extracted with diethyl ether (2×20 mL). The combined organic phases were washed with brine (1×30 mL), dried over Na₂SO₄ and evaporated to a residue that was purified by column chromatography using a gradient of 2 to 10% EtOAc in hexane as eluant to give 535 mg (37%) of the corresponding carbamate 8e as an oil: HRMS calcd for C₂₅H₄₉N₂O₄Si [(MH)⁺]: 469.3462, found:469.3448.

### EXAMPLE 12

### Preparation of (1S,2′R)-N-Methyloxycarbonyl-N-[2′-(methoxymethyl)pyrrolidino]-1-[4″-(tert-butyldimethylsilyloxymethyl)-cyclohexyl]but-3-en-1-amine [(1S,2′R)-8e]

Following the procedure described above (Example 11), (R)-N-[4′-(tert-butyldimethylsilyloxymethylcyclohexyl)methylidene]-1-amino-2-(methoxymethyl)-pyrrolidine (4c, 1.10 g, 2.99 mmol) was reacted to give 500 mg (36%) of the corresponding free carbamate 8e as an oil.

### EXAMPLE 13

### Preparation of (R,S)-N-tert-Butyloxycarbonyl-N-methyl-1-[4′-(tert-butyldimethylsilyloxymethyl)cyclohexyl]but-3-en-1-amine 1(R,S)-8f]

A solution of allyl magnesium chloride in THF (2 M, 2.60 mL, 5.20 mmol) in THF (50 mL) was cooled to −78° C. then treated with a solution of N-[4′-(tert-butyldimethylsilyloxymethylcyclohexyl)methylidene] methylamine (4d, 478 mg, 1.75 mmol) in dry THF (5 mL) over 10 min. The reaction was stirred at −78° C. for 2 hours then di-tert-butyldicarbonate (2.30 g, 10.5 mmol) was added. The mixture was stirred overnight at room temperature then was partitioned between diethyl ether (100 mL) and water (30 mL). The two phases were separated and the aqueous phase was extracted with diethyl ether (2×20 mL). The combined organic phases were washed with brine (1×30 mL), dried over Na₂SO₄ and evaporated to a residue that was purified by column chromatography using a gradient of 1 to 15% EtOAc in hexane as eluant to give 678 mg (94%) of the corresponding carbamate 8f as an oil: HRMS calcd for C₂₃H₄₆N₁O₃Si [(MH)⁺]: 412.3247, found: 412.3233; ¹H NMR (CDCl₃) showed a mixture of isomers and rotamers. Characteristic signals are as follows: δ -0.02 (m, 6H), 0.75-1.03 (m, 12H), 1.20-1.85 (m, 17H), 2.05 (m, 1H), 2.33 (m, 1H), 2.57-2.63 (m, 3H), 3.33-3.45 (m, 2H), 4.90-5.10 (m, 2H), 5.64 (m, 1H).

### EXAMPLE 14

### Preparation of (R,S)-N-tert-Butyloxycarbonyl-N-benzyl-1-[4′-(tert-butyldimethylsilyloxymethyl)cyclohexyl]but-3-en-1-amine 1(R,S)-8g]

Following the procedure described above, N-[4′-(tert-butyldimethylsilyloxymethylcyclohexyl)methylidene] benzylamine (4b, 1.25 g, 3.63 mmol) was reacted to give 717 mg (40%) of the corresponding carbamate 8g as an oil: HRMS calcd for C₂₉H₅₀N₁O₃Si [(MH)⁺]: 488.3560, found: 488.3575; ¹H NMR (CDCl₃) showed a mixture of isomers and rotamers. Characteristic signals are as follows: δ 0.03 (m, 6H), 0.60-1.00 (m, 12H), 1.27-1.85 (m, 17H), 2.20-2.43 (m, 2H), 3.30-3.50 (m, 2H), 4.17-4.40 (m, 2H), 3.85-5.00 (m, 2H), 5.47-5.75 (m, 1H), 7.15-7.35 (m, 5H).

### EXAMPLE 15

### Preparation of (R,S)-N-Methyloxycarbonyl-N-benzyl-1-[4′-(tert-butyldimethylsilyloxymethyl)cyclohexyl]but-3-en-1-amine [(R,S)-8d]

A solution of (R,S)-N-benzyl-1-[4′-(tert-butyldimethylsilyloxymethyl)-cyclohexyl]but-3-en-1-amine (6d, 445 mg, 1.15 mmol] in dry acetone (13 mL), was treated with potassium carbonate (950 mg, 6.88 mmol) followed by methyl chloroformate (400 μL, 5.18 mmol), heated to a reflux, stirred for 5 h, and filtered. The filtrate was evaporated to a residue that was partitioned between EtOAc (20 mL) and NH₄Cl sat. (5 mL). The two phases were separated and the aqueous phase was extracted with EtOAc (2×5 mL). The combined organic phases were washed with brine (1×10 mL), dried over Na₂SO₄ and evaporated to a residue that was purified by column chromatography using an eluant of 10% EtOAc in hexane to give 304 mg (59%) of the carbamate 8d as an oil: ¹H NMR (CDCl₃) showed a 85:15 ratio of isomers as measured by the isomeric signals at 3.35 and 3.45 ppm. Signals for the major isomer are as follows: δ 0.02 (m, 6H), 0.50-1.55 (m, 16H), 1.64 (m, 2H), 1.80 (m, 2H), 2.17-2.43 (m, 2H), 3.35 (d, 2H, J=6.33), 3.71 and 3.73 (two s, 3H, rotamers), 4.25-4.50 (m, 2H), 4.89 (m, 2H), 5.57 (m, 1H), 7.20-7.40 (m, 5H). Distinct signals for the minor isomer include: δ 3.45 (d, 2H, J=7.03).

### EXAMPLE 16

### Preparation of (R)-1-[4′-(tert-Butyldimethylsilyloxymethyl)-cyclohexyl]butan-1-amine [(R)-7b]

To a solution of (1R,1′R)-N-[1′-(phenyl)ethyl)]-1-[4′-(tert-butyldimethylsilyloxymethyl)cyclohexyl]but-3-en-1-amine (6b, 300 mg, 0.748 mmol) in methanol (30 mL), ammonium formate (300 mg, 4.76 mmol) was added followed by Pd/C (10% wt, 40 mg) and the mixture was heated at a reflux for 2 hours. After cooling to room temperature, the reaction was filtered and water (10 mL) was added to the filtrate. The mixture was extracted two times with DCM (40 mL and 15 mL) and the combined organic phases were washed with brine, dried over Na₂SO₄ and evaporated to dryness to give quantitatively the corresponding free amine 7b as an oil: HRMS calcd for C₁₇H₃₈NOSi (MH⁺): 300.2722, found: 300.2730; ¹H NMR (CDCl₃) showed a 90:10 ratio of isomers as measured by the isomeric signals at 3.37 and 3.55 ppm. Signals for the major isomer are as follows: δ 0.02 (s, 6H), 0.85-1.04 (m, 13H), 1.15-1.92 (m, 13H), 3.04 (m, 1H), 3.37 (d, 2H, J=6.22), 8.20 (br s, 2H). Distinct signals for the minor isomer include: δ 0.04 (s, 6H), 3.12 (m, 1H), 3.55 (m, 2H), 8.55 (br s, 2H).

### EXAMPLE 17

### (S)-1-[4′-(tert-Butyldimethylsilyloxymethyl)-cyclohexyl]butan-1-amine [(S)-7b]

Following the procedure described above (example 16), (1S,l′S)-N-[1′-(phenyl)ethyl)]-1-[4′-(tert-butyldimethylsilyloxymethyl)cyclohexyl]but-3-en-1-amine (6b, 400 mg, 0.998 mmol) was reacted to give quantitatively the corresponding free amine 7b as an oil.

### EXAMPLE 18

### Preparation of (R)-1-[4′-(tert-Butyldimethylsilyloxymethyl)-cyclohexyl]ethan-1-amine [(R)-7a]

Following the procedure described above (example 16), (1 R,1′R)-N-[1′-(phenyl)ethyl)]-1-[4′-(tert-butyldimethylsilyloxymethyl)cyclohexyl]ethan-1-amine (6a, 300 mg, 0.800 mmol) was reacted to give 200 mg (93%) of the corresponding free amine 7a as an oil: HRMS calcd for C₁₅H₃₃NOSi [(MH)⁺]: 271.2331, found: 271.2340; ¹H NMR (CDCl₃) showed a 85:15 ratio of isomers as measured by the isomeric signals at 2.70 and 2.78 ppm. Signals for the major isomer are as follows: δ 0.04 (s, 6H), 0.89-1.88 (m, 22H), 2.70 (m, 1H), 3.40 (d, 2H, J=6.32). Distinct signals for the minor isomer include: 6 2.78 (m, 1H), 3.54 (d, 2H, J=6.35).

### EXAMPLE 19

### Preparation of (S)-1-[4′-(tert-Butyldimethylsilyloxymethyl)-cyclohexyl]ethan-1-amine [(S)-7a]

Following the procedure described above (example 16), (1S,1 ′S)-N-[1′-(phenyl)ethyl)]-1-[4′-(tert-butyldimethylsilyloxymethyl)cyclohexyl]ethan-1-amine (6a, 1.61 g, 4.29 mol) was reacted to give 1.05 g (90%) of the corresponding free amine 7a as an oil.

### EXAMPLE 20

### Preparation of (R,S)-N-Methyloxycarbonyl-1-[4′-(tert-butyldimethylsilyloxymethyl)cyclohexyl]but-3-en-1-amine [(R,S)-13]

Anhydrous liquid ammonia (12 mL) was added to a solution of (R,S)-N-methyloxycarbonyl-N-benzyl-1-[4′-(tert-butyldimethylsilyloxymethyl)-cyclohexyl]but-3-en-1-amine (8d, 250 mg, 0.562 mmol) in dry THF (2.5 mL) at −78° C. After treating the reaction mixture with freshly cut sodium (52 mg, 2.3 mmol), the cooling bath was removed, and the blue reaction mixture was stirred at reflux until the color faded (about 15 minutes). The reaction mixture was cooled to −78° C., treated with a second portion of sodium (26 mg, 1.1 mmol) when the blue color reappeared. The cool bath was removed and the reaction was stirred at reflux for 1 h. The reaction was quenched with solid ammonium chloride (500 mg), and the ammonia was allowed to evaporate. The residue was partitioned between DCM (20 mL) and water (5 mL). The two phases were separated and the aqueous phase was extracted with DCM (2×5 mL). The combined organic phases were washed with brine (1×10 mL), dried over Na₂SO₄ and evaporated to a residue that was purified by column chromatography using a gradient of 10% EtOAc in hexane to 100% EtOAc as eluant. The first product eluted was the silyl ether (R,S)-13 (91 mg, 46%) followed by the alcohol (R,S)-9d (32 mg, 24%), namely:

(R,S)-N-Methyloxycarbonyl-1-[4′-(tert-butyldimethylsilyloxymethyl)-cyclohexyl]but-3-en-1-amine [(R,S)-13]: HRMS calcd for C₁₆H₃₂NO₃Si [(M-C₃H₅)⁺]: 314.2151, found: 314.2150; ¹H NMR (CDCl₃) showed a 85:15 ratio of isomers as measured by the isomeric signals at 3.37 and 3.48 ppm. Signals for the major isomer are as follows: δ 0.01 (m, 6H), 0.78-1.13 (m, 11H), 1.21-1.48 (m, 4H), 1.78 (m, 4H), 2.13 (m, 1H), 2.27 (m, 1H), 3.37 (d, 2H, J=6.27), 3.50-3.66 (m, 4H), 4.55 (d, 1H, J=9.50), 5.05 (m, 2H), 5.74 (m, 1H). Distinct signals for the minor isomer include: δ 3.48 (d, 2H, J=7.20);

(R,S)-N-Methyloxycarbonyl-1-[4′-(hydroxymethyl)cyclohexyl]but-3-en-1-amine [(R,S)-9d]: HRMS calcd for C₁₃H₂₃NO₃ (M+): 241.1678, found: 241.1677; ¹H NMR (CDCl₃) showed a 85:15 ratio of isomers as measured by the isomeric signals at 4.60 and 4.48 ppm. Signals for the major isomer are as follows:δ 0.83-1.14 (m, 3H), 1.26-1.53 (m, 3H), 1.70-1.93 (m, 4H), 2.14 (m, 1H), 2.26(m, 1H), 3.41 (d, 2H, J=6.28), 3.53 (m, 1 H), 3.63 (s, 3H), 4.60 (d, 1H, J=9.50), 5.06 (m, 2H), 5.73 (m, 1H). Distinct signals for the minor isomer include: 6 4.48 (d, 1 H, J=8.38).

### EXAMPLE 21

### Preparation of (1R)-N-Methyloxycarbonyl-1-[4′-(tert-butyldimethylsilyloxymethyl)cyclohexyl]but-3-en-1-amine [(1 R)-13]

Anhydrous liquid ammonia (12 mL) was added to a solution of (1 R,2′S)-N-Methyloxycarbonyl-N-[2′-(methoxymethyl)pyrrolidino]-1-[4″-(tert-butyldimethylsilyloxymethyl)cyclohexyl]but-3-en-1-amine (8e, 500 mg, 1.07 mmol) in dry THF (6 mL) at −78° C. After treating the reaction mixture with freshly cut lithium (60 mg, 8.6 mmol), the cooling bath was removed, and the reaction mixture was stirred at reflux for 1 h. The reaction was quenched with solid ammonium chloride (1 g), and the ammonia was allowed to evaporate. The residue was partitioned between DCM (50 mL) and water (10 mL). The two phases were separated and the aqueous phase was extracted with DCM (2×10 mL). The combined organic phases were washed with brine (1×20 mL), dried over Na₂SO₄ and evaporated to a residue that was purified by column chromatography using a gradient of 5% EtOAc in hexane to 100% EtOAc as eluant to give 64 mg (17%) of the corresponding silyl ether 13 as an oil.

### EXAMPLE 22

### Preparation of (1S)-N-Methyloxycarbonyl-1-[4′-(tert-butyldimethylsilyloxymethyl)cyclohexyl]but-3-en-1-amine [(1S)-13]

Following the procedure described above (example 21), (1S,2′R)-N-methyloxycarbonyl-N-[2′-(methoxymethyl)pyrrolidino]-1-[4″-(tert-butyldimethylsilyloxymethyl)-cyclohexyl]but-3-en-1-amine (8e, 500 mg, 1.07 mmol) was reacted to give 90 mg (24%) of the corresponding silyl ether 13 as an oil.

### EXAMPLE 23

### Preparation of (R)-N-tert-Butyloxycarbonyl-1-[4′-(tert-butyldimethylsilyloxymethyl)cyclohexyl]butan-1-amine [(R)-8b]

To a stirred solution of (R)-1-[4′-(tert-butyldimethylsilyloxymethyl)-cyclohexyl]butan-1-amine (7b, 70 mg, 0.23 mmol) in a mixture of dimethoxyethane and water (1:1 v:v, 2 mL) was added sodium carbonate (25 mg, 0.24 mmol) and sodium bicarbonate (20 mg, 0.24 mg) followed by di-tert-butyl dicarbonate (56 mg, 0.26 mmol). The mixture was stirred at room temperature for 3 hours then was partitioned between NH₄Cl sat. (2 mL) and EtOAc (5 mL). The two phases were separated and the aqueous phase was extracted with EtOAc (2×3 mL). The combined organic phases were washed with brine (1×4 mL), dried over Na₂SO₄ and evaporated to a residue that was purified by column chromatography using an eluant of 5% EtOAc in hexane to give 68 mg (74%) of the corresponding carbamate 8b as an oil: ¹H NMR (CDCl₃) showed a 85:15 ratio of isomers as measured by the isomeric signals at 3.38 and 3.51 ppm. Signals for the major isomer are as follows: δ 0.03 (s, 6H), 0.81-1.13 (m, 15H), 1.20-1.50 (m, 16H), 1.65-1.86 (m, 4H), 3.38 (d, 2H, J=6.28), 3.44 (m, 1H), 4.27 (d, 1H, J=9.79). Distinct signals for the minor isomer include: δ 0.04 (s, 6H), 3.51 (d, 2H, J=7.14).

### EXAMPLE 24

### Preparation of (S)-N-tert-Butyloxycarbonyl-1-[4′-(tert-butyldimethylsilyloxymethyl)cyclohexyl]butan-1-amine [(S)-8b]

Following the procedure described above (example 23), (S)-1-[4′-(tert-butyldimethylsilyloxymethyl)cyclohexyl]butan-1-amine (7b, 297 mg, 0.993 mmol) was reacted to give 275 mg (69%) of the corresponding carbamate 8b as an oil: m/z (FAB) 400.2 [(MH)⁺].

### EXAMPLE 25

### Preparation of (R)-N-tert-Butyloxycarbonyl-1-[4′-(tert-butyldimethylsilyloxymethyl)cyclohexyl]ethan-1-amine [(R)-8a]

Following the procedure described above (example 23), (R)-1-[4′-(tert-butyldimethylsilyloxymethyl)cyclohexyl]ethan-1-amine (7a, 400 mg, 1.48 mmol) was reacted to give 514 mg (94%) of the corresponding carbamate 8a as an oil: HRMS calcd for C₂₀H₄₁NO₃Si (M⁺): 370.2777, found: 370.2770; ¹H NMR (CDCl₃) δ 0.03 (m, 6H), 0.89-1.84 (m, 31H), 3.39 (d, 2H, J=6.3), 3.48 (m, 1H), 4.39 (m, 1H).

### EXAMPLE 26

### Preparation of (S)-N-tert-Butyloxycarbonyl-1-[4′-(tert-butyldimethylsilyloxymethyl)cyclohexyl1ethan-1-amine [(S)-8a]

Following the procedure described above (example 23), (S)-1-[4′-(tert-butyldimethylsilyloxymethyl)cyclohexyl]ethan-1-amine (7a, 1.19 g, 4.38 mmol) was reacted to give 1.38 g (85%) of the corresponding carbamate 8a as an oil.

### EXAMPLE 27

### Preparation of (R)-N-tert-Butyloxycarbonyl-1-[4′-(hydroxymethyl)cyclohexyl]butan-1-amine [(R)-9b]

A stirred solution of (R)-N-tert-butyloxycarbonyl-1-[4′-(tert-butyldimethylsilyloxymethyl)cyclohexyl]butan-1-amine (8b, 50 mg, 0.13 mmol) in dry THF (2.5 mL) was cooled to 0° C., treated in one portion with a solution of tetrabutylammonium fluoride in THF (1.0 M, 0.50 mL, 0.50 mmol), stirred at room temperature for 3 hours, and partitioned between NH₄Cl sat. (2 mL) and EtOAc (5 mL). The two phases were separated and the aqueous phase was extracted with EtOAc (2×3 mL). The combined organic phases were washed with brine (1×4 mL), dried over Na₂SO₄ and evaporated to a residue that was purified by column chromatography using 100% EtOAc as eluant to give 34 mg (92%) of alcohol 9b as a white solid: HRMS calcd for C₁₆H₃₂NO₃ [(MH)⁺]: 286.2382, found: 286.2375; ¹H NMR (CDCl₃) showed a 90:10 ratio of isomers as measured by the isomeric signals at 3.45 and 3.59 ppm. Signals for the major isomer are as follows: δ 0.88-1.55 (m, 22H), 1.74-1.90 (m, 4H), 3.43-3.50 (m and d, 3H, J=6.30), 4.27 (d, 1 H, J=10.55). Distinct signals for the minor isomer include: δ 3.59 (d, 2H, J=7.31).

### EXAMPLE 28

### Preparation of (S)-N-tert-Butyloxycarbonyl-1-[4′-(hydroxymethyl)cyclohexyl]butan-1-amine [(S)-9b]

Following the procedure described above (example 27), (S)-N-tert-butyloxycarbonyl-1-[4′-(tert-butyldimethylsilyloxymethyl)cyclohexyl]butan-1-amine (8b, 241 mg, 0.604 mmol) was reacted to give 166 mg (96%) of the corresponding alcohol 9b as a white solid.

### EXAMPLE 29

### Prepartion of (R)-N-tert-Butyloxycarbonyl-1-[4′-(hydroxymethyl)cyclohexyl]ethan-1-amine [(R)-9a]

Following the procedure described above (example 27), (R)-N-tert-butyloxycarbonyl-1-[4′-(tert-butyldimethylsilyloxymethyl)cyclohexyl]ethan-1-amine (8a, 1.10 g, 2.97 mmol) was reacted to give 0.680 g of the corresponding alcohol 9a (89%) as a white solid: mp=104-106° C.; HRMS calcd for C₁₄H₂₇NO₃ (M⁺): 257.1990, found: 257.1994; ¹H NMR (CDCl₃) δ 0.85-1.90 (m, 22H), 3.45 (d, 2H, J=6.27), 3.55 (m, 1H), 4.40 (m, 1H).

### EXAMPLE 30

### Preparation of (S)-N-tert-Butyloxycarbonyl-1-[4′-(hydroxymethyl)cyclohexyl]ethan-1-amine [(S)-9a]

Following the procedure described above (example 27), (S)-N-tert-butyloxycarbonyl-1-(4′-tert-butyldimethylsilyloxymethylcyclohexyl)ethan-1-amine (8a, 705 mg, 1.91 mmol) was reacted to give 442 mg (90%) of the corresponding alcohol 9a as a white solid.

### EXAMPLE 31

### Preparation of (R,S)-N-Methyloxycarbonyl-1-[4′-(hydroxymethyl)cyclohexyl]but-3-en-1-amine [(R,S)-9d]

Following the procedure described above (example 27), (R,S)-N-methyloxycarbonyl-1-[4′-(tert butyldimethylsilyloxymethyl)cyclohexyl]but-3-en-1-amine (13, 90 mg, 0.25 mmol) was reacted to give 53 mg (88%) of the corresponding alcohol 9d as an oil.

### EXAMPLE 32

### Preparation of (R)-N-Methyloxycarbonyl-1-[4′-(hydroxymethyl)cyclohexyl]but-3-en-1-amine [(R)-9d]

Following the procedure described above (example 27), (R)-N-methyloxycarbonyl-1-[4′-(tert butyldimethylsilyloxymethyl)cyclohexyl]but-3-en-1-amine (13, 60 mg, 0.17 mmol) was reacted to give 33 mg (81%) of the corresponding alcohol 9d as a low melting solid.

### EXAMPLE 33

### Preparation of (S)-N-Methyloxycarbonyl-1-[4′-(hydroxymethyl)cyclohexyl]but-3-en-1-amine [(s)-9d]

Following the procedure described above (example 27), (S)-N-methyloxycarbonyl-1-[4′-(tert butyldimethylsilyloxymethyl)cyclohexyl]but-3-en-1-amine (13, 90 mg, 0.25 mmol) was reacted to give 52 mg (88%) of the corresponding alcohol 9d as a low melting solid.

### EXAMPLE 34

### Preparation of (R,S)-N-tert-Butyloxycarbonyl-N-methyl-1-[4′-(hydroxymethyl)cyclohexyl]but-3-en-1-amine [(R,S)-9c]

Following the procedure described above (example 27), (R,S)-N-tert-butyloxycarbonyl-N-methyl-1-[4′-(tert-butyldimethylsilyloxymethyl)cyclohexyl]but-3-en-1-amine [(R,S)-8f, 559 mg, 1.36 mmol] in dry THF (25 mL) was reacted to give 340 mg (84%) of the corresponding alcohol 9c as an oil: HRMS calcd for C₁₇H₃₂N₁O₃ [(MH)⁺]: 298.2382, found: 298.2389; ¹H NMR (CDCl₃) showed a mixture of isomers and rotamers. Characteristic signals are as follows: δ 0.73-1.00 (m, 3H), 1.17-1.80 (m, 17H), 1.98 (m, 1H), 2.30 (m, 1H), 2.47-2.58 (m, 3H), 3.27-3.43 (m, 2H), 4.83-4.98 (m, 2H), 5.58 (m, 1H).

### EXAMPLE 35

### Preparation of (R,S-N-tert-Butyloxycarbonyl-N-benzyl-1-[4′-(hydroxymethyl)cyclohexyl]but-3-en-1-amine 1(R,S)-9e]

Following the procedure described above (example 27), (R,S)-N-tert-butyloxycarbonyl-N-benzyl-1-[4′-(tert-butyldimethylsilyloxymethyl)cyclohexyl]but-3-en-1-amine [(R,S)-8g, 601 mg, 1.23 mmol] was reacted to give 403 mg (88%) of the corresponding alcohol 9e as an oil: HRMS calcd for C₂₃H₃₆N₁O₃ [(MH)⁺]: 374.2695, found: 374.2689.

### EXAMPLE 36

### Preparation of (R)-4-[N′-(tert-Butyloxycarbonyl)butan-1′-amino]cyclohexane Carboxylic Acid [(R)-10b]

To a stirred solution of (R)-N-tert-butyloxycarbonyl-1-[4′-(hydroxymethyl)-cyclohexyl]butan-1-amine (9b, 27 mg, 0.095 mmol) in acetonitrile (0.50 mL) at room temperature, phosphate buffer (pH 6.7, 0.35 mL) was added followed by 2,2,6,6-tetramethyl-1-piperidinyloxy free radical (TEMPO, 1.0 mg, 0.006 mmol). The solution was warmed to 35° C. then treated with an aqueous solution of sodium chlorite (9.14 g of 80% NaClO₂ in 40 mL of distilled water, 0.093 mL) followed after 2 minutes by an aqueous solution of sodium hypochlorite (0.53 mL of 10.8% NaClO in 20 mL distilled water, 0.047 mL). The two phase solution was stirred at 35° C. for 3 hours, treated with additional portions of the aqueous solutions of sodium chlorite (0.093 mL) and sodium hypochlorite (0.047 mL), stirred overnight at 35° C., and treated with EtOAc (4 mL). The phases were separated and the aqueous phase was extracted with EtOAc (2×1 mL). The combined organic phases were washed with brine (1×2 mL), dried over Na₂SO₄ and evaporated to dryness to give 21 mg (74%) of the carboxylic acid as a white solid. Carboxylic acid 10b was sufficiently pure to be use in the next step without further purification: mp=143-145° C.; HRMS calcd for C₁₆H₃₀NO₄ [(MH)⁺]: 300.2175, found: 300.2164; ¹H NMR (CDCl₃) δ 0.88-1.51 (m, 21H), 1.82 (m, 2H), 2.07 (m, 2H), 2.28 (m, 1H), 3.46 (m, 1H), 4.25 (d, 1H, J=9.93). ). Amount of the minor isomer is not measurable by proton NMR.

### EXAMPLE 37

### Preparation of (S)4-[N-(tert-Butyloxycarbonyl)butan-1′-amino]cyclohexane Carboxylic Acid [(S)-10b]

Following the procedure described above (example 36), (S)-N-tert-butyloxycarbonyl-1-[4′-(hydroxymethyl)cyclohexyl]butan-1-amine (9b, 165 mg, 0.579 mmol) was oxidized to give 148 mg (85%) of the corresponding carboxylic acid 10b as a white solid.

### EXAMPLE 38

### Preparation of (R,S)-4-[N′-(Methyloxycarbonyl)but-3′-en-1′-amino]cyclohexane Carboxylic Acid [(R,S)-10d]

Following the procedure described above (example 36), (R,S)-N-methyloxycarbonyl-1-[4′-(hydroxymethyl)cyclohexyl]but-3-en-1-amine (9d, 30 mg, 0.12 mmol) was oxidized to give 15 mg (67%) of the carboxylic acid 10d as a low melting solid. The carboxylic acid was sufficiently pure to be used in the next step without further purification: HRMS calcd for C₁₃H₂₁NO₄ (M⁺): 255.1471, found: 255.1474; ¹H NMR (CDCl₃) showed a 85:15 ratio of isomers as measured by the isomeric signals at 5.08 and 5.35 ppm. Signals for the major isomer are as follow: δ 0.96-1.50 (m, 5H), 1.85 (m, 2H), 2.00-2.37 (m, 5H), 3.40-3.73 (s+m, 4H), 4.50 (d, 1H, J=9.61), 5.08 (m, 2H), 5.74 (m, 1H). Distinct signal for the minor isomer include: δ 5.35 (br s, 1H).

### EXAMPLE 39

### Preparation of (R)-4-[N′-(Methyloxycarbonyl)but-3′-en-1′-amino]cyclohexane Carboxylic Acid [(R)-10d]

Following the procedure described above (example 36), (R)-N-methyloxycarbonyl-1-[4′-(hydroxymethyl)cyclohexyl]but-3-en-1-amine (9d, 30 mg, 0.12 mmol) was oxidized to give 21 mg (68%) of the corresponding carboxylic acid 10d as a white solid.

### EXAMPLE 40

### Preparation of (S)-4-[-N′-(Methyloxycarbonyl)but-3′-en-1′-amino] cyclohexane Carboxylic Acid [(S)-10d]

Following the procedure described above (example 36), (S)-N-methyloxycarbonyl-1-[4′-(hydroxymethyl)cyclohexyl]but-3-en-1-amine (9d, 51 mg, 0.21 mmol) was oxidized to give 34 mg (62%) of the corresponding carboxylic acid 10d as a white solid.

### EXAMPLE 41

### Preparation of (R,S)-4-[(N′-tert-Butyloxycarbonyl-N′-methyl)but-3′-en-1′-amino]cyclohexane Carboxylic Acid [(R,S)-10c]

Following the procedure described above (example 36), (R,S)-N-tert-butyloxycarbonyl-N-methyl-1-[4′-(hydroxymethyl)cyclohexyl]but-3-en-1-amine (9c, 320 mg, 1.08 mmol) was oxidized to give 336 mg (94%) of the corresponding carboxylic acid 10d as a white solid: HRMS calcd for C₁₇H₃₀N₁O₄ [(MH)⁺]: 312.2175, found: 312.2184; ¹H NMR (CDCl₃) showed a mixture of isomers and rotamers. Characteristic signals are as follows: δ 1.38-2.83 (m, 25H), 4.54 (m, 2H), 5.08 (m, 1H), 9.42 (brs, 1H).

### EXAMPLE 42

### Preparation of (R,S)-4-[(N′-tert-Butyloxycarbonyl-N′-benzyl)but-3′-en-1′-amino] cyclohexane Carboxylic Acid [(R,S)-10e]

Following the procedure described above (example 36), (R,S)-N-tert-butyloxycarbonyl-N-benzyl-1-[4′-(hydroxymethyl)cyclohexyl]but-3-en-1-amine (9e, 426 mg, 1.14 mmol) was oxidized to give 368 mg (83%) of the corresponding carboxylic acid 10e as a white solid: HRMS calcd for C₂₃H₃₄N₁O₄ [(MH)⁺]: 388.2488, found: 388.2478.

### EXAMPLE 43

### Preparation of (R)-trans-4[-N′-(tert-Butyloxycarbonyl)ethan-1′-amino]cyclohexane Carboxylic Acid [(R)-10a]

Freshly distilled oxalylchloride (0.500 mL, 5.73 mmol) was added to dry DCM (10 mL) at room temperature. The solution was cooled to 0° C., treated dropwise with anhydrous dimethylsulfoxide (0.826 mL, 11.6 mmol), stirred for 10 minutes, cooled to −50° C. then treated dropwise with a solution of (1R)-N-tert-butyloxycarbonyl-1-[4′-(hydroxymethyl)cyclohexyl]ethan-1-amine (9a, 750 mg, 2.92 mmol) in dry DCM (1 mL). The mixture was stirred for 15 minutes at −50° C., treated dropwise with freshly distilled triethylamine (2.84 mL, 20.37 mmol) and allowed to warm to 0° C. with stirring over 2 hours. The reaction was then quenched with NH₄Cl sat. (10 mL) and the two phases were separated. The aqueous phase was extracted with CH₂Cl₂ (2×10 mL). The combined organic phases were washed with brine (1×10 mL), dried over Na₂SO₄ and evaporated to a residue that was purified by column chromatography using a gradient of 0 to 50% of EtOAc in hexanes as eluant to give 655 mg (88%) of the corresponding aldehyde which was immediately used in the next step.

A solution of NaClO₂ (80%, 1.20 g, 10.61 mmol) and NaH₂PO₄ (1.6 g, 13.2 mmol) in water (12 mL) was added to a room temperature solution of the aldehyde (340 mg, 1.33 mmol,) in a mixture of tert-butanol and acetonitrile (1:1, 24 mL). After stirring for 30 minutes, the reaction mixture was partitioned between NH₄Cl sat (110 mL) and EtOAc (20 mL). The phases were separated and the aqueous phase was extracted with EtOAc (2×10 mL). The combined organic layers were washed with brine (10 mL), dried over Na₂SO₄ and evaporated to a residue that was purified by column chromatography using a gradient of 0 to 50% EtOAc in hexanes as eluant to give 300 mg (83%) of the corresponding carboxylic acid 10a as a white solid: mp=114-116° C.; HRMS calcd for C₁₄H₂₅NO₄ (M⁺): 271.1783, found: 271.1793; ¹H NMR (CDCl₃): δ 1.00-1.68 (m, 17H), 1.77-2.93 (m, 2H), 2.02-2.30 (m, 3H), 3.55 (m, 1H), 4.37 (m, 1H).

### EXAMPLE 44

### Preparation of (S)-trans-4-[N′-(tert-Butyloxycarbonyl)ethan-1′-amino] cyclohexane Carboxylic Acid [(S)-10a]

Following the procedure described above (example 43), (S)-N-tert-butyloxycarbonyl-1-[4′-(hydroxymethyl)cyclohexyl]ethan-1-amine (9a, 574 mg, 2.25 mmol) was oxidized in two steps to give 470 mg (77%) of the corresponding carboxylic acid 10a as a white solid.

### EXAMPLE 45

### General Procedure for Preparation of Amides of type 11 (e.g. 11a, etc.) and type 14 (e.g. 14a, etc.)

To a stirred solution of the corresponding carboxylic acid 10 (e.g. 10a, 10b, 10d, etc. 100 mol %) in dimethyl formamide (0.5 mL/10 mg), DIEA (300 mol %) and 2-(1H-benzotriazole-1-yl)-1,1,3,3-tetramethyluronium tetrafluoroborate (TBTU, 200 mol %) were added followed by the corresponding amine (300 mol %) and the mixture was stirred overnight at room temperature. The volatiles were removed under reduced pressure and the residue was partitioned between EtOAc (4 mL/10 mg) and aqueous sodium hydroxide (0.1 N, 1 mL/10 mg) with vigorous stirring for 2 minutes. The two phases were separated and the organic phase was washed with water ( 1×1 mL/10 mg of 10) and brine (1×1 mL/10 mg of 10), dried over Na₂SO₄, and evaporated to a residue that was purified by column chromatography using a gradient of 0 to 20% methanol in EtOAc to give the corresponding amide type 11 and 14 namely,

(R)-trans-4-[N′-(tert-Butyloxycarbonyl)butan-1′-amino]-N-(4″-pyridyl) cyclohexane Carboxamide [(1′R)-11b]: 18 mg (69%) of the corresponding amide 11b was obtained as a white solid: mp=194-196° C.; ¹H NMR (CD₃OD) δ 0.93 (m, 3H, J=6.66), 1.03-1.69 (m, 18 H), 1.83-2.99 (m, 4H), 2.35 (m, 1H), 3.35 (m, 1H), 7.64 (dd, 2H, J=1.33, 5.03), 8.37 (d, 2H, J=5.56);

(S)-trans-4-[N′-(tert-Butyloxycarbonyl)butan-1′-amino]-N-(4″-pyridyl) cyclohexane Carboxamide [(1′S)-11b]: 127 mg (71%) of the corresponding amide 11b was obtained as a white solid; HRMS calcd for C₂₁H₃₄N₃O₃ [(MH)⁺]: 376.2600, found: 376.2593;

(R)-trans-4-[N′-(tert-Butyloxycarbonyl)ethan-1′-amino]-N-(4″-pyridyl)cyclohexane Carboxamide [(1′R)-11a]: 250 mg (79%) of the corresponding amide 11a was obtained as a white solid: mp=176-178° C.; ¹H (CDCl₃): δ 0.95-1.65 (m, 17H), 1.80-2.27 (m, 5H), 3.49 (m, 1H), 4.40 (d, 1H, J=9.24), 7.52 (d, 2H, J=6.15), 7.98 (br s, 1H), 8.47 (d, 2H, J=6.17);

(S)-trans-4-[N′-(tert-Butyloxycarbonyl)ethan-1′-amino]-N-(4″-pyridyl) cyclohexane Carboxamide [(1′S)-11a]: 321 mg (80%) of the corresponding amide 11a was obtained as a white solid;

(R,S)-trans-4-[N′-(Methyloxycarbonyl)but-3′-en-1′-amino]-N-(4″-pyridyl)cyclohexane Carboxamide [(R,S)-11d): 17 mg (86%) of the amide 11d was obtained as an off-white solid: mp: 174-175° C.; HRMS calcd for C₁₈H₂₅N₃O₃ (M⁺): 331.1896, found: 331.1897; ¹H NMR (CD₃OD) δ 0.1.15 (m, 2H), 1.38-1.69 (m, 3H), 1.81-2.02 (m, 4H), 2.14 (m, 1H), 2.33 (m, 2H), 3.45 (m, 1H), 3.62 (s, 3H), 5.05 (m, 2H), 5.79 (m, 1H), 7.65 (dd, 2H,J=1.40,5.00), 8.37 (d, 2H,J=4.90);

(R)-trans-4-[N′-(Methyloxycarbonyl)but-3′-en-1′-amino]-N-(4″-pyridyl)cyclohexane Carboxamide [(R)-11d): 17 mg (85%) of the amide 11d was obtained as an off-white solid;

(S)-trans-4-[N′-(Methyloxycarbonyl)but-3′-en-1′-amino]-N-(4″-pyridyl)cyclohexane Carboxamide [(S)-11d): 22 mg (85%) of the amide 11d was obtained as an off-white solid;

(R,S)-trans-4-[(N′-tert-Butyloxycarbonyl-N′-methyl)but-3′-en-1′-amino]cyclohexane Carboxamide [(R,S)-11c]. 106 mg (81%) of the corresponding amide 11c was obtained as an off-white solid: HRMS calcd for C₂₂H₃₄N₃O₃ [(MH)⁺]: 388.2600, found: 388.2592; ¹H NMR (CDCl₃) showed a mixture of rotamers. Characteristic signals are as follows: δ 0.80-2.53 (m, 22H), 2.59 (m, 3H), 5.01 (m, 2H), 5.63 (m, 1H), 7.57 (d, 2H, J=5.16), 8.39 (d, 2H, J=5.43);

(R,S)-trans-4-[(N′-tert-Butyloxycarbonyl-N′-benzyl)but-3′-en-1′-amino]cyclohexane Carboxamide [(R,S)-11e]. 138 mg (83%) of the corresponding amide 11e was obtained as a white solid: HRMS calcd for C₂₈H₃₈N₃O₃ [(MH)⁺]: 464.2913, found: 464.2922; ¹H NMR (CDCl₃) showed a mixture of rotamers. Characteristic signals are as follows: δ 0.77-2.40 (m, 22H), 4.15-4.30 (2H), 4.85-4.97 (m, 2H), 5.45-5.67 (m, 1H), 7.10-7.30 (m, 5H), 7.53 (m, 2H), 8.38 (m, 2H), 9.37-9.58 (m, 1H);

(R,S)-trans-4-[N′-(Methyloxycarbonyl)but-3′-en-1′-amino]-N-[2″-(3′″-indolyl)ethyl]cyclohexane Carboxamide [(R,S)-14a]: 13 mg (85%) of the amide 14a was obtained as a white solid: HRMS calcd for C₂₃H₃₂N₃O₃ [(MH)⁺]: 398.2444, found: 398.2437; ¹H NMR (CD₃OD) δ 1.04 (m, 2H), 1.39 (m, 3H), 1.79 (m, 4H), 2.10 (m, 2H), 2.30 (m, 1H), 2.93 (t, 2H, J=7.22), 3.42 (m, 3H), 3.61 (s, 3H), 5.04 (m, 2H), 5.80 (m, 1H), 6.95-7.10 (m, 3H), 7.32 (d, 1H, J=8.11), 7.55 (d, 1H, J=8.10);

(R,S)-trans-4-[N′-(Methyloxycarbonyl)but-3′-en-1′-amino]-N-[(3″-pyridyl)methyl]cyclohexane Carboxamide [(R,S)-14b]: 9 mg (67%) of the amide 14b was obtained as a white solid: HRMS calcd for C₉H₂₈N₃O₃ [(MH)⁺]: 346.2131, found: 346.2129; ¹H NMR (CD₃OD) δ 1.08 (m, 2H), 1.42 (m, 3H), 1.86 (m, 4H), 2.14 (m, 2H), 2.31 (m, 1H), 3.42 (m, 1H), 3.61 (s, 3H), 4.39 (s, 2H), 5.04 (m, 2H), 5.77 (m, 1H), 7.40 (m, 1H), 7.75 (d, 1H, J=7.92), 8.43 (m, 2H);

(R,S)-trans-4-[N′-(Methyloxycarbonyl)but-3′-en-1′-amino]-N-[2″-(2′″-pyridyl)ethyl]cyclohexane Carboxamide [(RS)-14c]: 11 mg (79%) of the amide 14c was obtained as a white solid: HRMS calcd for C₂₀H₃₀N₃O₃ [(MH)⁺]: 360.2287, found: 360.2274; ¹H NMR (CD₃OD) δ 1.04 (m, 2H), 1.39 (m, 3H), 1.82 (m, 4H), 2.10 (m, 2H), 2.29 (m, 1H), 2.96 (t, 2H, J=7.02), 3.41 (m, 1H), 3.51 (t, 2H, J=7.04), 3.61 (s, 3H), 5.05 (m, 2H), 5.77 (m, 1H), 7.30 (m, 2H), 7.78 (m, 1H), 8.46 (d, 1H, J=4.89);

(R,S)-trans-4-[N′-(Methyloxycarbonyl)but-3′-en-1′-amino]-N-[4″-(N″-benzyl)piperidyl]cyclohexane Carboxamide [(R,S)-14d]: 16 mg (95%) of the amide 14d was obtained as a white solid: HRMS calcd for C₂₅H₃₈N₃O₃ [(MH)⁺]: 428.2913, found: 428.2913; ¹H NMR (CD₃OD) δ 1.05 (m, 2H), 1.46 (m, 5H), 1.81 (m, 6H), 2.12 (m, 4H), 2.28 (m, 1H), 2.88 (m, 2H), 3.42 (m, 1H), 3.52 (s, 2H), 3.61 (m, 4H), 5.04 (m, 2H), 5.76 (m, 1H), 7.30 (m, 5H);

(R,S)-trans-4-[N′-(Methyloxycarbonyl)but-3′-en-1′-amino]-N-(3″-pyridyl)cyclohexane Carboxamide [(R,S)-14e]: 9 mg (69%) of the amide 14e was obtained as a white solid: HRMS calcd for C₁₈H₂₅N₃O₃ (M⁺): 331.1896, found: 331.1899; ¹H NMR (CD₃OD) δ 1.13 (m, 2H), 1.40-1.62 (m, 3H), 1.93 (m, 4H), 2.15 (m, 1H), 2.34 (m, 2H), 3.45 (m, 1H), 3.62 (s, 3H), 5.06 (m, 2H), 5.79 (m, 1H), 7.38 (m, 1H), 8.12 (m, 1H), 8.24 (m, 1H), 8.73 (d, 1H, J=2.02);

(R,S)-trans-4-1[N′-ethyloxycarbonyl)but-3′-en-1′-amino]-N-(3″-quinolyl)cyclohexane Carboxamide [(R,S)-14f]: 7 mg (46%) of the amide 14f was obtained as a white solid: HRMS calcd for C₂₂H₂₇N₃O₃ (M⁺): 381.2052, found: 381.2046; ¹H NMR (CD₃OD) δ 1.16 (m, 2H), 1.40-1.65 (m, 3H), 1.92 (m, 2H), 2.02 (m, 2H), 2.17 (m, 1H), 2.35 (m, 2H), 3.46 (m, 1H), 3.63 (s, 3H), 5.07 (m, 2H), 5.80 (m, 1H), 7.57 (m, 1H), 7.66 (m, 1H), 7.86 (m, 1H), 7.97 (m, 1H), 8.69 (d, 1H, J=1.90), 8.90 (d, 1H, J=2.47);

(R,S)-trans-4-[N-(Methyloxycarbonyl)but-3′-en-1′-amino]-N-(5″-isoquinolyl)cyclohexane Carboxamide [(R,S)-14 g]: 5 mg (33%) of the amide 14 g was obtained as a white solid: HRMS calcd for C₂₂H₂₇N₃O₃ (M⁺): 381.2052, found: 381.2053; ¹H NMR (CD₃OD) δ 1.20 (m, 2H), 1.47 (m, 1H), 1.63 (m, 2H), 1.95 (m, 2H), 2.12 (m, 3H), 2.35 (m, 1H), 2.53 (m, 1H), 3.48 (m, 1H), 3.61 (s, 3H), 5.05 (m 2H), 5.80 (m, 1H), 7.70 (m, 1H), 7.87 (d, 1H, J=6.09), 7.91 (d, 1H, J=6.81), 8.01 (d, 1H, J=8.18), 8.47 (d, 1H, J=6.03), 9.26 (s, 1H);

(R,S)-trans-4-[N′-(Methyloxycarbonyl)but-3′-en-1′-amino]-N-(6″-quinolyl)cyclohexane Carboxamide [(R,S)-14h]: 7 mg (46%) of the amide 14h was obtained as a white solid: HRMS calcd for C₂₂H₂₇N₃O₃ (M⁺): 381.2052, found: 381.2054; ¹H NMR (CD₃OD) δ 1.16 (m, 2H), 1.46 (m, 1H), 1.58 (m, 2H), 1.84-2.03 (m, 4H), 2.18 (m, 1H), 2.39 (m, 2H), 3.46 (m, 1H), 3.63 (s, 3H), 5.07 (m, 2H), 5.80 (m, 1H), 7.50 (m, 1H), 7.80 (m, 1H), 7.96 (m, 1H), 8.27 (m, 1H, J=8.04), 8.37 (d, 1H, J=2.21), 8.74 (m, 1H);

(R,S)-trans-4-[N′-(Methyloxycarbonyl)but-3′-en-1′-amino]-N-[4″-(dimethylamino)benzyl]cyclohexane Carboxamide 1(R,S)-14i]: 6 mg (40%) of the amide 14i was obtained as a white solid: HRMS calcd for C₂₂H₃₃N₃O₃ (M⁺): 387.2522, found: 387.2522;

(R,S)-trans-4-[N′-(Methyloxycarbonyl)but-3′-en-1′-amino]-N-(4″-quinaldyl)cyclohexane Carboxamide [(R,S)-14j]: 5 mg (33%) of the amide 14j was obtained as a white solid: HRMS calcd for C₂₃H₂₉N₃O₃ (M⁺): 395.2209, found: 395.2224;

(R,S)-trans-4-[N′-(Methyloxycarbonyl)but-3′-en-1′-amino]-N-(5″-indolyl)cyclohexane Carboxamide [(R,S)-14k]: 10 mg (69%) of the amide 14k was obtained as a white solid: HRMS calcd for C₂₁H₂₇N₃O₃ (M⁺): 369.2052, found: 369.2068;

(R,S)-trans-4-[N′-(Methyloxycarbonyl)but-3′-en-1′-amino]-N-[(4″-pyridyl)methyl]cyclohexane Carboxamide [(R,S)-14l]: 10 mg (74%) of the amide 14l was obtained as a white solid: HRMS calcd for C₁₉H₂₇N₃O₃ (M⁺): 345.2052, found: 345.2039; ¹H NMR (CD₃OD) δ 1.10 (m, 2H), 1.47 (m, 3H), 1.90 (m, 4H), 2.10-2.37 (m, 3H), 3.43 (m, 1H), 3.61 (s, 3H), 4.40 (s, 2H), 5.05 (m, 2H), 5.78 (m, 1H), 7.32 (d, 2H, J=5.58), 8.46 (d, 2H, J=2.98); and

(R,S)-trans-4-[N′-(Methyloxycarbonyl)but-3′-en-1′-amino]-N-(6″-puryl)cyclohexane Carboxamide [(R,S)-14m]: 15 mg (14%) of the corresponding amide 14m was obtained as a yellowish solid: HRMS calcd for C₁₈H₂₅N₆O₃ [(MH)⁺]: 373.1988, found: 373.1999; ¹H NMR (CDCl₃) showed a mixture of rotamers. Characteristic signals are as follows: δ δ 1.03-2.60 (m, 13H), 3.68 (m, 3H), 4.57 (m, 1H), 5.10 (d, 2H, J=11.99), 5.77 (m, 1H), 8.36 (s, 1H), 8.72 (s, 1H), 10.07 (br s, 1H), 11.75 (br s, 1H).

### EXAMPLE 46

### General Procedure for Preparation of Dihydrochloride Salts 12a 12b, 12c and 12e

A solution of the corresponding amide 11 (e.g. 11a, 11b, etc. 100 mol %) in dry DCM (1 mL/10 mg) was cooled to 0° C. in an ice bath and treated with a stream of gaseous hydrochloric acid bubbles for 15 minutes. The ice bath was removed and the reaction was allowed to reach ambient temperature with stirring for 30 minutes. The volatiles were removed and the residue was triturated with diethyl ether and dried to give the corresponding dihydrochloric salt which was directly tested without further purification, namely

(R)-trans-4-(Butan-1′-amino)-N-(4″-pyridyl)cyclohexane Carboxamide Dihydrochloride [BA-1001, (R)-12b dihydrochloride]: 13 mg of the corresponding dihydrochloric acid salt 12b was obtained as an off-white solid: HRMS calcd for C₁₆H₂₆NO₃ [(MH)⁺]: 276.2075, found: 276.2089; ¹H NMR (CD₃OD) δ 1.01 (t, 3H, J=7.19), 1.23-1.80 (m, 9H), 1.89 (2H), 2.10 (m, 2H), 2.57 (m, 1H), 3.09 (m, 1H), 8.21 (d, 2H, J=7.31), 8.61 (d, 2H, J=7.27);

(S)-trans-4-(Butan-1′-amino)-N-(4″-pyridyl)cyclohexane Carboxamide Dihydrochloride [(BA-1002), (S)-12b dihydrochloride]: 102 mg of the corresponding dihydrochloric acid salt 12b was obtained as an off-white solid: ¹H NMR (CD₃OD) δ 1.01 (t, 3H, J=7.22), 1.25-1.81 (m, 9H), 1.90 (2H), 2.10 (m, 2H), 2.57 (m, 1H), 3.09 (m, 1H), 8.21 (d, 2H, J=7.26), 8.61 (d, 2H, J=7.23);

(R)-trans-4-(Ethan-1′-amino)-N-(4″-pyridyl)cyclohexane Carboxamide Dihydrochloride [Y-27632, (R)-12a dihydrochloride]: 135 mg of the corresponding dihydrochloric acid salt 12a was obtained as an off-white solid: HRMS calcd for C₁₄H₂₂N₃O [(MH)⁺]: 248.1762, found: 248.1769; ¹H (CD₃OD) δ 1.18-1.38 (m and d, 5H, J=6.73), 1.60 (m, 3H), 1.94 (m, 2H), 2.10 (m, 2H), 2.53 (m, 1H), 3.18 (m, 1H), 8.19 (br s, 2H), 8.61 (br s, 2H);

(S)-trans-4-(Ethan-1′-amino)-N-(4″-pyridyl)cyclohexane Carboxamide Dihydrochloride [(S)-12a dihydrochloride]: 207 mg of the corresponding dihydrochloric acid salt 12a was obtained as an off-white solid;

(R,S)-trans-4-[(N′-methyl)-but-3′-en-1′-amino]-N-(4″-pyridyl)cyclohexane Carboxamide Dihydrochloride [BA-1028, (R,S)-12c]: 75 mg of the corresponding dihydrochloric acid salt 12d was obtained as a white solid: HRMS calcd for C₁₇H₂₆N₃O₁ [(MH)⁺]: 288.2076, found: 288.2086; ¹H NMR (CD₃OD) showed a mixture of rotamers. Characteristic signals are as follows: δ 1.30-2.60 (m, 12H), 2.74 (m, 3H), 3.15 (m, 1H), 5.30 (m, 2H), 5.85 (m, 1H), 8.22 (d, 2H, J=6.80), 8.61 (d, 2H, J=6.99); and

(R,S)-trans-4-[(N′-benzyl)-but-3′-en-1′-amino]-N-(4″-pyridyl)cyclohexane Carboxamide Dihydrochloride [BA-1029, (R,S-12e]: 75 mg of the corresponding dihydrochloric acid salt 12e was obtained as a white solid: HRMS calcd for C₂₃H₃₀N₃O₁ [(MH) ]: 364.2389, found: 364.2386; ¹H NMR (CD₃OD) showed a mixture of rotamers. Characteristic signals are as follows: δ 1.3-2.18 (m, 9H), 2.58 (m, 3H), 3.18 (m, 1H), 4.35 (m, 2H), 5.30 (m, 2H), 5.87 (m, 1H), 7.40-7.60 (m, 5H), 8.22 (d, 2H, J=6.34), 8.61 (d, 2H, J=6.53).

### EXAMPLE 47

### General Procedure for Preparation of Dihydrochloride Salts 12d and 15 (e.g. 15a to 15m.)

A stirred, room temperature solution of the corresponding amide (11d and 14 (e.g. 14a to 14m), 100 mol %) in dry chloroform (1 mL/10 mg), was treated with iodotrimethylsilane (500 mol %), stirred overnight at room temperature and evaporated under reduce pressure to a residue, that was dissolved in methanol (1 mL/10 mg), stirred for 5 minutes and evaporated to dryness. The residue was dissolved in iso-propanol (1 mL/10 mg), cooled to 0° C., and treated with a solution of hydrochloric acid in iso-propanol (1-2 M, 500 mol %). After removal of the volatiles under reduce pressure, the residue was triturated with diethyl ether to give the corresponding dihydrochloric salt which was directly tested without further purification, namely

(R,S-trans-4-(But-3′-en-1′-amino)-N-(4″-pyridyl)cyclohexane Carboxamide Dihydrochloride [BA-1003, (R,S)-12d]: 11 mg of the corresponding dihydrochloric acid salt 12c was obtained as a yellowish solid: HRMS calcd for C₁₆H₂₃N₃O₁ (M⁺): 273.1841, found: 273.1844; ¹H NMR (CD₃OD) δ 1.29 (m, 2H), 1.53-1.81 (m, 3H), 1.93 (m, 2H), 2.12 (m, 2H), 2.40 (m, 1H), 2.55 (m, 2H), 3.19 (m, 1H), 5.29 (m, 2H), 5.85 (m, 1H), 8.23 (d, 2H, J=6.08), 8.62 (d, 2H, J=6.41);

(R)-trans-4-(But-3′-en-1′-amino)-N-(4″-pyridyl)cyclohexane Carboxamide Dihydrochloride [BA-1016, (R)-12d]: 23 mg of the corresponding dihydrochloric acid salt 12c was obtained as a yellowish solid;

(S)-trans-4-(But-3′-en-1′-amino)-N-(4″-pyridyl)cyclohexane Carboxamide Dihydrochloride [BA-1017, (S)-12d]: 20 mg of the corresponding dihydrochloric acid salt 12c was obtained as a yellowish solid;

(R,S)-trans-4-(But-3′-en-1′-amino)-N-[2″-(3′″-indolyl)ethyl]cyclohexane Carboxamide Dihydrochloride [BA-1004, (R,S)-15a]: 13 mg of the dihydrochloric acid salt 15a was obtained as a yellowish solid: HRMS calcd for C₂₁H₂₉N₃O₁ (M⁺): 339.2311, found: 339.2316; ¹H NMR (CD₃OD) δ 1.18 (m, 2H), 1.48(m, 2H), 1.64 (m, 1H), 1.85 (m, 4H), 2.15 (m, 1H), 2.35 (m, 1H), 2.50 (m, 1H), 2.95 (t, 2H, J=7.17), 3.11 (m, 1H), 3.48 (t, 2H, J=7.32), 5.28 (m, 2H), 5.82 (m, 1H), 6.98 (m, 1H), 7.09 (m, 2H), 7.33 (d, 1H, J=8.11), 7.57 (d, 1H, J=7.33);

(R,S)-trans-4-(But-3′-en-1′-amino)-N-[(3″-pyridyl)methyl]cyclohexane Carboxamide Dihydrochloride [BA-1005, (R,S)-15b]: 10 mg of the dihydrochloric acid salt 15b was obtained as a yellowish solid: HRMS calcd for C₁₇H₂₅N₃O₁ (M⁺): 287.1998, found: 287.1991; ¹H NMR (CD₃OD) δ 1.26 (m, 2H), 1.52 (m, 2H), 1.68 (m, 1H), 1.90 (m, 2H), 2.02 (m, 2H), 2.37 (m, 2H), 2.50 (m, 1H), 3.15 (m, 1H), 4.58 (s, 2H), 5.27 (m, 2H), 5.83 (m, 1H), 8.10 (m, 1H), 8.59 (d, 1H, J=8.14), 8.80 (m, 2H);

(R,S)-trans4-(But-3′-en-1′-amino)-N-[2″-(2′″-pyridyl)ethyl]cyclohexane Carboxamide Dihydrochloride [BA-1006, (R,S)-15c]: 14 mg of the dihydrochloric acid salt 15c was obtained as a yellowish solid: HRMS calcd for C₁₈H₂₇N₃O₁ (M⁺): 301.2154, found: 301.2159; ¹H NMR (CD₃OD) δ 1.21 (m, 2H), 1.39 (m, 2H), 1.63 (m, 1H), 1.84 (m, 4H), 2.16 (m, 1H), 2.36 (m, 1H), 2.47 (m, 1H), 3.12 (m, 1H), 3.26 (t, 2H, J=6.46), 3.64 (t, 2H, J=6.56), 5.25 (m, 2H), 5.81 (m, 1H), 7.97 (m, 2H), 8.56 (m, 1H), 8.78 (m, 1H).;

(R,S)-trans-4-(But-3′-en-1′-amino)-N-[4″-(N″-benzyl)piperidyl]-cyclohexane Carboxamide Dihydrochloride [BA-1007, (R,S)-15d]: 17 mg of the dihydrochloric acid salt 15d was obtained as a yellowish solid: HRMS calcd for C₂₃H₃₅N₃O₁ (M⁺): 369.2780, found: 369.2792; ¹H NMR (CD₃OD) δ 1.25 (m, 2H), 1.49 (m, 2H), 1.60-2.30 (m, 11H), 2.36 (m, 1H), 2.50 (m, 1H), 3.15 (m, 2H), 3.54 (m, 2H), 3.90 (m, 1H), 4.36 (s, 2H), 5.26 (m, 2H), 5.83 (m, 1H), 7.55 (m, 5H);

(R,S)-trans-4-(But-3′-en-1′-amino)-N-(3″-pyridyl)cyclohexane Carboxamide Dihydrochloride [BA-1008, (R,S)-15e]: 12 mg of the dihydrochloric acid salt 15e was obtained as a yellowish solid: HRMS calcd for C₁₆H₂₃N₃O₁ (M⁺): 273.1841, found: 273.1831; ¹H NMR (CD₃OD) δ 1.31 (m, 2H), 1.55-1.77 (m, 3H), 1.93 (m, 2H), 2.12 (m, 2H), 2.40 (m, 1H), 2.54 (m, 2H), 3.19 (m, 1H), 5.28 (m, 2H), 5.84 (m, 1H), 8.04 (m, 1H), 8.58 (m, 2H), 9.46 (d, 1H, J=2.30);

(R,)-trans-4-(But-3′-en-1′-amino)-N-(3″-quinolyl)cyclohexane Carboxamide Dihydrochloride [BA-1009, (R,S)-15f]: 6 mg of the dihydrochloric acid salt 15f was obtained as a yellowish solid: HRMS calcd for C₂₀H₂₅N₃O₁ (M⁺): 323.1998, found: 323.1993; ¹H NMR (CD₃OD) δ 1.33 (m, 2H), 1.65 (m, 3H), 1.95 (m, 2H), 2.14 (m, 2H), 2.41 (m, 1H), 2.56 (m, 2H), 3.19 (m, 1H), 5.33 (m, 2H), 5.85 (m, 1H), 7.93 (m, 1H), 8.07 (m, 1H), 8.18 (d, 1H, J=8.59), 8.27 (d, 1H, J=8.29), 9.17 (d, 1H, J=2.03), 9.66 (d, 1H, J=2.35);

(R,S)-trans-4-(But-3′-en-1′-amino)-N-(5″-isoquinolyl)cyclohexane Carboxamide Dihydrochloride [BA-1010, (R,S)-15g]: 5 mg of the dihydrochloric acid salt 15g was obtained as a yellowish solid: m/z (MAB) 323.2 (M⁺); ¹H NMR (CD₃OD) δ 1.32 (m, 2H), 1.60-2.18 (m, 7H), 2.2.35 (m, 1H), 2.55 (m, 1H), 2.70 (m, 1H), 3.18 (m, 1H), 5.30 (m, 2H), 5.85 (m, 1H), 8.07 (m, 1H), 8.30 (m, 1H), 8.40 (m, 1H), 8.49 (m, 1H), 8.62 (m, 1H), 9.83 (m, 1H);

(R,S)-trans-4-(But-3′-en-1′-amino)-N-(6″-quinolyl)cyclohexane Carboxamide Dihydrochloride [BA-1011, (R,S)-15h]: 8 mg of the dihydrochloric acid salt 15h was obtained as a yellowish solid: HRMS calcd for C₂₀H₂₅N₃O₁ (M⁺): 323.1998, found: 323.1990; ¹H NMR (CD₃OD) δ 1.32 (m, 2H), 1.60-2.18 (m, 7H), 2.42 (m, 1H), 2.55 (m, 2H), 3.18 (m, 1H), 5.31 (m, 2H), 5.83 (m, 1H), 8.06 (m, 1H), 8.20 (d, 1H, J=9.25), 8.29 (m, 1H), 8.81 (d, 1H, J=1.97),9.10 (m, 2H);

(R,S)-trans-4-(But-3′-en-1′-amino)-N-[4∝1 -(dimethylamino)-benzyl]cyclohexane Carboxamide Dihydrochloride [BA-1012, (R,S)-15i]: 6 mg of the dihydrochloric acid salt 15i was obtained as a yellowish solid: m/z (MAB) 329.2 (M⁺); ¹H NMR (CD₃OD) δ 1.10-2.43 (m, 12H), 3.20 (m, 1H), 3.25 (s, 6H), 4.40 (s, 2H), 5.25 (m, 2H), 5.82 (m, 1H), 7.50 (m, 2H), 7.60 (m, 2H);

(R,S-trans-4-(But-3′-en-1′-amino)-N-(4″-quinaldyl)cyclohexane Carboxamide Dihydrochloride [BA-1013, (R,S)-15j]: 8 mg of the dihydrochloric acid salt 15j was obtained as a yellowish solid: HRMS calcd for C₂₁H₂₇N₃O₁ (M⁺): 337.2154, found: 337.2153; ¹H NMR (CD₃OD) δ 1.10-2.43 (m, 12H), 2.90 (s, 3H), 3.19 (m, 1H), 5.25 (m, 2H), 5.82 (m, 1H), 7.90 (m, 1H), 8.07 (m, 2H), 8.70 (m, 2H);

(R,S)-trans-4-(But-3′-en-1′-amino)-N-(5″-indolyl)cyclohexane Carboxamide Dihydrochloride [BA-1014, (R,S)-15k]: 13 mg of the dihydrochloric acid salt 15k was obtained as a yellowish solid: HRMS calcd for Cl₉H₂₅N₃O₁ (M⁺): 311.1998, found: 311.2007; ¹H NMR (CD₃OD) δ 1.10-2.50 (m, 12H), 3.17 (m, 1H), 5.23 (m, 2H), 5.80 (m, 1H), 7.00-8.00 (m, 5H);

(R,S)-trans-4-(But-3′-en-1′-amino)-N-[(4″-pyridyl)methyl]cyclohexane Carboxamide Dihydrochloride [BA-1015, (R,-151]: 10 mg of the dihydrochloric acid salt 151 was obtained as a yellowish solid: HRMS calcd for C₁₇H₂₅N₃O₁ (M⁺): 287.1998, found: 287.2000; ¹H NMR (CD₃OD) δ 1.28 (m, 2H), 1.56 (m, 2H), 1.70 (m, 1H), 1.90 (m, 2H), 2.07 (m, 2H), 2.40 (m, 2H), 2.51 (m, 1H), 3.15 (m, 1H), 4.67 (s, 2H), 5.27 (m, 2H), 5.81 (m, 1H), 7.99 (d, 2H, J=6.69), 8.80 (d, 2H, J=6.75); and

(R,S)-trans-4-(But-3′-en-1′-amino)-N-(6″-puryl)cyclohexane Carboxamide Dihydrochloride [BA-1031, (R,S-15m]: 8 mg of the dihydrochloric acid salt 15m was obtained as a yellowish solid: HRMS calcd for C₁₆H₂₃N₆O₁ [(MH)⁺]: 315.1933, found: 315.1932; ¹H NMR (CD₃OD) δ 1.13-2.77 (m, 12H), 3.20 (m, 1H), 5.30 (m, 2H), 5.85 (m, 1H), 8.88 (s, 1H), 9.12 (s, 1H).

### EXAMPLE 48

### Preparation of Ethyl 4-ketocyclohexanecarboxylate (16)

To a stirred solution of pyridinium chlorochromate (9.48 g, 44.0 mmol) in DCM (50 mL) at 4° C. was added a solution of ethyl 4-hydroxycyclohexanecarboxylate (5.00 g, 29.0 mmol). The mixture was stirred at 4° C. for 2 hours than heated at reflux and stirred for another 4 hours. The reaction was cooled at room temperature, filtered on Celite™ and the filtrate was evaporated to a residue which was purified by column chromatography using a gradient of 25 to 45% of EtOAc in hexane as eluant to give 4.93 g (100%) of the ketone 16 as a clear oil: HRMS calcd for C₉H₁₄O₃ (M⁺): 170.0943, found: 170.0938; ¹H NMR (CDCl₃) δ 1.26 (t, 3H, J=7.15), 2.02 (m, 2H), 2.19 (m, 2H), 2.34 (m, 2H), 2.44 (m, 2H), 2.73 (m, 1H), 4.16 (q, 2H, J=7.13).

### EXAMPLE 49

### Preparation of Ethyl 4-[N′-(tert-butyloxycarbonyl)-hydrazono]cyclohexane Carboxylate (17)

To a stirred solution of ethyl 4-ketocyclohexanecarboxylate (16, 3.04 g, 17.9 mmol) in toluene (25 mL) was added tert-butyloxycarbonylcarbazide (2.36 g, 17.9 mmol). The mixture was stirred 5 minutes then was allow to stand at room temperature for 24 hours. The reaction was treated with Na₂SO₄ (10 g), stirred at room temperature for 3 hours and filtered. The filtrate was evaporated to dryness to give quantitatively the hydrazone 17 as an oil: ¹H NMR (CDCl₃) δ 1.26 (t, 3H, J=7.13), 1.50 (s, 9H), 1.76 (m, 2H), 2.02 (m, 3H), 2.27 (m, 1H), 2.57 (m, 3H), 4.14 (q, 2H, J=7.13), 7.56 (s, 1H).

### EXAMPLE 50

### Preparation of Ethyl 4-[N′-(tert-butyloxycarbonyl)-hydrazino]cyclohexane Carboxylate (18)

To a solution of ethyl 4-[N′-(tert-butyloxycarbonyl)hydrazono]cyclohexane carboxylate (17, 5.06 g, 17.8 mmol) in THF (20 mL) was added sodium cyanoborohydride (1.40 g, 22.3 mmol) followed by bromocresol green (5 mg). The mixture was vigorously stirred at room temperature then treated over 2 hours with a solution of p-toluenesulfonic acid (3.06 g, 17.8 mmol) in THF (20 mL) in order to keep a green color in the reaction mixture. The reaction was partitioned between EtOAc (100 mL) and brine (50 mL). The phases were separated and the aqueous phase was extracted with EtOAc (1×25 mL). The combined organic phases were washed with NaHCO₃ sat. (2×50 mL) and brine (1×50 mL), dried over Na₂SO₄ and evaporated to dryness. The residue was suspended in dioxane (25 mL) then slowly treated with aqueous sodium hydroxide (1 N, 17 mL). The mixture was stirred for 5 minutes at room temperature then was partitioned between EtOAc (125 mL) and water (20 mL). The two phases were separated and the aqueous phase was extracted with EtOAc (1×25 mL). The combined organic phases were washed with brine (1×50 mL), dried over Na₂SO₄ and evaporated to a residue that was purified by column chromatography using a gradient of 30 to 50% EtOAc in hexane as eluant. The first product eluted was the cis-isomer of the hydrazine 18 (1.41 g, 28%) followed by the trans-isomer (1.75 g, 34%), namely

cis-Ethyl 4-[N′-(tert-butyloxycarbonyl)hydrazino]cyclohexane Carboxylate (18). m/z (MAB) 286.2 (M⁺); ¹H NMR (CDCl₃) δ 1.20 (t, 3H, J=7.12), 1.40-1.62 (m, 15H), 1.95 (m, 2H), 2.36 (m, 1H), 2.95 (m, 1H), 3.78 (br s, 1H), 4.08 (q, 2H, J=7.13), 6.27 (br s, 1H); and

trans-Ethyl 4-[N′-(tert-butyloxycarbonyl)hydrazino]cyclohexane Carboxylate (18). m/z (MAB) 286.2 (M⁺); ¹H NMR (CDCl₃) δ 1.04 (m, 2H), 1.18 (t, 3H, J=7.12), 1.35-1.48 (m, 11H), 1.90 (m, 4H), 2.16 (m, 1H), 2.75 (m, 1H), 3.94 (br s, 1H), 4.05 (q, 2H, J=7.13), 6.42 (br s, 1H).

### EXAMPLE 51

### General Procedure for the Preparation of Alkylhydrazines of type 19

To a stirred solution of either cis- or trans-ethyl 4-(tert-butyloxycarbonylhydrazino)-cyclohexane carboxylate (18, 100 mol %) in acetonitrile (1.5 mL/100 mg of 18) was added 37% aqueous formaldehyde or the corresponding aldehyde (500 mol %) followed by sodium cyanoborohydride (200 mol %). The mixture was stirred at room temperature for 15 minutes then acetic acid was added in order to reach a pH of around 6 (about 30 μL/100 mg of 18). The mixture was stirred at room temperature for 5 hours. In the course of reaction, small aliquots of acetic acid (5 82 L/100 mg of 18) were added to keep the pH around 6. The reaction was then partitioned between water (2 mL/100 mg of 18) and EtOAc (10 mL/100 mg of 18). The two phases were separated and the aqueous phase was extracted with EtOAc (2×2 mL/100 mg of 18). The combined organic phases were washed with brine (1×5 mL/100 mg of 18), dried over Na₂SO₄, and evaporated to a residue that was purified by column chromatography using a gradient of 30 to 40% EtOAc in hexane as eluant to give the corresponding hydrazine 19, namely

cis-Ethyl 4-[N′-(tert-butyloxycarbonyl)-N-(methyl)hydrazino]cyclohexane Carboxylate (19a). 54 mg (69%) of the corresponding hydrazine 19a was obtained as an oil: m/z (FAB) 301.1 [(MH)⁺];

trans-Ethyl 4-[N′-(tert-butyloxycarbonyl)-N-(methyl)hydrazino]-cyclohexane Carboxylate (19a). 43 mg (35%) of the corresponding hydrazine 19a was obtained as an oil: m/z (FAB) 301.1 [(MH)⁺];

cis-Ethyl 4-[N′-(tert-butyloxycarbonyl)-N-(propyl)hydrazino]cyclohexane Carboxylate (19b). 126 mg (63%) of the corresponding hydrazine 19b was obtained as an oil: HRMS calcd for C₁₇H₃₂N₂O₄ (M⁺): 328.2362, found: 328.2355;

trans-Ethyl 4-[N′-(tert-butyloxycarbonyl)-N-(propyl)hydrazino]cyclohexane Carboxylate (19b). 82 mg (71%) of the corresponding hydrazine 19b was obtained as an oil: HRMS calcd for C₁₇H₃₃N₂O₄ [(MH)⁺]: 329.2440, found: 329.2428;

cis-Ethyl 4-{N′-(tert-butyloxycarbonyl)-N-[3′-(methyl)butyl]hydrazino}-cyclohexane Carboxylate (19c). 133 mg (75%) of the corresponding hydrazine 19c was obtained as an oil: HRMS calcd for C₁₉H₃₆N₂O₄ (M⁺): 356.2675, found: 356.2680;

trans-Ethyl 4-{N′-(tert-butyloxycarbonyl)-N-13′-(methyl)butyl]hydrazino}-cyclohexane Carboxylate (19c). 85 mg (71%) of the corresponding hydrazine 19c was obtained as an oil: HRMS calcd for Cl₉H₃₇N₂O₄ [(MH)⁺]: 357.2753, found: 357.2763.

cis-Ethyl 4-{N′-(tert-butyloxycarbonyl)-N-[1′-(methyl)ethyl]hydrazino}cyclohexane Carboxylate (19d). 47 mg (19%) of the corresponding hydrazine 19d was obtained as an oil: HRMS calcd for C₁₇H₃₂N₂O₄ (M⁺): 328.2362, found: 328.2354;

trans-Ethyl 4-{N′-(tert-butyloxycarbonyl)-N-[1′-(methyl)ethyl]hydrazino}cyclohexane Carboxylate (19d). 12 mg (10%) of the corresponding hydrazine 19d was obtained as an oil: HRMS calcd for C₁₇H₃₂N₂O₄ [(MH)⁺]: 329.2440, found: 329.2456;

cis-Ethyl 4-[N′-(tert-butyloxycarbonyl)-N-(benzyl)hydrazino]cyclohexane Carboxylate (19e). 78 mg (27%) of the corresponding hydrazine 19e was obtained as an oil: HRMS calcd for C₂₁H₃₂N₂O₄ (M⁺): 376.2362, found: 376.2347;

trans-Ethyl 4-[N-(tert-butyloxycarbonyl)-N-(benzyl)hydrazino]cyclohexane Carboxylate (19e). 40 mg (31%) of the corresponding hydrazine 19e was obtained as an oil: HRMS calcd for C₂₁H₃₃N₂O₄ [(MH)⁺]: 377.2440, found: 377.2431;

trans-Ethyl 4-{N′-(tert-butyloxycarbonyl)-N-12′(phenyl)ethyl]hydrazino}cyclohexane Carboxylate (19f). 228 mg (84%) of the corresponding hydrazine 19f was obtained as an oil: HRMS calcd for C₂₂H₃₄N₂O₄ (M⁺): 390.2519, found: 390.2524;

trans-Ethyl 4-{N′-(tert-butyloxycarbonyl)-N-[2′,2′-(diphenyl)ethyl]-hydrazino}cyclohexane Carboxylate (19g). 170 mg (52%) of the corresponding hydrazine 19g was obtained as an oil: HRMS calcd for C₂₈H₃₉N₂O₄ [(MH)⁺]: 467.2910, found: 467.2908;

trans-Ethyl 4-{N′-(tert-butyloxycarbonyl)-N-[4′-(benzyloxy)benzyl]-hydrazino}cyclohexane Carboxylate (19h). 218 mg (65%) of the corresponding hydrazine 19h was obtained as an oil: ¹H NMR (CDCl₃) δ 1.20-1.53 (m, 16H), 2.05 (m, 4H), 2.22 (m, 1H), 2.70 (m, 1H), 3.70-3.90 (br s, 2H), 4.11 (q, 2H, J=7.13), 5.00-5.50 (m, 3H), 6.90-7.47 (m, 9H);

trans-Ethyl 4-{N′-(tert-butyloxycarbonyl)-N-[(cyclohexyl)methyl]-hydrazino}cyclohexane Carboxylate (19i). 235 mg (88%) of the corresponding hydrazine 19i was obtained as an oil: ¹H NMR (CDCl₃) δ 0.70-0.85 (m, 2H), 1.00-2.00 (m, 29H), 2.13 (m, 1H), 2.27-2.53 (m, 3H), 4.05 (q, 2H, J=7.13), 5.20 (br s, 1H);

trans-Ethyl 4-[N-(tert-butyloxycarbonyl)-N-(octyl)hydrazino]cyclohexane Carboxylate (19j). 222 mg (80%) of the corresponding hydrazine 19j was obtained as an oil: 0.80 (m, 5H), 1.00-1.55 (m, 25H), 1.75 (m, 1H), 1.90 (m, 4H), 2.11 (m, 1H), 2.55 (m, 3H), 4.05 (q, 2H, J=712), 5.25 (s, 1H); and

1,4-trans-2′,3′-trans-Ethyl 4-{N′-(tert-butyloxycarbonyl)-N-[3′-(phenyl)prop-2′-enyl]hydrazino}cyclohexane Carboxylate (19k). 128 mg (45%) of the corresponding hydrazine 19k was obtained as an oil: HRMS calcd for C₂₃H₃₄N₂O₄ (M⁺): 402.2519, found: 402.2517.

### EXAMPLE 52

### General Procedure for the Preparation of Carboxylic Acids of type 20

To a stirred solution of the corresponding hydrazine 19 (100 mol %) in dioxane (1.7 mL/100 mg of 19) was slowly added aqueous sodium hydroxide (1 N, 600 mol %). The mixture was stirred at room temperature for 2 hours then was acidified to pH 3-4 with 2 N aqueous hydrochloric acid. The volatiles were removed under reduced pressure to give quantitatively the corresponding carboxylic acid 20 (which was used in the next step (example 53) without further purification), namely

trans-4-[N′-(tert-Butyloxycarbonyl)-N-(propyl)hydrazino]cyclohexane Carboxylic Acid (20b). m/z (FAB) 301.3 [(MH)⁺];

trans-4-{N′-(tert-Butyloxycarbonyl)-N-[3′-(methyl)butyl]hydrazino} cyclohexane Carboxylic Acid (20c). m/z (FAB) 329.4 [(MH)⁺];

trans-4-{N′-(tert-Butyloxycarbonyl)-N-[1′-(methyl)ethyl]hydrazino}-cyclohexane Carboxylic Acid (20d). m/z (FAB) 301.3 [(MH)⁺]; and

trans-4-[N′-(tert-Butyloxycarbonyl)-N-(benzyl)hydrazino]cyclohexane Carboxylate (19e). m/z (FAB) 349.3 [(MH)⁺];

### EXAMPLE 53

### General Procedure for Preparation of Amides 21

To a stirred solution of the corresponding carboxylic acid 20 (100 mol %) in dimethyl formamide (0.5 mL/10 mg), DIEA (600 mol %) and 2-(1H-benzotriazole-1-yl)-1,1,3,3-tetramethyluronium tetrafluoroborate (TBTU, 400 mol %) were added followed by the 4-aminopyridine (400 mol %) and the mixture was stirred overnight at room temperature. The volatiles were removed under reduced pressure and the residue was partitioned between EtOAc (4 mL/10 mg of 20) and aqueous sodium hydroxide (0.1 N, 1 mL/10 mg of 20) with vigorous stirring for 2 minutes. The two phases were separated and the organic phase was washed with water ( 1×1 mL/10 mg of 20) and brine (1×1 mL/10 mg of 20), dried over Na₂SO₄, and evaporated to a residu that was purified by column chromatography using a gradient of 0 to 10% methanol in EtOAc to give the corresponding amide 21, namely

cis-4-[N″-(tert-Butyloxycarbonyl)-N-(methyl)hydrazino]-N′-(4′″-pyridyl)cyclohexane Carboxamide (21a). 41 mg (86%) of the corresponding amide 21a was obtained as an off-white solid: HRMS calcd for C₁₈H₂₉N₄O₃ [(MH)⁺]: 349.2240, found: 349.2231;

trans-4-[N″-(tert-Butyloxycarbonyl)-N′-(methyl)hydrazino]-N-(4′″-pyridyl)cyclohexane Carboxamide (21a). 36 mg (91%) of the corresponding amide 21a was obtained as an off-white solid: HRMS calcd for C₁₈H₂₉N₄O₃ [(MH)⁺]: 349.2240, found: 349.2249;

cis-4-[N″-(tert-Butyloxycarbonyl)-N′-(propyl)hydrazino]-N-(4′″-pyridyl)cyclohexane Carboxamide (21b). 85 mg (64%) of the corresponding amide 21b was obtained as an off-white solid: HRMS calcd for C₂₀H₃₃N₄O₃ [(MH)⁺]: 377.2553, found: 377.2541;

trans-4-[N″-(tert-Butyloxycarbonyl)-N′-(propyl)hydrazino]-N-(4′″-pyridyl)cyclohexane Carboxamide (21b). 45 mg (75%) of the corresponding amide 21b was obtained as an off-white solid: HRMS calcd for C₂₀H₃₂N₄O₃ (M⁺): 376.2474, found: 376.2481;

cis-4-{N″-(tert-Butyloxycarbonyl)-N′-[3′-(methyl)butyl]hydrazino}-N-(4′″-pyridyl)cyclohexane Carboxamide (21c). 93 mg (74%) of the corresponding amide 21c was obtained as an off-white solid: HRMS calcd for C₂₂H₃₇N₄O₃ [(MH)⁺]: 405.2866, found: 405.2852;

trans-4-{N″-(tert-Butyloxycarbonyl)-N′-[3′-(methyl)butyl]hydrazino]-N-(4′″-pyridyl)cyclohexane Carboxamide (21c). 64 mg (76%) of the corresponding amide 21c was obtained as an off-white solid: HRMS calcd for C₂₂H₃₆N₄O₃ (M⁺): 404.2787, found: 404.2790;

cis-4-{N′-(tert-Butyloxycarbonyl)-N′-[1′-(methyl)ethyl]hydrazino}-N-(4′″-pyridyl)cyclohexane Carboxamide (21d). 26 mg (55%) of the corresponding amide 21d was obtained as an off-white solid: HRMS calcd for C₂₀H₃₃N₄O₃ [(MH)⁺]: 377.2553, found: 377.2544;

trans-4-{N′-(tert-Butyloxycarbonyl)-N′-[1′-(methyl)ethyl]hydrazino]-N-(4′″-pyridyl)cyclohexane Carboxamide (21d). 24 mg (84%) of the corresponding amide 21d was obtained as an off-white solid: HRMS calcd for C₂₀H₃₂N₄O₃ (M⁺): 376.2474, found: 376.2461;

cis-4-[N′-(tert-Butyloxycarbonyl)-N′-(benzyl)hydrazino]-N-(4′″-pyridyl)cyclohexane Carboxamide (21e). 52 mg (60%) of the corresponding amide 21e was obtained as an off-white solid:. HRMS calcd for C₂₄H₃₃N₄O₃ [(MH)⁺]: 425.2553, found: 425.2569;

trans-4-[N″-(tert-Butyloxycarbonyl)-N′-(benzyl)hydrazino]-N-(4′″-pyridyl)cyclohexane Carboxamide (21e). 52 mg (92%) of the corresponding amide 21e was obtained as an off-white solid:. HRMS calcd for C₂₄H₃₂N₄O₃ (M⁺): 424.2474, found: 424.2492;

trans-4-{N′-(tert-Butyloxycarbonyl)-N′-[2′-(phenyl)ethyl]hydrazino}-N-(4′″-pyridyl)cyclohexane Carboxamide (21f). 126 mg (60%) of the corresponding amide 21f was obtained as an off-white solid: ¹H NMR (CDCl₃) δ 1.30 (m, 2H), 1.4-1.63 (m, 1H), 1.97 (m, 4H), 2.30 (m, 1H), 1.62 (m, 1H), 2.75 (m, 2H), 2.88 (m, 2H), 7.10-7.28 (m, 5H), 7.64 (d, 2H, J=6.28), 8.35 (d, 2H, J=4.56);

trans-4-{N″-(tert-Butyloxycarbonyl)-N′-[2′,2′-(diphenyl)ethyl]hydrazino}-N-(4′″-pyridyl)cyclohexane Carboxamide (21g). 127 mg (85%) of the corresponding amide 21g was obtained as an off-white solid: ¹H NMR (CDCl₃) δ 1.05-1.63 (m, 13H), 1.95 (m, 4H), 2.20 (m, 1H), 2.70 (m, 1H), 3.05-3.48 (m, 2H), 4.18 (t, 1H, J=7.04), 5.38-5.57 (m, 1H), 7.10-7.30 (m, 10H), 7.56 (d, 2H, J=5.54), 8.41 (d, 2H, J=5.13), 9.02-9.23 (m, 1H);

trans-4-{N″-(tert-Butyloxycarbonyl)-N′-[4′-(benzyloxy)benzyl]hydrazino}-N-(4′″-pyridyl)cyclohexane Carboxamide (21h). 72 mg (39%) of the corresponding amide 21h was obtained as an off-white solid: ¹H NMR (CDCl₃) δ 1.33 (m, 11H), 1.62 (m, 2H), 2.00-2.10 (m, 4H), 2.28 (m, 1H), 2.72 (m, 1H), 3.70-3.95 (m, 2H), 4.97-5.45 (m, 3H), 6.90 (d, 2H, J=8.30), 7.18-7.43 (m, 7H), 7.54 (d, 2H, J=6.32), 8.47 (m, 3H);

trans-4-{N″-(tert-Butyloxycarbonyl)-N′-[(cyclohexyl)methyl]hydrazino}-N-(4′″-pyridyl)cyclohexane Carboxamide (21i). 147 mg (82%) of the corresponding amide 21i was obtained as an off-white solid: ¹H NMR (CDCl₃) δ 0.80 (m, 2H), 1.00-1.23 (m, 5H), 1.28-1.70 (m, 15H), 1.73-2.03 (m, 6H), 2.23 (m, 1H), 2.32-2.54 (m, 3H), 5.00-5.40 (br s, 1H), 7.57 (m, 2H), 8.41 (d, 2H, J=6.25), 9.34 (s, 1H);

trans-4-[N″-(tert-Butyloxycarbonyl)-N′-(octyl)hydrazino]-N-(4′-pyridyl)cyclohexane Carboxamide (21j). 98 mg (35%) of the corresponding amide 21j was obtained as an off-white solid: ¹H NMR (CDCl₃) δ 0.84 (t, 3H, J=6.9), 1.10-1.33 (m, 12H), 1.35-1.50 (m, 11H), 1.58 (m, 2H), 1.87-2.07 (m, 4H), 2.26 (m, 1H), 2.45-2.70 (m, 3H), 5.35 (br s, 1H), 7.58 (d, 2H, J=6.26), 8.42 (d, 2H, J=6.20), 9.17 (s, 1H);and

1,4-trans-2′,3′-trans-4-{N″-(tert-Butyloxycarbonyl)-N′-[3′-(phenyl)prop-2′-enyl])hydrazino}-N-(4′″-pyridyl)cyclohexane Carboxamide (21k). 12 mg (16%) of the corresponding amide 21k was obtained as an off-white solid: ¹H NMR (CDCl₃) δ 1.20-1.50 (m, 11H), 1.62 (2H), 2.08 (m, 4H), 2.28 (m, 1H), 2.70 (m, 1H), 3.58 (m, 2H), 5.1-5.45 (br s, 1H), 6.25 (m, 1H), 6.50 (s, 1H), 7.18-7.35 (m, 5H), 7.61 (d, 2H, J=5.58), 8.43 (m, 3H).

### EXAMPLE 54

### General Procedure for Preparation of Dihydrochloride Salts of Type 22

A solution of the corresponding amide 21 (100 mol %) in dry DCM (1 mL/10 mg of 21) was cooled to 0° C. in an ice bath and treated with a stream of gaseous hydrochloric acid bubbles for 30 minutes. The ice bath was removed and the reaction was allowed to reach ambient temperature with stirring for 30 minutes. The reaction was cooled to 0° C. in an ice bath and treated a second time with a stream of gaseous hydrochloric acid bubbles for 15 minutes. The ice bath was removed and the reaction was allowed to reach ambient temperature with stirring for 30 minutes. The volatiles were removed and the residue was triturated with diethyl ether and dried to give the corresponding dihydrochloric acid salt, namely

cis-4-[N′-(Methyl)hydrazino]-N-(4″-pyridyl)cyclohexane Carboxamide Dihydrochloride [BA-1018, 22a dihydrochloride]: 39 mg of the corresponding dihydrochloric acid salt 22a was obtained as a white solid: HRMS calcd for C₁₃H₂₁N₄O₁ [(MH)⁺]: 249.1715, found: 249.1704; ⁺); ¹H NMR (CD₃OD) δ 1.80 (m, 2H), 2.00 (m, 4H), 2.17 (m, 2H), 2.87 (m, 1H), 2.93 (s, 3H), 3.20 (m, 1H), 8.22 (d, 2H, J=7.35), 8.62 (d, 2H, J=7.35);

trans-4-[N′-(Methyl)hydrazino]-N-(4″-pyridyl)cyclohexane Carboxamide Dihydrochloride [BA-1019, 22a dihydrochloride]: 25 mg of the corresponding dihydrochloric acid salt 22a was obtained as a white solid: HRMS calcd for C₁₃H₂₁N₄O₁ [(MH)⁺]: 249.1715, found: 249.1707; ¹H NMR (CD₃OD) δ 1.65 (m, 4H), 2.20 (m, 4H), 2.55 (m, 1H), 2.97 (s, 3H), 3.20 (m, 1H), 8.22 (d, 2H, J=7.36), 8.62 (d, 2H, J=7.36);

cis-4-[N′-(Propyl)hydrazino]-N-(4″-pyridyl)cyclohexane Carboxamide Dihydrochloride [BA-1024, 22b dihydrochloride]: 58 mg of the corresponding dihydrochloric acid salt 22b was obtained as a white solid: HRMS calcd for C₁₅H₂₅N₄O₁ [(MH)⁺]: 277.2028, found: 277.2033; ¹H NMR (CD₃OD) δ 1.03 (t, 3H, J=7.41); 1.80 (m, 4H), 2.00 (m, 4H), 2.27 (m, 2H), 2.91 (m, 1H), 3.18 (m, 2H), 3.35 (m, 1H), 8.22 (d, 2H, J=7.34), 8.62 (dd, 2H, J=0.62, 7.24);

trans-4-[N′-(Propyl)hydrazino]-N-(4″-pyridyl)cyclohexane Carboxamide Dihydrochloride [BA-1020, 22b dihydrochloride]: 35 mg of the corresponding dihydrochloric acid salt 22b was obtained as a white solid: HRMS calcd for C₁₅H₂₅N₄O₁ [(MH)⁺]: 277.2028, found: 277.2039; ¹H NMR (CD₃OD) δ 1.04 (t, 3H, J=7.39), 1.60-2.00 (m, 6H), 2.20 (m, 4H), 2.58 (m, 1H), 3.05-3.20 (m, 3H), 8.21 (d, 2H, J=7.33), 8.61 (d, 2H, J=7.29);

cis-4-{N′-[3′-(Methyl)butyl]hydrazino}-N-(4″-pyridyl)cyclohexane Carboxamide Dihydrochloride [BA-1025, 22c dihydrochloride]: 68 mg of the corresponding dihydrochloric acid salt 22c was obtained as a white solid: HRMS calcd for C₁₇H₂₉N₄O₁ [(MH)⁺]: 305.2341, found: 305.2335; ¹H NMR (CD₃OD) δ 0.99 (d, 6H, J=6.52),1.50-2.35 (m, 1H), 2.88 (m, 1H), 3.10-3.40 (m, 3H), 8.23 (d, 2H, J=7.07), 8.63 (d, 2H, J=6.80);

trans-4-{N′-[3′-(Methyl)butyl]hydrazino}-N-(4″-pyridyl)cyclohexane Carboxamide Dihydrochloride [BA-1021, 22c dihydrochloride]: 43 mg of the corresponding dihydrochloric acid salt 22c was obtained as a white solid: HRMS calcd for C₁₇H₂₉N₄O₁ [(MH)⁺]: 305.2341, found: 305.2348; ¹H NMR (CD₃OD) δ 1.00 (d, 6H, J=6.28), 1.50-1.90 (m, 7H), 2.20 (m, 4H), 2.60 (m, 1H), 3.13-3.40 (m, 3H), 8.22 (d, 2H, J=7.37), 8.62 (d, 2H, J=7.35);

cis-4-{N′-[1′-(Methyl)ethyl]hydrazino}-N-(4″-pyridyl)cyclohexane Carboxamide Dihydrochloride [BA-1026, 22d dihydrochloride]: 15 mg of the corresponding dihydrochloric acid salt 22d was obtained as a white solid: HRMS calcd for C₁₅H₂₅N₄O₁ [(MH)⁺]: 277.2028, found: 277.2033; ¹H NMR (CD₃OD) δ 1.35 (d, 6H, J=6.07), 1.85 (m, 2H), 1.95-2.15 (m, 4H), 2.27 (m, 2H), 2.90 (m, 1H), 3.38 (m, 1H), 3.80 (m, 1H), 8.22 (d, 2H, J=7.40), 8.62 (d, 2H, J=7.36);

trans-4-{N′-[1′-(Methyl)ethyl]hydrazino}-N-(4″-pyridyl)cyclohexane Carboxamide Dihydrochloride [BA-1022, 22d dihydrochloride]: 17 mg of the corresponding dihydrochloric acid salt 22d was obtained as a white solid: m/z (FAB) 277 [(MH)⁺]; ¹H NMR (CD₃OD) δ 1.27-1.43 (m, 6H, J=1.57-1.80 (m, 4H), 2.20 (m, 3H), 2.40 (m, 1H), 2.55 (m, 1H), 3.40 (m, 1H), 3.80 (m, 1H), 8.20 (d, 2H, J=7.39), 8.61 (d, 2H, J=7.35);

cis-4-[N′-(Benzyl)hydrazino]-N-(4″-pyridyl)cyclohexane Carboxamide Dihydrochloride [BA-1027, 22e dihydrochloride]: 32 mg of the corresponding dihydrochloric acid salt 22e was obtained as a white solid: HRMS calcd for C₁₉H₂₅N₄O₁ [(MH)⁺]: 325.2028, found: 325.2036; ¹H NMR (CD₃OD) δ 1.70-2.37 (m, 8H), 2.85 (m, 1H), 3.00-3.40 (m, 1H), 4.00-4.65 (m, 2H), 7.35-7.60 (m, 5H), 8.22 (d, 2H, J=7.27), 8.62 (d, 2H, J=7.28);

trans-4-[N′-(Benzyl)hydrazino]-N-(4″-pyridyl)cyclohexane Carboxamide Dihydrochloride [BA-1023, 22e dihydrochloride]: 33 mg of the corresponding dihydrochloric acid salt 22e was obtained as a white solid: m/z (FAB) 325 [(MH)⁺]; ¹H NMR (CD₃OD) δ 1.60-1.80 (m, 4H), 2.20 (m, 4H), 2.60 (m, 1H), 3.20 (m, 1H), 4.27 (m, 2H), 7.20-7.35 (m, 5H), 8.21 (d, 2H, J=7.36), 8.61 (d, 2H, J=7.35);

trans-4-{N′-[2′-(Phenyl)ethyl]hydrazino}-N-(4′″-pyridyl)cyclohexane Carboxamide Dihydrochloride [BA-1033, 22f dihydrochloride]). 100 mg (89%) of the corresponding dihydrochloric acid salt 22f was obtained as an off-white solid: m/z (FAB) 339.2 [(MH)⁺]; ¹H NMR (CD₃OD) δ 1.70 (m, 4H), 2.20 (m, 4H), 2.60 (m, 1H), 3.00-3.55 (m, 5H), 5.20-5.40 (m, 5H), 8.22 (d, 2H, J=7.26), 8.61 (d, 2H, J=7.20);

trans-4-{N′-[2′,2′-(Diphenyl)ethyl]hydrazino}-N-(4′″-pyridyl)cyclohexane Carboxamide Dihydrochloride [BA-1034, 22g dihydrochloride]). 89 mg (81%) of the corresponding dihydrochloric acid salt 22g was obtained as an off-white solid: m/z (FAB) 415.2 [(MH)⁺]; ¹H NMR (CD₃OD) δ 1.60 (m, 4H), 1.95-2.18 (m, 4H), 2.57 (m, 1H), 3.12 (m 1H), 3.70 (m, 2H), 4.47 (m, 1H), 7.18-7.42 (m, 10H), 8.22 (d, 2H, J=7.30), 8.61 (d, 2H, J=7.34);

trans-4-{N′-[4′-(Benzyloxy)benzyl]hydrazino}-N-(4′″-pyridyl)cyclohexane Carboxamide Dihydrochloride [BA-1035, 22h dihydrochloride]). 53 mg (87%) of the corresponding dihydrochloric acid salt 22h was obtained as an off-white solid: m/z (FAB) 431.2 [(MH)⁺]; ¹H NMR (CD₃OD) δ 1.70 (m, 4H), 2.20 (m, 4H), 2.60 (m, 1H), 3.27 (m, 1H), 4.28 (m, 2H), 5.15 (s, 2H), 7.10 (d, 2H, J=8.71), 7.28-7.47 (m, 7H), 8.22 (d, 2H, J=7.35), 8.62 (d, 2H, J=7.34);

trans-4-{N′-[(Cyclohexyl)methyl]hydrazino}-N-(4′″-pyridyl)cyclohexane Carboxamide Dihydrochloride [BA-1036, 22i dihydrochloride]). 112 mg (79%) of the corresponding dihydrochloric acid salt 22i was obtained as an off-white solid: m/z (MAB) 330.3 (M⁺); ¹H NMR (CD₃OD) δ 1.05 (m, 2H), 1.18-1.43 (m, 3H), 1.60-1.95 (m, 10 H), 2.18 (m, 4H), 2.62 (m, 1H), 2.98 (m, 2H), 3.25 (m, 1H), 8.24 (d, 2H, J=7.25), 8.62 (d, 2H, J=7.25);

trans-4-[N′-(Octyl)hydrazino]-N-(4′″-pyridyl)cyclohexane Carboxamide Dihydrochloride [BA-1037, 22j dihydrochloride]). 79 mg (89%) of the corresponding dihydrochloric acid salt 22j was obtained as an off-white solid: m/z (FAB) 347.3 [(MH)⁺]; ¹H NMR (CD₃OD) δ 0.92 (t, 3H, J=7.05), 1.25-1.45 (m, 11H), 1.60-1.85 (m, 6H), 2.20 (m, 4H), 2.57 (m, 1H), 3.20 (m, 2H), 8.23 (d, 2H, J=7.34), 8.62 (d, 2H, J=7.30); and;

1,4-trans-2′,3′-trans-4-{N′-[3′-(Phenyl)prop-2′-enyl]hydrazino}-N-(4′″-pyridyl)cyclohexane Carboxamide Dihydrochloride [BA-1038, 22k dihydrochloride]). 8 mg (79%) of the corresponding dihydrochloric acid salt 22k was obtained as an off-white solid: m/z (FAB) 351.2 [(MH)⁺]; ¹H NMR (CD₃OD) δ 1.50-2.35 ( m, 9H), 2.55 (m, 1H), 3.90-4.20 (m, 2H), 6.40 (m, 1H), 6.93 (m, 1H), 7.35 (m, 3H), 7.52 (m, 2H), 8.21 (d, 2H, J=7.21), 8.62 (d, 2H, J=7.21).

### EXAMPLE 55

### trans-4-[N′-(Octyl)hydrazino]-N-(4′″-pyridyl)cyclohexane Carboxamide [BA-1037, 22j])

To a stirred solution of trans-4-[N′-(octyl)hydrazino]-N-(4′″-pyridyl)cyclohexane carboxamide dihydrochloride (BA-1037, 22j dihydrochloride, 10 mg, 0.024 mmol) in distilled water (1 mL) was added at room temperature sodium carbonate (50 mg) and the mixture was stirred for 2 minutes. Chloroform (10 mL) was added and the mixture was stirred for 15 minutes at room temperature. The two phases were separated and the aqueous phase was extracted with chloroform (2×2 mL). The combined organic phases were dried over Na₂SO₄ and evaporated to dryness to give quantitatively the free base 22j as an oil: ¹H NMR (CD₃OD) δ 0.85 (t, 3H, J=7.03), 1.20-1.42 (m, 12H), 1.43-1.60 (m, 4H), 1.95 (m, 4H), 2.28 (m, 1H), 2.45 (m, 1H), 2.57 (m, 1H), 3.10 (m, 1H), 7.55 (d, 2H, J=7.32), 8.29 (d, 2H, J=7.32).

Preparation of Human Rho Kinase (ROK) Expressed in COS Cells

ROK has been prepared and cDNAs cloned from a number of sources and the cloning of human p160-ROK cDNA (p160-ROCK1) has been reported (Ishizaki et al., 1996, EMBO J. 15: 1885; U.S. Pat. No. 5,906,819). Overexpression in mammalian cells provides a convenient, easily renewed source of ROK activity. ROK is available as a clone in pCAG-myc-p160 (Ishizaki et al., 1997, FEBS Lett. 404: 118). The myc tag in this expression plasmid allows for purification using immunological techniques. Transfection-quality DNA is prepared from E. coli (DH5α or XL1-Blue) containing the pCAG-myc-p160 ^{myc-727} (Ishizaki et al., 1997) using a midi-kit (Qiagen). This construct expresses ROK activity in a constitutive fashion and yields a polypeptide of about 98 kDa. COS cells are plated and grown overnight. The expression vector DNA is introduced using lipofectamine (Qiagen), followed by an 18 hour incubation. The following steps are performed on ice. The transfected cells are washed with pre-cooled PBS, then lysed with buffer containing a cocktail of protease and phosphatase inhibitors (20 mM Tris-HCl (pH=7.5), 1 mM EDTA, 1 mM EGTA, 5 mM MgCl₂, 25 mM NaF, 10 mM β glycerophosphate, 5 mM sodium pyrophosphate, 0.2 mM phenylmethylsulfonyl fluoride, 2 mM dithiothreitol, 0.2 mM sodium vanadate, 0.05% Triton X-100, 0.1 μM calyculin A). The cells are scraped into 1.5 mL Eppendorf tubes and centrifuged at 10,000 g for 10 min. The supernatant is transferred to a fresh tube and the pellet discarded. Anti-myc antibody (9E10; Sigma #M5546) is added, and the tube rotated for 2 hours at 4° C. Protein G-Sepharose (Sigma, #P3296) prewashed in lysis buffer is added and the incubation and rotation continued for another 2 hours. The suspension is then centrifuged at 1,000 g for 5 min and the pellet is washed 3 times with lysis buffer and once with ROK kinase buffer (50 mM Hepes-NaOH (pH=7.4), 10 mM MgCl₂, 5 mM MnCl₂, 2 mM dithiothreiol, 0.02% Brij 35). The pellet is suspended in ROK kinase buffer to give a standard enzyme product of immobilized ROK.

ROK can also be purchased commercially.

Testing for Inhibition of Rho Kinase (ROK) Activity

The ability of compounds to inhibit ROK activity may be tested in a cell-free assay system using recombinant ROK, radiaoactive ATP, and Myelin basic Protein (MBP). MBP is a highly phosphorylated protein, is inexpensive to buy in purified form, is phosphorylated by ROK, and is used as the assay substrate for phosphorylation. Recombinant ROK has been prepared from a number of sources. Overexpression in mammalian cells provides a convenient, easily renewed source of ROK activity. Measurement of Rho-associated kinase (ROK) activity is important to determine the potency of novel inhibitors. MBP is a substrate for ROK and a number of other protein kinases, making it useful both to quantitate ROK activity and to indicate the potency and specificity of novel inhibitors for ROK. Conditions can be adjusted for analysis of other substrates. The assay is modified as necessary to provide optimal buffer and incubation conditions to check IC50 (Inactivation concentration 50%) values for other protein kinases to provide an index of specificity for ROK kinase. Other protein kinases that are used to assess specificity for ROK include PKCα, PKA, PKN, and MLCK.

Example Kinase Assay

ROCK II was assayed in 20 mM MOPS, pH 7.2, 25 mM β-glycerophosphate, 5 mM EGTA, 1 mM sodium orthovanadate, 1 mM dithiothreitol with dephosphorylated myelin basic protein (MBP, 0.2 mg/ml) as substrate with or without BA-1016 or BA-1017. Assays were performed for 30 min at 30° C. in 50 μl using [γ-³²P] ATP. The concentrations of ATP and magnesium chloride were 100 μM and 75 mM respectively. Assays were initiated by adding Mg²⁺/ATP and terminated by spotting 40 μl of each reaction onto phosphocellulose paper (P81 paper, Whatman), followed by washes in 0.75% phosphoric acid to remove ATP and then dried, put in scintillation cocktail and counted to measure ³²P incorporation. Radioactivity is measured using a scintillation counter. Percent activity for a particular concentration of inhibitor is calculated as 100*(a−b)/(c−b), where a=cpm (enzyme+inhibitor), b=cpm (autophosphorylation of substrate and kinase) and c=cpm (enzyme−inhibitor). A dose-response chart was prepared for each inhibitor, then an IC₅₀ (inhibitor concentration at 50% inhibition) determination was made to measure the potency of inhibition. A plot of the log of concentration of test inhibitor (x axis) and the percent inhibition of kinase activity (y axis) was prepared. The curve was interpolated to estimate the amount of each compound necessary for 50% inhibition. ROCK II and dephosphorylated myelin basic protein (MBP) were purchased from Upstate (Lake Placid, N.Y.). ATP is from Boehringer Mannheim and [γ-³²P] ATP is from Perkin-Elmer.

Referring to FIG. 3 this figure illustrates inhibition of ROCKII activity by BA-1016 and BA-1017. Inhibition of ROCK activity is plotted as a function of BA-1016 (triangles) or BA-1017 (squares) concentration. Experiments were done in duplicate. BA-1016 was assayed in two separate experiments, each in duplicate, and the mean±SEM is shown.

Referring to FIGS. 8 and 9 inhibiton of ROCKII activity and determination of IC50 may also be done throught the use of known (i.e. commercial) inhibition assays that use recombinant human ROCKII.

Quick Bioassay

To test the ability of ROK inhibitors to promote neurite growth on inhibitory substrates, we use a bioassay that is quick (4 hours) and reliable. This assay tests the ability of a compound to promote neurite outgrowth in tissue culture. The advantage of using this assay as a first screen is that any compounds that are toxic or that are unable to pass the plasma membrane are eliminated at the earliest stage of testing.

Example: Bioassay to Determine Growth Promoting Activity

A rapid bioassay is used to determine the effect of a test compounds on the stimulation of neurite growth in vitro. A neuronal cell line, NG108-15 (ATCC HB-12317), is maintained in culture in Dulbecco's minimal essential medium (DMEM) supplemented with 10% fetal bovine serum, Penicillin/Streptomycin and HAT supplement (Gibco/BRL). For the bioassay, the cells are collected by trypsinisation and ressuspended in DMEM supplemented with 5% FBS, Penicillin/Streptomycin, HAT supplement and 0.25 mg/ml cAMP, adjusted to 1.0×10⁴ cells/ml. The cells are plated into wells of a 96 well plate at 100 μls (1000 cells)/well. Cells are incubated 4 h at 37° C. and 5% CO₂ in presence of small molecules or cethrin at a concentration of 1000 cells per well of a 96-wells plate in a final volume of 100 ul.

After incubation, cells are fixed by adding 351 μls 16% PFA and 5.4 μls 2.5% glutaraldehyde to the media in each well. The wells are stained with cresyl violet 0.05% , 100 μls/well for 15 min. Cells with neurites (length≧one cell body) are counted using an inverted light microscope. The % neurite outgrowth is determined by calculating the number of cells with neurites over the total number of counted cells. See FIG. 1.

Inhibition Assay on Inhibitory Substrate

PC-12 cells (ATCC CRL-1721) typically extend neurites in response to NGF, but when plated on inhibitory substrates, this outgrowth is inhibited and the cells remain round. We tested the ability of the new compound to grow neurites when plated on inhibitory substrates.

BA-1003, BA-1016 and BA-1017 were all able to overcome growth inhibition by MAG. BA-1017 was effective at the lowest concentration tested, 0.31 uM. on MAG substrates cell remain round and are unable to extend substrates. When BA-1003, BA-1016 or BA-1017 was added to the culture medium the cells differentiated and grew long neurite. MAG is an inhibitory protein present in the CNS, and the receptor to MAG is a common receptor shared by the other major myelin-derived inhibitors Nogo and Oligodendrocyte myelin glycoprotein (Science 297:1132 (2002)). The MAG receptor is called Nogo-66 receptor, or NgR. The results that show that BA-1003, BA-1016 and BA-1017 can overcome growth inhibition by MAG indicate that these compounds can overcome growth inhibition by nogo-66 receptor—dependent mechanisms. These results indicate that the compounds should be effective in promoting growth in the central nervous system, which has a growth inhibitory environment.

BioAssay on Growth Inhibitory Myelin-associated Glycoprotein (MAG) Substrates

We used PC12 cells obtained from the American Type Culture Collection. PC12 cells were grown in Dulbecco's modified eagle's medium (DMEM) with 10% horse serum and 5% fetal bovine serum. To test compounds for the ability to overcome growth inhibition, PC12 cells were collected by detaching with trypsin-EDTA (0.05%), then resuspended in DMEM, 1% FBS, and 50 ng/ml nerve growth factor before plating on MAG substrates. MAG used for substrates was purified from myelin after extraction in 1% octylglucoside and separation by ion exchange chromatography (McKerracher et al. 1994, Neuron 13:805-811). Test substrates were prepared as uniform substrates in 96-well plates by drying overnight in the laminar flow hood (Nalge Nunc, Naperville, Ill.). Plates were precoated with polylysine (100 μg/ml) for 3 hours at 37° C., then washed and dried approx. 1 hour. MAG was prepared as a substrate by drying down 8 μg of protein overnight. After plating on the MAG substrate, the cells were grown at 37 C. for two days in the presence or absence of test compound (BA1003, BA1016, BA1017) to allow neurite growth. Polylysine substrates were used as a positive control. Quantitative analysis of neurite outgrowth was with the aid of Northern Eclipse software (Empix Imaging, Mississauga, Ontario). Data analysis and statistics were with Microsoft Excel.

Referring to FIG. 4 there is shown a graph illustrative of experiments performed in triplicate testing the ability of BA-1003, BA-1016 and BA-1017 to overcome growth inhibition by MAG. PC12 cells were plated on MAG substrates alone (MAG) or MAG substrates in the presence of 0.31 uM, 3.1 um or 31 um concentrations of the test compound. The numbers of neurons that grew neurites were scored, and are shown as the percentage of neurite growth.

Cell Survival After Axotomy

The retinal ganglion cell (RGC) response to injury and ischemia has been well documented (Berkelaar et al., 1994; Selles-Navarro et al., 1996; Vidal-Sanz et al., 1988; Villegas-Perez et al., 1998; Villegas-Perez et al., 1993). Transection of the optic nerve (ON) in the adult rat, as a model of fiber tract lesion in the adult mammalian CNS, results in delayed, mainly apoptotic death of 80-90% of retinal ganglion cells (RGCs) within 14 days post-lesion. Because of good surgical accessibility of the retina and the optic nerve, the retino-tectal projection represents not only a convenient model to study the molecular mechanisms underlying neuronal death but also serves as a suitable system for investigating potential neuroprotective agents in vivo.

Testing of BA-1016 to support cell survival 7 days after axotomy.

| | Average | | |
|---|---|---|---|
| | RGC | | % |
| | counts | SEM | Survival |
| Unoperated | 2286 | 57.3 | 100 |
| control | | | |
| Axotomy | 1261 | 74.04 | 55 |
| Axotomy + PBS | 1072 | 107.42 | 47 |
| Axotomy + 1 mM | 1378 | 145 | 60 |
| BA-1016 | | | |
| Axotomy + 0.1 mM | 1352 | 48 | 59 |
| BA-1016 | | | |

After optic nerve transection, many of the retinal ganglion cells (RGC) in the eye die by apoptosis. The ability of BA-1016 to support RGC cell survival after optic nerve injury was tested. A single injection of BA-1016 was made in the eye, and one week later cell survival was assessed. Three animals were examined for each treatment group. Cell survival was improved one week after BA-1016. Neurotrophic factors that are known to rescue RGCs also give improved cell surival one week after axotomy, but multiple or chronic application is needed to increase the number of surviving cells. Our results with BA-1016 indicated that multiple or chronic application may be effective to rescue injured RGCs.

Detailed Methods

Retrograde Labeling of RGCs:

Experiments were performed on adult female CD rats (180-200 g; Charles River, Canada). Animals were cared for according to the Canadian Council on Animal Care. Rats were under general anaesthesia with isofluorane connected to Moduflex Access anaesthesia machine during experimental procedures. Ophthalmic eye ointment (Polysporin) was applied to prevent corneal desiccation. RGCs were retrogradely labelled with Fluorogold (Fluorochrome, Inc. Denver, Colo., U.S.A; 2% in 0.9% NaCl containing 10% dimethyl sulfoxide) applied with a small piece of gel foam on the surface of right superior colliculus (SC). All rats were pre-labelled with Fluorogold one week prior to optic nerve lesion.

Optic Nerve Transection and Drug Administration:

One week after Fluorogold application, the left optic nerve was transected 1 mm from the eye. The optic nerve was accessed within the orbit by making an incision parasagitally in the skin covering the superior rim of the orbit bone, by means of micro scissors taking care to leave the supraorbital vein intact. Following subtotal resection or reflection of the lacrimal gland using blunt preparation, the superior extraocular muscles were spread with a small retractor or suture 6-0 silk to keep both hands free. The superior orbital contents were dissected and the rectus muscles were reflected laterally. When the optic nerve was exposed, the surrounding dura mater sheath was cut longitudinally to avoid cutting blood vessels while revealing the optic nerve. There are blood vessels on pia and optic nerve. The pia mater sheath was lifted and a lateral incision exposed the optic nerve. It was important not to cut the optic nerve before cutting the pia. When pia was cut, the optic nerve was moved gently to dislodge it from its sheath so that the scissors could be slipped under it to cut it. It was important to not pull the nerve at this point to avoid compromising the blood supply. When the optic nerve was well exposed, small scissors were slid tangentially under optic nerve, making sure to see their end on other side of the nerve, then cutting with one clean cut at 1 mm from the eye. Scissor blades were used as a reference for the 1 mm distance.

In the group of animals assigned to receive intravitreal injection after axotomy, the compounds of interest were injected into the vitreous space. The eye was punctured at the superior nasal retina area with a 30 gauge needle and then a Hamilton syringe was used to inject 10 ug in 5 microlitres of the test compound over a 1 minute period. The needle was removed after one minute. Once done, tissue adhesive (Indermil) was used to seal the overture. Lens injury was avoided because it has been demonstrated that delayed lens injury preferentially affected survival of the RGCs.

A binocular microscope was used to view the eye during injection. The skin was closed with staples (auto clips) and the integrity of the retinal vasculature was evaluated by a postoperative ophthalmoscopy using a water-covered microscope slide. Rats with compromised vasculature were not included in the experimental results. Finally the animals were returned to the cage and closely monitored until awakened

Retinal Wholemounts:

Seven days after axotomy, animals were killed by injecting an overdose of Chloral hydrate intraperitoneally and then they were fixed by perfusion with 4% paraformaldehyde (PFA), 0.1 M phosphate buffer; the eyes were remove carefully transecting the ocular muscles with scissors and forceps. The eyes were fixed in 4% PFA and the cornea was puncture to allow entrance of PFA to the posterior pole of the eye. After, the retina was separated carefully from the eye bulb and flat-mounted on glass slides incising the tissue according to the four retinal quadrants. RGCs are examined under the fluorescence microscope with an UV filter (365/420). The number of fluorescent RGCs were counted on 12 standard areas (0.45×0.35 mm each) located beside the optic nerve head and at 1.35 and 2.7 mm from the optic disc in each of the retinal quadrants.

In Vivo Results

To examine the ability of BA-1016 to promote axon regeneration in vivo, the regeneration of retinal ganglion cell axons was examined in the optic nerve after intravitrial injection of BA-1016. In these experiments, BA-1016 was injected at in the vitreous of rats immediately after optic nerve crush that transects all of the retinal ganglion cell (RGC) axons (Lehmann et al, IBID). Two weeks later the animals cholera toxin B subunit was injected in the eye to anterogradelty label the regenerating RGC axons. The next day the animals were killed perfused with saline, and the optic nerves removed for sectioning. Longitudinal sections of the optic nerve were reacted for anti-cholera toxin immunoreactivity to observe the anterogradely labeled fibers. RGC axons were observed after treatment with BA-1016 (FIG. 5), and distances of axon growth exceeded 500 um. No axon regeneration was observed in the buffer-treated controls (FIG. 6).

Detailed Methods.

Rats were anesthetized with isoflorane (2.4%) and the head shaved. To make microcrush lesions, the left optic nerve was exposed by a supraorbital approach, the optic nerve sheath slit longitudinally, the optic nerve lifted out from the sheath and crushed 1 mm from the globe by constriction with a 10.0 suture held for 60 seconds. Immediately after optic nerve crush the test solution or buffer control (phosphate buffered saline) was injected into the vitreous in the amount of 100 ug in a volume of 5 ul. After 2 weeks, all rats received an intravitreal injection of 5 μl 1% cholera toxin β subunit (CTB; List Biological Labs., Campbell, Calif.) 24 hr before perfusion with PFA. Optic nerves were dissected, post-fixed 1 hr in PFA, cryoprotected overnight in 30% sucrose and frozen at −70° C. in OCT (Canlab, Montreal, PQ). Longitudinal cryostat sections of optic nerves were cut at 14 μm and mounted on Superfrost Plus slides (Fisher, Montreal, PQ). Retinal ganglion cell axons were labeled by CTB were detected by immunohistochemistry for CTB using a goat anti-choleragenoid (List Biological Labs.), a biotinylated rabbit anti-goat (Vector Labs., Burlingame, Calif.) and DTAF-conjugated streptavidin (Jackson Labs., West Grove, Pa.) as described previously (Lehmann et al IBID)

Referring to FIG. 5, this figure illustrates a longitudinal section of an optic nerve treated with BA-1016. The site of the lesion is indicated with large arrows. Regenerating axons that extend past the lesion site are shown with small arrows.

Referring to FIG. 6, this figure illustrates a longitudinal section of a control optic nerve. Axons do not regenerate past the site of the lesion (large arrows).

FIGS. 4 to 6 illustrate that compounds in accordance with the present invention (e.g. BA-1016, BA-1017) may be used to promote axon growth on inhibitory substrates in vitro and/or in vivo.

Determining anti-proliferative effects of BA-1037 for cancer cells.

The antiproliferative effects of BA-1037 were tested by a thymidine uptake assay with several different human cancer cell lines grown in culture. The cell lines tested were HEC-1B human adnocarcinoma, SK-MEL-1 human malignant melanoma, and Caco-2 human colorectal adenocarcinoma. The cells were seeded in a 96 well plate and after 2 hours the cells were treated with test BA-1037 compound or with control solutions. The control solutions were PBS as a negative control, and with complete medium plus the DMSO vehicle (at 0.1% or 1%). BA-1037 was added at three different concentrations: 1 uM, 10, uM or 100 uM. Each control solution and test solution was plated in triplicate for each cell line. The plate was incubated at 37 C. with 5% CO₂ in a humidified atmosphere for approximately 54 hours. A volume of 0.02 ml of 3H-methy thymidine which contained 1.0 uCi was added to each well. The culture was incubated a further 18 hours. Using an automated cell harvestor the cells from each well were aspirated onto a glass microfiber filter. The cells were broken with distilled water to leave mainly the DNA on the filter. Each filter was placed in a scintillation counter (TopCount NXT). At appropriate settings for the 3H, each filter was counted for one minute. The results are expressed as CPM (counts per minute).

Cancer is characterized by the uncontrolled division of a population of cells which, most typically, leads to the formation of one or more tumors. Rho kinase is inhibited by our compounds, as shown in FIGS. 1 and FIG. 2. The small GTPase Rho is upregulated in certain cancers, such as malignant melanoma and breast cancer. Fritz et al. (Fritz et al.,(1999) Int. J. Cancer 81: 682-687) found increased protein levels in colon, breast and lung tumors. Upregulation of Rho would activate Rho kinase, and therefore, inactivation of Rho kinase is expected to reduce or cure malignancy. Many studies with the Rho kinase inhibitor have shown that inactivation of Rho kinase reduces cell migration in malignancy (eg. Sawada K, et al, Gynecol. Oncol. 2002 Dec;87(3):252-9).

FIG. 11 shows that BA-1037 tested at concentrations of 100 uM, 10 uM, and 1 uM was able to reduce cell proliferation of SK-MEL-1 cells, a human malignant melanoma cell line. The highest concentration tested showed a complete arrest of cell proliferation. Malignant melanoma cells are highly proliferate, and clinically useful therapeutic agents should be effective in reducing cell proliferation. Therefore, these results show the potential utility of BA-1037 in the treatment of malignant melanoma. This potential is especially interesting given the ability of Rho kinase inhibitors to reduce cell migration, and potentially metastasis.

FIG. 12 shows the ability of BA-1037 to reduce proliferation of human endometrial adenocarcinoma cancer cells, HEC-1B. While these cells proliferate more slowly than melanoma cells, BA-1037 was able to completely block proliferation at the highest concentrations tested.

In summary, the experiments that show the ability of BA-1037 to reduce cell proliferation highlight the potential use of these new Rho kinase inhibitors for the treatment of various types of cancerous lesions and malignant tumours.

We claim:

1. A compound of formula (I) 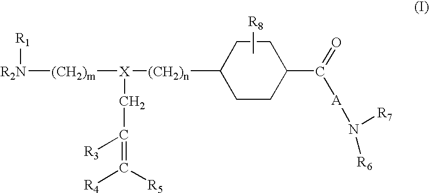
and pharmaceutically acceptable salts thereof,
wherein
X is CH or N, m is 0, 1, 2 or 3 and n is 0, 1, 2 or 3
wherein
R₁ is selected from the group consisting of H, alkyl, cycloalkyl, cycloalkylalkyl, aryl, aralkyl and heteroaryl, a ring group optionally having a substituent on the ring thereof, and
R₂ is selected from the group consisting of H, alkyl, cycloalkyl, cycloalkylalkyl, aryl, aralkyl and heteroaryl, a ring group optionally having a substituent on the ring thereof, or R₁ and R₂ together with the adjacent nitrogen atom form a heterocyclic group optionally having in the ring an oxygen atom, a sulfur atom or an additional nitrogen atom, the heterocyclic group optionally having a substituent on the ring thereof,
and wherein
R₃ is selected from the group consisting of H, halo, alkyl, cycloalkyl, cycloalkylalkyl, aryl, aralkyl and heteroaryl, a ring group optionally having a substituent on the ring thereof,
R₄ is selected from the group consisting of H, halo, alkyl, cycloalkyl, cycloalkylalkyl, aryl, aralkyl and heteroaryl, a ring group optionally having a substituent on the ring thereof,
R₅ is selected from the group consisting of H, halo, alkyl, cycloalkyl, cycloalkylalkyl, aryl, aralkyl and heteroaryl, a ring group optionally having a substituent on the ring thereof,
R₆ is selected from the group consisting of H, alkyl, aryl, and aralkyl,
R₇ is selected from the group consisting of aryl, aralkyl, and heterocyclic groups containing at least one nitrogen atom in the ring structure thereof, a ring group optionally having a substituent on the ring thereof,
R₈ is selected from the group consisting of H, alkyl, halo cycloalkyl, cycloalkylalkyl, aryl, arylalky, a ring group optionally having a substituent on the ring thereof, and
A is a single bond or is an unsubstituted straight chain alkylene group or a straight chain alkylene group substituted by alkyl of 1 to 4 carbon atoms.

2. A compound of formula (I) and pharmaceutically acceptable salts thereof as defined in claim 1, wherein R₇ is selected from the group consisting of
a group of formula (i) 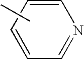
a group of formula (ii) 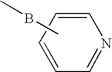
a group of formula (iii) 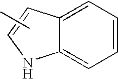
a group of formula (iv) 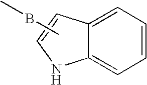
a group of formula (v) 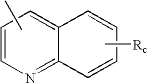
a group of formula (vi) 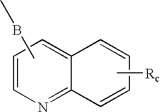
a group of formula (vii) 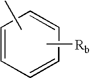
a group of formula (viii) 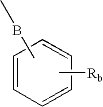
a group of formula (ix) 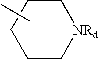
a group of formula (x) 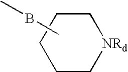
a group of formula (xi) 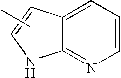
a group of formula (xii) 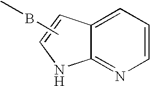
a group of formula (xiii) 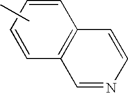
a group of formula (xiv) 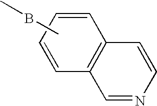
a group of formula (xv) 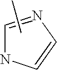
a group of formula (xvi) 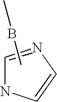
a group of formula (xvii) 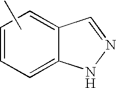
a group of formula (xviii) 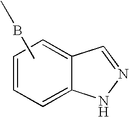
a group of formula (xix) 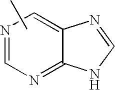
and
a group of formula (xx) 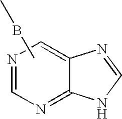
wherein B is an unsubstituted straight chain alkylene group or a straight chain alkylene group substituted by alkyl of 1 to 4 carbon atoms, and R_{b} is selected from the group consisting of H, alkyl, amino, alkylamino, dialkylamino, R_{c} is selected from the group consisting of H, alkyl and R_{d} is selected from the group consisting of H, alkyl, aralkyl.

3. A compound of formula (I) and pharmaceutically acceptable salts thereof as defined in claim 2 wherein
the group of formula (i) is 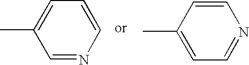
the group of formula (ii) is 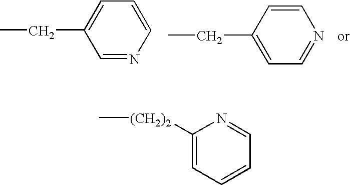
the group of formula (iii) is 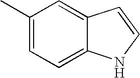
the group of formula (iv) is 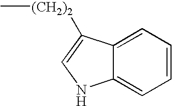
a group of formula (v) is 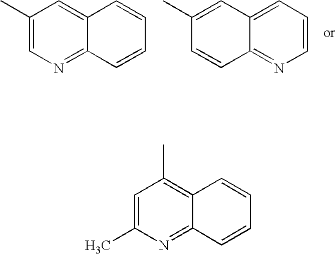
the group of formula (viii) is 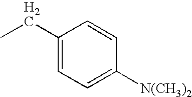
the group of formula (ix) is 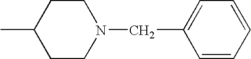
the group of formula (xi) is 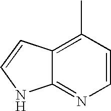
the group of formula (xiii) is 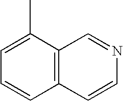
the group of formula (xv) is 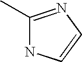
the group of formula (xvii) is 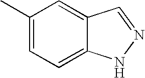
the group of formula (xix) is

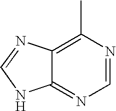

4. A compound of formula (I) and pharmaceutically acceptable salts thereof as defined in claim 1 wherein X is CH.

5. A compound of formula (I) and pharmaceutically acceptable salts thereof as defined in claim 4 wherein R₂, R₃, R₄, R₅, R₆, and R₈ are each H.

6. A compound of formula (I) and pharmaceutically acceptable salts thereof as defined in claim 5 wherein R₁ is selected from the group consisting of H, C₁ to C₆ alkyl, and benzyl.

7. A compound of formula (I) and pharmaceutically acceptable salts thereof as defined in claim 2 wherein X is CH.

8. A compound of formula (I) and pharmaceutically acceptable salts thereof as defined in claim 7 wherein R₂, R₃, R₄, R₅, R₆, and R₈ are each H.

9. A compound of formula (I) and pharmaceutically acceptable salts thereof as defined in claim 8 wherein R₁ is selected from the group consisting of H, C₁ to C₆ alkyl, and benzyl.

10. A compound of formula (I) and pharmaceutically acceptable salts thereof as defined in claim 3 wherein X is CH.

11. A compound of formula (I) and pharmaceutically acceptable salts thereof as defined in claim 10 wherein R₂, R₃, R₄, R₅, R₆, and R₈ are each H.

12. A compound of formula (Ib) 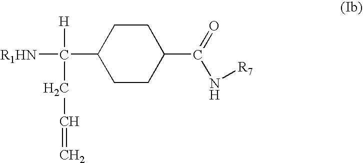
and pharmaceutically acceptable salts thereof
wherein
R₁ is selected from the group consisting of H, C₁ to C₆ alkyl, and benzyl, and
wherein
R₇ is selected from the group consisting of

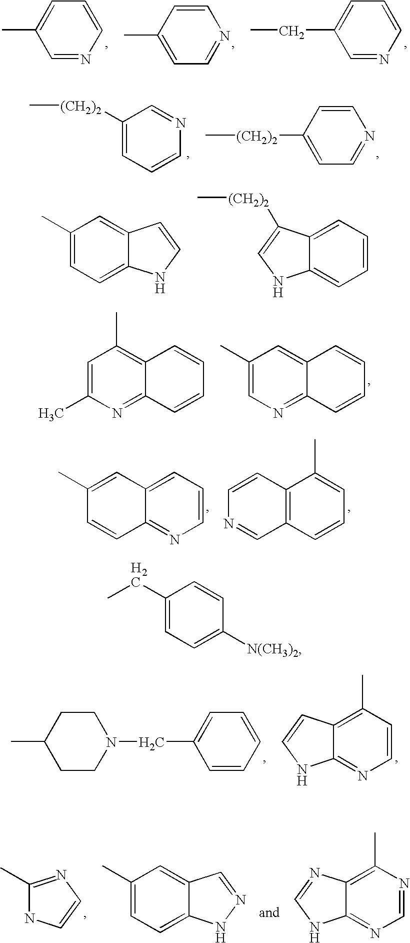

13. A compound of formula (IIa) 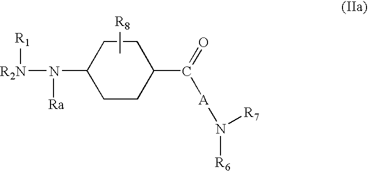
and pharmaceutically acceptable salts thereof
wherein
R₁ is selected from the group consisting of H, alkyl, cycloalkyl, cycloalkylalkyl, aralkyl and heteroaryl, a ring group optionally having a substituent on the ring thereof, and
R₂ is selected from the group consisting of H, alkyl, cycloalkyl, cycloalkylalkyl, aryl, aralkyl and heteroaryl, a ring group optionally having a substituent on the ring thereof, or
R₁ and R₂ together with the adjacent nitrogen atom form a heterocyclic group optionally having in the ring an oxygen atom, a sulfur atom or an additional nitrogen atom, the heterocyclic group optionally having a substituent on the ring thereof,
wherein
Ra is selected from the group consisting of H, alkyl, cycloalkyl, cycloalkylalkyl, aryl, aralkyl, arylalkylene, aryloxyaryl, heteroaryl, a ring group optionally having a substituent on the ring thereof and an alkylene group of formula 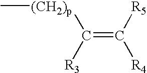
wherein
p is 0, 1, 2 or 3,
R₃ is selected from the group consisting of H, halo, alkyl, cycloalkyl, cycloalkylalkyl, aryl, aralkyl and heteroaryl, a ring group optionally having a substituent on the ring thereof,
R₄ is selected from the group consisting of H, halo, alkyl, cycloalkyl, cycloalkylalkyl, aryl, aralkyl and heteroaryl, a ring group optionally having a substituent on the ring thereof,
R₅ is selected from the group consisting of H, halo, alkyl, cycloalkyl, cycloalkylalkyl, aryl, aralkyl and heteroaryl, a ring group optionally having a substituent on the ring thereof,
and wherein
R₆ is selected from the group consisting of H, alkyl, aryl, and aralkyl,
R₇ is selected from the group consisting of aryl, aralkyl, and heterocyclic groups containing at least one nitrogen atom in the ring structure thereof, a ring group optionally having a substituent on the ring thereof,
R₈ is selected from the group consisting of H, alkyl, halo, cycloalkyl, cycloalkylalkyl, aryl, arylalkyl, a ring group optionally having a substituent on the ring thereof, and
A is a single bond or is an unsubstituted straight chain alkylene group or a straight chain alkylene group substituted by alkyl of 1 to 4 carbon atoms.

14. A compound of formula (IIa) and pharmaceutically acceptable salts thereof as defined in claim 13 wherein R₇ is selected from the group consisting of
a group of formula (i) 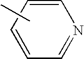
a group of formula (ii) 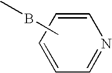
a group of formula (iii) 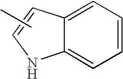
a group of formula (iv) 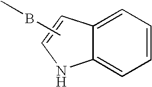
a group of formula (v) 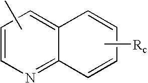
a group of formula (vi) 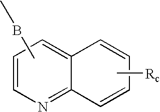
a group of formula (vii) 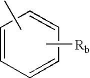
a group of formula (viii) 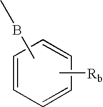
a group of formula (ix) 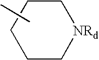
a group of formula (x) 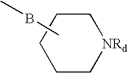
a group of formula (xi) 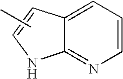
a group of formula (xii) 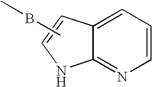
a group of formula (xiii) 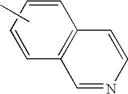
a group of formula (xiv) 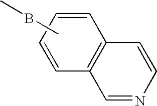
a group of formula (xv) 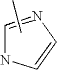
a group of formula (xvi) 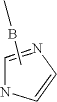
a group of formula (xvii) 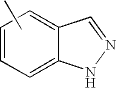
a group of formula (xviii) 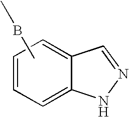
a group of formula (xix) 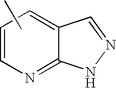
and
a group of formula (xx) 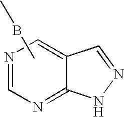
wherein B is an unsubstituted straight chain alkylene group or a straight chain alkylene group substituted by alkyl of 1 to 4 carbon atoms, and R_{b} is selected from the group consisting of H, alkyl, amino, alkylamino, dialkylamino, R_{c} is selected from the group consisting of H, alkyl and R_{d} is selected from the group consisting of H, alkyl, aralkyl.

15. A compound of formula (IIa) and pharmaceutically acceptable salts thereof as defmed in claim 13 wherein R₇ is selected from the group consisting

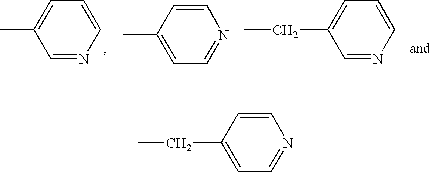

16. A compound of formula (II) and pharmaceutically acceptable salts thereof as defined in claim 13 wherein R₂, R₆, and R₈ are each H.

17. A compound of formula (IIa) and pharmaceutically acceptable salts thereof as defined in claim 16 wherein R₁ is selected from the group consisting of H, C₁ to C₆ alkyl, and benzyl.

18. A compound of formula (IIa) and pharmaceutically acceptable salts thereof as defined in claim 14 wherein R₂, R₆, and R₈ are each H.

19. A compound of formula (IIa) and pharmaceutically acceptable salts thereof as defined in claim 18 wherein R₁ is selected from the group consisting of H, C₁ to C₆ alkyl, and benzyl.

20. A compound of formula (IIa) and pharmaceutically acceptable salts thereof as defined in claim 19 wherein Ra is selected from the group consisting of H, C₁ to C10 alkyl, cyclohexyl(C₁ to C₃)alkyl, phenyl(C₁ to C₃)alkyl, diphenyl(C₁ to C₃)alkyl, phenyl(C₂ to C₃)alkylene, benzyloxybenzyl and allyl.

21. A compound of formula (IIa) and pharmaceutically acceptable salts thereof as defined in claim 15 wherein R₂, R₆, and R₈ are each H.

22. A compound of formula (IIb) 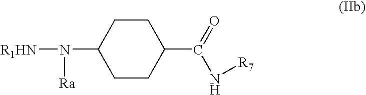
and pharmaceutically acceptable salts thereof
wherein
R₁ is selected from the group consisting of H, C₁ to C₆ alkyl, and benzyl,
wherein
R₇ is selected from the group consisting of 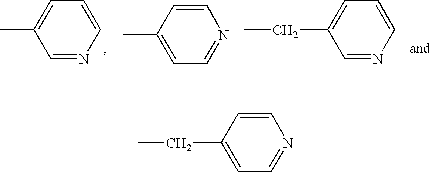
and
wherein Ra is selected from the group consisting of H, C₁ to C₁₀ alkyl, cyclohexyl(C₁ to C₃)alkyl, phenyl(C₁ to C₃)alkyl, diphenyl(C₁ to C₃)alkyl, phenyl(C₂ to C₃)alkylene, benzyloxybenzyl and allyl.

23. A compound of formula (I) and pharmaceutically acceptable salts thereof as defined in claim 1 selected from the group consisting of
4-(But-3′-en-1′-amino)-N-(4″-pyridyl)cyclohexane Carboxamide,
4-(But-3′-en-1′-amino)-N-[2″-(3′″-indolyl)ethyl]cyclohexane Carboxamide,
4-(But-3′-en-1′-amino)-N-[(3″-pyridyl)methyl]cyclohexane Carboxamide,
4-(But-3′-en-1′-amino)-N-[2″-(2′″-pyridyl)ethyl]cyclohexane Carboxamide,
4-(But-3′-en-1′-amino)-N-[4″-(N″-benzyl)piperidyl]-cyclohexane Carboxamide,
4-(But-3′-en-1′-amino)-N-(3″-pyridyl)cyclohexane Carboxamide,
4-(But-3′-en-1′-amino)-N-(3″-quinolyl)cyclohexane Carboxamide,
4-(But-3′-en-1′-amino)-N-(5″-isoquinolyl)cyclohexane Carboxamide,
4-(But-3′-en-1′-amino)-N-(6″-quinolyl)cyclohexane Carboxamide,
4-(But-3′-en-1′-amino)-N-[4″-(dimethylamino)-benzyl]cyclohexane Carboxamide,
4-(But-3′-en-1′-amino)-N-(4″-quinaldyl)cyclohexane Carboxamide,
4-(But-3′-en-1′-amino)-N-(5″-indolyl)cyclohexane Carboxamide,
4-(But-3′-en-1′-amino)-N-[(4″-pyridyl)methyl]cyclohexane Carboxamide,
4-(But-3′-en-1′-amino)-N-(4″-pyridyl)cyclohexane Carboxamide,
4-[(N′-methyl)-but-3′-en-1′-amino]-N-(4″-pyridyl)cyclohexane Carboxamide,
4-[(N′-benzyl)-but-3′-en-1′-amino]-N-(4″-pyridyl)cyclohexane Carboxamide,
4-(But-3′-en-1′-amino)-N-(6″-puryl)cyclohexane Carboxamide,
and pharmaceutically acceptable salts thereof.

24. A compound of formula (I) and pharmaceutically acceptable salts thereof as defined in claim 1 selected from the group consisting of
(R,S)-trans-4-(But-3′-en-1′-amino)-N-(4″-pyridyl)cyclohexane Carboxamide,
(R,S)-trans-4-(But-3′-en-1′-amino)-N-[2″-(3′″-indolyl)ethyl]cyclohexane Carboxamide,
(R,S)-trans-4-(But-3′-en-1′-amino)-N-[(3″-pyridyl)methyl]cyclohexane Carboxamide,
(R,S)-trans-4-(But-3′-en-1′-amino)-N-[2″-(2′″-pyridyl)ethyl]cyclohexane Carboxamide,
(R,S)-trans-4-(But-3′-en-1′-amino)-N-[4″-(N″-benzyl)piperidyl]-cyclohexane Carboxamide,
(R,S)-trans-4-(But-3′-en-1′-amino)-N-(3″-pyridyl)cyclohexane Carboxamide,
(R,S)-trans-4-(But-3′-en-1′-amino)-N-(3″-quinolyl)cyclohexane Carboxamide,
(R,S)-trans-4-(But-3′-en-1′-amino)-N-(5″-isoquinolyl)cyclohexane Carboxamide,
(R,S)-trans-4-(But-3′-en-1′-amino)-N-(6″-quinolyl)cyclohexane Carboxamide,
(R,S)-trans-4-(But-3′-en-1′-amino)-N-[4″-(dimethylamino)-benzyl]cyclohexane Carboxamide,
(R,S)-trans-4-(But-3′-en-1′-amino)-N-(4″-quinaldyl)cyclohexane Carboxamide,
(R,S)-trans-4-(But-3′-en-1′-amino)-N-(5″-indolyl)cyclohexane Carboxamide,
(R,S)-trans-4-(But-3′-en-1′-amino)-N-[(4″-pyridyl)methyl]cyclohexane Carboxamide,
(R)-trans-4-(But-3′-en-1′-amino)-N-(4″-pyridyl)cyclohexane Carboxamide,
(S)-trans-4-(But-3′-en-1′-amino)-N-(4″-pyridyl)cyclohexane Carboxamide,
(R,S)-trans-4-[(N′-methyl)-but-3′-en-1′-amino]-N-(4″-pyridyl)cyclohexane Carboxamide,
(R,S)-trans-4-[(N′-benzyl)-but-3′-en-1′-amino]-N-(4″-pyridyl)cyclohexane Carboxamide,
(R,S)-trans-4-(But-3′-en-1′-amino)-N-(6″-puryl)cyclohexane Carboxamide,
and pharmaceutically acceptable salts thereof.

25. A salt of a compound of formula (I) as defined in claim 1 selected from the group consisting of
(R,S)-trans-4-(But-3′-en-1′-amino)-N-(4″-pyridyl)cyclohexane Carboxamide Dihydrochloride,
(R,S)-trans-4-(But-3′-en-1′-amino)-N-[2″-(3′″-indolyl)ethyl]cyclohexane Carboxamide Dihydrochloride,
(R,S)-trans-4-(But-3′-en-1′-amino)-N-[(3″-pyridyl)methyl]cyclohexane Carboxamide Dihydrochloride,
(R,S)-trans-4-(But-3′-en-1′-amino)-N-[2″-(2′″-pyridyl)ethyl]cyclohexane Carboxamide Dihydrochloride,
(R,S)-trans-4-(But-3′-en-1′-amino)-N-[4″-(N″-benzyl)piperidyl]-cyclohexane Carboxamide Dihydrochloride,
(R,S)-trans-4-(But-3′-en-1′-amino)-N-(3″-pyridyl)cyclohexane Carboxamide Dihydrochloride,
(R,S)-trans-4-(But-3′-en-1′-amino)-N-(3″-quinolyl)cyclohexane Carboxamide Dihydrochloride,
(R,S)-trans-4-(But-3′-en-1′-amino)-N-(5″-isoquinolyl)cyclohexane Carboxamide Dihydrochloride,
(R,S)-trans-4-(But-3′-en-1′-amino)-N-(6″-quinolyl)cyclohexane Carboxamide Dihydrochloride,
(R,S)-trans-4-(But-3′-en-1′-amino)-N-[4″-(dimethylamino)-benzyl]cyclohexane Carboxamide Dihydrochloride,
(R,S)-trans-4-(But-3′-en-1′-amino)-N-(4″-quinaldyl)cyclohexane Carboxamide Dihydrochloride,
(R,S)-trans-4-(But-3′-en-1′-amino)-N-(5″-indolyl)cyclohexane Carboxamide Dihydrochloride,
(R,S)-trans-4-(But-3′-en-1′-amino)-N-[(4″-pyridyl)methyl]cyclohexane Carboxamide Dihydrochloride,
(R)-trans-4-(But-3′-en-1′-amino)-N-(4″-pyridyl)cyclohexane Carboxamide Dihydrochloride,
(S)-trans-4-(But-3′-en-1′-amino)-N-(4″-pyridyl)cyclohexane Carboxamide Dihydrochloride,
(R,S)-trans-4-[(N′-methyl)-but-3′-en-1′-amino]-N-(4″-pyridyl)cyclohexane Carboxamide Dihydrochloride,
(R,S)-trans-4-[(N′-benzyl)-but-3′-en-1′-amino]-N-(4″-pyridyl)cyclohexane Carboxamide Dihydrochloride,
and
(R,S)-trans-4-(But-3′-en-1′-amino)-N-(6″-puryl)cyclohexane Carboxamide Dihydrochloride.

26. A compound of formula (IIa) and pharmaceutically acceptable salts thereof as defined in claim 13 selected from the group consisting of
4-[N′-(Methyl)hydrazino]-N-(4″-pyridyl)cyclohexane Carboxamide,
4-[N′-(Propyl)hydrazino]-N-(4″-pyridyl)cyclohexane Carboxamide,
4-{N′-[3′-(Methyl)butyl]hydrazino}-N-(4″-pyridyl)cyclohexane Carboxamide,
4-{N′-[1′-(Methyl)ethyl]hydrazino}-N-(4″-pyridyl)cyclohexane Carboxamide,
4-[N′-(Benzyl)hydrazino]-N-(4″-pyridyl)cyclohexane Carboxamide,
4-{N′-[2′-(Phenyl)ethyl]hydrazino}-N-(4′″-pyridyl)cyclohexane Carboxamide,
4-{N′-[2′,2′-(Diphenyl)ethyl]hydrazino}-N-(4′″-pyridyl)cyclohexane Carboxamide,
4-{N′-[4′-(Benzyloxy)benzyl]hydrazino}-N-(4′″-pyridyl)cyclohexane Carboxamide,
4-{N′-[(Cyclohexyl)methyl]hydrazino}-N-(4′″-pyridyl)cyclohexane Carboxamide,
4-[N′-(Octyl)hydrazino]-N-(4′″-pyridyl)cyclohexane Carboxamide,
4-{N′-[3′-(Phenyl)prop-2′-enyl]hydrazino}-N-(4′″-pyridyl)cyclohexane Carboxamide,
and pharmaceutically acceptable salts thereof.

27. A compound of formula (IIa) and pharmaceutically acceptable salts thereof as defined in claim 13 selected from the group consisting of
cis-4-[N′-(Methyl)hydrazino]-N-(4″-pyridyl)cyclohexane Carboxamide,
trans-4-[N′-(Methyl)hydrazino]-N-(4″-pyridyl)cyclohexane Carboxamide,
trans-4-[N′-(Propyl)hydrazino]-N-(4″-pyridyl)cyclohexane Carboxamide,
trans-4-{N′-[3′-(Methyl)butyl]hydrazino}-N-(4″-pyridyl)cyclohexane Carboxamide,
trans-4-{N′-[1′-(Methyl)ethyl]hydrazino}-N-(4″-pyridyl)cyclohexane Carboxamide Dihydrochloride,
trans-4-[N′-(Benzyl)hydrazino]-N-(4″-pyridyl)cyclohexane Carboxamide,
cis-4-[N′-(Propyl)hydrazino]-N-(4″-pyridyl)cyclohexane Carboxamide,
cis-4-{N′-[3′-(Methyl)butyl]hydrazino}-N-(4″-pyridyl)cyclohexane Carboxamide,
cis-4-{N′-[1′-(Methyl)ethyl]hydrazino}-N-(4″-pyridyl)cyclohexane Carboxamide,
cis-4-[N′-(Benzyl)hydrazino]-N-(4″-pyridyl)cyclohexane Carboxamide,
trans-4-{N′-[2′-(Phenyl)ethyl]hydrazino}-N-(4′″-pyridyl)cyclohexane Carboxamide,
trans-4-{N′-[2′,2′-(Diphenyl)ethyl]hydrazino}-N-(4′″-pyridyl)cyclohexane Carboxamide,
trans-4-{N′-[4′-(Benzyloxy)benzyl]hydrazino}-N-(4′″-pyridyl)cyclohexane Carboxamide,
trans-4-{N′-[(Cyclohexyl)methyl]hydrazino}-N-(4′″-pyridyl)cyclohexane Carboxamide,
trans-4-[N′-(Octyl)hydrazino]-N-(4′″-pyridyl)cyclohexane Carboxamide,
1,4-trans-2′,3′-trans-4-{N′-[3′-(Phenyl)prop-2′-enyl]hydrazino}-N-(4′″-pyridyl)cyclohexane Carboxamide,
and pharmaceutically acceptable salts thereof.

28. A salt of a compound of formula (IIa) as defined in claim 13 selected from the group consisting of
cis-4-[N′-(Methyl)hydrazino]-N-(4″-pyridyl)cyclohexane Carboxamide Dihydrochloride,
trans-4-[N′-(Methyl)hydrazino]-N-(4″-pyridyl)cyclohexane Carboxamide Dihydrochloride,
trans-4-[N′-(Propyl)hydrazino]-N-(4″-pyridyl)cyclohexane Carboxamide Dihydrochloride,
trans-4-{N′-[3′-(Methyl)butyl]hydrazino}-N-(4″-pyridyl)cyclohexane Carboxamide Dihydrochloride,
trans-4-{N′-[1′-(Methyl)ethyl]hydrazino}-N-(4″-pyridyl)cyclohexane Carboxamide Dihydrochloride,
trans-4-[N′-(Benzyl)hydrazino]-N-(4″-pyridyl)cyclohexane Carboxamide Dihydrochloride,
cis-4-[N′-(Propyl)hydrazino]-N-(4″-pyridyl)cyclohexane Carboxamide Dihydrochloride,
cis-4-{N′-[3′-(Methyl)butyl]hydrazino}-N-(4″-pyridyl)cyclohexane Carboxamide Dihydrochloride,
cis-4-{N′-[1′-(Methyl)ethyl]hydrazino}-N-(4″-pyridyl)cyclohexane Carboxamide Dihydrochloride,
cis-4-[N′-(Benzyl)hydrazino]-N-(4″-pyridyl)cyclohexane Carboxamide Dihydrochloride,
trans-4-{N′-[2′-(Phenyl)ethyl]hydrazino}-N-(4′″-pyridyl)cyclohexane Carboxamide Dihydrochloride,
trans-4-{N′-[2′,2′-(Diphenyl)ethyl]hydrazino}-N-(4′″-pyridyl)cyclohexane Carboxamide Dihydrochloride,
trans-4-{N′-[4′-(Benzyloxy)benzyl]hydrazino}-N-(4′″-pyridyl)cyclohexane Carboxamide Dihydrochloride,
trans-4-{N′-[(Cyclohexyl)methyl]hydrazino}-N-(4′″-pyridyl)cyclohexane Carboxamide Dihydrochloride,
trans-4-[N′-(Octyl)hydrazino]-N-(4′″-pyridyl)cyclohexane Carboxamide Dihydrochloride,
and
1,4-trans-2′,3′-trans-4-{N′-[3′-(Phenyl)prop-2′-enyl]hydrazino}-N-(4′″-pyridyl)cyclohexane Carboxamide Dihydrochloride.

29. A compound of formula (I) and pharmaceutically acceptable salts thereof as defmed in claim 1 selected from the group consisting of
4-(But-3′-en-1′-amino)-N-(4″-pyridyl)cyclohexane Carboxamide
and pharmaceutically acceptable salts thereof.

30. A compound of formula (I) and pharmaceutically acceptable salts thereof as defined in claim 1 selected from the group consisting of
(R)-trans-4-(But-3′-en-1′-amino)-N-(4″-pyridyl)cyclohexane Carboxamide
and pharmaceutically acceptable salts thereof.

31. A salt of a compound of formula (I) as defined in claim 1 said salt being
(R)-trans-4-(But-3′-en-1′-amino)-N-(4″-pyridyl)cyclohexane Carboxamide Dihydrochloride.

32. A compound of formula (IIa) and pharmaceutically acceptable salts thereof as defined in claim 13 selected from the group consisting of
4-[N′-(Octyl)hydrazino]-N-(4′″-pyridyl)cyclohexane Carboxamide
and pharmaceutically acceptable salts thereof.

33. A compound of formula (IIa) and pharmaceutically acceptable salts thereof as defined in claim 13 selected from the group consisting of
trans-4-[N′-(Octyl)hydrazino]-N-(4′″-pyridyl)cyclohexane Carboxamide
and pharmaceutically acceptable salts thereof.

34. A salt of a compound of formula (IIa) as defined in claim 13, said salt being
trans-4-[N′-(Octyl)hydrazino]-N-(4′″-pyridyl)cyclohexane Carboxamide Dihydrochloride.

35. A pharmaceutical composition comprising a pharmaceutically acceptable carrier and a member of the group consisting of a compound of formula (I) and pharmaceutically acceptable salt thereof as defined in claim 1.

36. A pharmaceutical composition comprising a pharmaceutically acceptable carrier and a member of the group consisting of a compound of formula (Ib) and pharmaceutically acceptable salts thereof as defined in claim 12.

37. A pharmaceutical composition comprising a pharmaceutically acceptable carrier and a member of the group consisting of a compound of formula (IIa) and pharmaceutically acceptable salts thereof as defined in claim 13.

38. A pharmaceutical composition comprising a pharmaceutically acceptable carrier and a member of the group consisting of a compound of formula (IIa) and pharmaceutically acceptable salts thereof as defined in claim 22.

39. A pharmaceutical composition comprising a pharmaceutically acceptable carrier and a member of the group consisting of a compound of formula (I) and pharmaceutically acceptable salts thereof as defined in claim 23.

40. A pharmaceutical composition comprising a pharmaceutically acceptable carrier and a member of the group consisting of a compound of formula (I) and pharmaceutically acceptable salts thereof as defined in claim 24.

41. A pharmaceutical composition comprising a pharmaceutically acceptable carrier and a member of the group consisting of a pharmaceutically acceptable salt of a compound of formula (I) as defmed in claim 25.

42. A pharmaceutical composition comprising a pharmaceutically acceptable carrier and a member of the group consisting of a compound of formula (IIa) and pharmaceutically acceptable salts thereof as defined in claim 26.

43. A pharmaceutical composition comprising a pharmaceutically acceptable carrier and a member of the group consisting of a compound of formula (IIa) and pharmaceutically acceptable salts thereof as defined in claim 27.

44. A pharmaceutical composition comprising a pharmaceutically acceptable carrier and a member of the group consisting of a pharmaceutically acceptable salt of a compound of formula (IIa) as defined in claim 28.

45. A pharmaceutical composition comprising a pharmaceutically acceptable carrier and a member of the group consisting of a compound of formula (I) and pharmaceutically acceptable salts thereof as defined in claim 29.

46. A pharmaceutical composition comprising a pharmaceutically acceptable carrier and a member of the group consisting of a compound of formula (I) and pharmaceutically acceptable salts thereof as defined in claim 30.

47. A pharmaceutical composition comprising a pharmaceutically acceptable carrier and a pharmaceutically acceptable salt of a compound of formula (I) as defined in claim 31.

48. A pharmaceutical composition comprising a pharmaceutically acceptable carrier and a member of the group consisting of a compound of formula (IIa) and pharmaceutically acceptable salts thereof as defined in claim 32.

49. A pharmaceutical composition comprising a pharmaceutically acceptable carrier and a member of the group consisting of a compound of formula (IIa) and pharmaceutically acceptable salts thereof as defined in claim 33.

51. A pharmaceutical composition comprising a pharmaceutically acceptable carrier and a pharmaceutically acceptable salt of a compound of formula (IIa) as defined in claim 34.

52. A compound of formula (II) 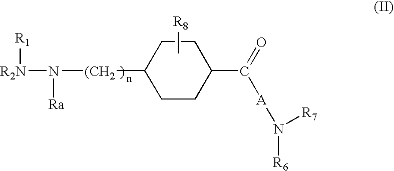
and pharmaceutically acceptable salts thereof
wherein
R₁ is selected from the group consisting of H, alkyl, cycloalkyl, cycloalkylalkyl, aralkyl and heteroaryl, a ring group optionally having a substituent on the ring thereof, and
R₂ is selected from the group consisting of H, alkyl, cycloalkyl, cycloalkylalkyl, aryl, aralkyl and heteroaryl, a ring group optionally having a substituent on the ring thereof, or
R₁ and R₂ together with the adjacent nitrogen atom form a heterocyclic group optionally having in the ring an oxygen atom, a sulfur atom or an additional nitrogen atom, the heterocyclic group optionally having a substituent on the ring thereof,
wherein
Ra is selected from the group consisting of H, alkyl, cycloalkyl, cycloalkylalkyl, aryl, aralkyl, arylalkylene, aryloxyaryl, heteroaryl, a ring group optionally having a substituent on the ring thereof, and an alkylene group of formula 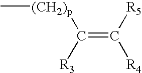
wherein
p is 0, 1, 2 or 3,
R₃ is selected from the group consisting of H, halo, alkyl, cycloalkyl, cycloalkylalkyl, aryl, aralkyl and heteroaryl, a ring group optionally having a substituent on the ring thereof, R₄ is selected from the group consisting of H, halo, alkyl, cycloalkyl, cycloalkylalkyl, aryl, aralkyl and heteroaryl, a ring group optionally having a substituent on the ring thereof,
R₅ is selected from the group consisting of H, halo, alkyl, cycloalkyl, cycloalkylalkyl, aryl, aralkyl and heteroaryl, a ring group optionally having a substituent on the ring thereof,
and wherein
R₆ is selected from the group consisting of H, alkyl, aryl, and aralkyl,
R₇ is selected from the group consisting of aryl, aralkyl, and heterocyclic groups containing at least one nitrogen atom in the ring structure thereof, a ring group optionally having a substituent on the ring thereof,
R₈ is selected from the group consisting of H, alkyl, halo, cycloalkyl, cycloalkylalkyl, aryl, arylalkyl, a ring group optionally having a substituent on the ring thereof, and
A is a single bond or is an unsubstituted straight chain alkylene group or a straight chain alkylene group substituted by alkyl of 1 to 4 carbon atoms.

53. A compound of formula (II) and pharmaceutically acceptable salts thereof as defined in claim 52 wherein R₇ is selected from the group consisting of
a group of formula (i) 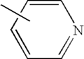
a group of formula (ii) 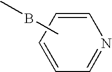
a group of formula (iii) 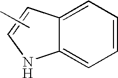
a group of formula (iv) 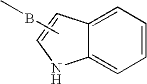
a group of formula (v) 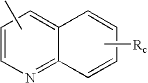
a group of formula (vi) 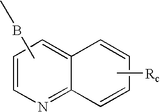
a group of formula (vii) 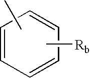
a group of formula (viii) 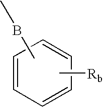
a group of formula (ix) 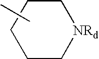
a group of formula (x) 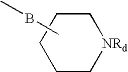
a group of formula (xi) 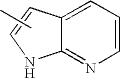
a group of formula (xii) 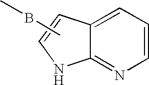
a group of formula (xiii) 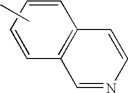
a group of formula (xiv) 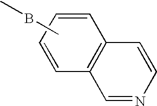
a group of formula (xv) 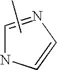
a group of formula (xvi) 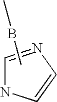
a group of formula (xvii) 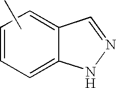
a group of formula (xviii) 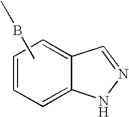
a group of formula (xix) 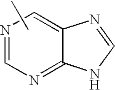
and
a group of formula (xx) 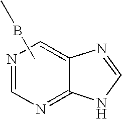
wherein B is an unsubstituted straight chain alkylene group or a straight chain alkylene group substituted by alkyl of 1 to 4 carbon atoms, and R_{b} is selected from the group consisting of H, alkyl, amino, alkylamino, dialkylamino, R_{c} is selected from the group consisting of H, alkyl and R_{d} is selected from the group consisting of H, alkyl, aralkyl.

54. A pharmaceutical composition comprising a pharmaceutically acceptable carrier and a member of the group consisting of a compound of formula (II) and pharmaceutically acceptable salts thereof as defined in claim 52.

55. A compound of formula (I) and pharmaceutically acceptable salts thereof as defined in claim 1 wherein X is CH and A is a single bond.

56. A compound of formula (I) and pharmaceutically acceptable salts thereof as defined in claim 55 wherein R₂, R₃, R₄, R₅, R₆, and R₈ are each H.

57. A compound of formula (I) and pharmaceutically acceptable salts thereof as defined in claim 56 wherein R₁ is selected from the group consisting of H, C₁ to C₆ alkyl, and benzyl.

58. A compound of formula (I) and pharmaceutically acceptable salts thereof as defined in claim 2 wherein X is CH and A is a single bond.

59. A compound of formula (I) and pharmaceutically acceptable salts thereof as defined in claim 58 wherein R₂, R₃, R₄, R₅, R₆, and R₈ are each H.

60. A compound of formula (I) and pharmaceutically acceptable salts thereof as defined in claim 59 wherein R₁ is selected from the group consisting of H, C₁ to C₆ alkyl, and benzyl.

61. A compound of formula (I) and pharmaceutically acceptable salts thereof as defined in claim 3 wherein X is CH and A is a single bond.

62. A compound of formula (I) and pharmaceutically acceptable salts thereof as defined in claim 61 wherein R₂, R₃, R₄, R₅, R₆, and R₈ are each H.

63. A pharmaceutical composition comprising a pharmaceutically acceptable carrier and a member of the group,consisting of a compound of formula (I) and pharmaceutically acceptable salt thereof as defined in claim 55.

64. A pharmaceutical composition comprising a pharmaceutically acceptable carrier and a member of the group consisting of a compound of formula (I) and pharmaceutically acceptable salt thereof as defined in claim 58.

65. A pharmaceutical composition comprising a pharmaceutically acceptable carrier and a member of the group consisting of a compound of formula (I) and pharmaceutically acceptable salt thereof as defined in claim 61.
